(19) United States
(12) Patent Application Publication
(10) Pub. No.: US 2025/0214956 A1
(43) Pub. Date: Jul. 3, 2025

(54) ORGANIC ELECTRONIC DEVICE AND DISPLAY APPARATUS USING COMPOSITION FOR ORGANIC ELECTRONIC DEVICE

(71) Applicant: DUK SAN NEOLUX CO., LTD., Cheonan-si (KR)

(72) Inventors: Mun Jae LEE, Cheonan-si (KR); Jung Cheol PARK, Suwon-si (KR); Soung Yun MUN, Cheonan-si (KR); Jae Taek KWON, Jeonju-si (KR); Bum Sung LEE, Cheonan-si (KR)

(73) Assignee: DUK SAN NEOLUX CO., LTD., Cheonan-si (KR)

(21) Appl. No.: 19/086,848

(22) Filed: Mar. 21, 2025

Related U.S. Application Data
(63) Continuation of application No. 15/521,463, filed on Apr. 24, 2017.

(30) Foreign Application Priority Data
Oct. 24, 2014 (KR) 10-2014-0145033

(51) Int. Cl. C07D 307/77; C07C 211/54; C07D 307/91; C07D 333/50; C07D 333/74; C07D 333/76; C07D 407/14; C07D 409/12; C07D 409/14; C07D 495/04; C09K 11/06; H10K 30/80; H10K 50/11; H10K 50/15; H10K 59/12; H10K 71/00; H10K 71/12; H10K 85/60

(52) U.S. Cl. CPC C07D 307/77; C07C 211/54; C07D 307/91; C07D 333/50; C07D 333/74; C07D 333/76; C07D 407/14; C07D 409/12; C07D 409/14; C07D 495/04; C09K 11/06; H10K 85/631; H10K 85/633; H10K 85/636; C07C 2603/18; C07C 2603/26; C07C 2603/94; H10K 30/865; H10K 50/11; H10K 50/15; H10K 59/12; H10K 71/00; H10K 71/12; H10K 85/615; H10K 85/624; H10K 85/626; H10K 85/657; H10K 85/6574; H10K 85/6576; Y02E 10/549

(57) ABSTRACT

Provided are an organic electric element and a display device using the same as a hole transport layer comprising a composition composed of two or more compounds having similar structures to improve luminous efficiency, stability and life span of an electric element, and an electronic device including the same.

### BACKGROUND

### Technical Field

The present invention relates to organic electric element, display device and electronic device using composition composed of compound for organic electric element, and more specifically, display device and organic electric element comprising the organic layer using two or more different hole transport material in the hole transport layer.

### Background Art

In general, an organic luminescence phenomenon refers to a phenomenon in which electric energy is converted into light energy by means of an organic material. The organic electric element using the organic luminescence phenomenon is, by applying current, self-luminous element using luminescence principle of luminescent material by recombination energy of holes injected from the anode and electron injected from the cathode.

The organic electric element may have a structure in which an anode is formed on a substrate, on which the organic electric element may have a structure formed sequentially a hole injection layer, a hole transport layer, an emitting layer, an electron transport layer, an electron injection layer, and a cathode. Here, the hole injection layer, the hole transport layer, the emitting layer, the electron transport layer, and the electron injection layer are organic thin films made of organic compounds.

Currently, the portable display market is a trend growing in size with large display, which requires larger power consumption than traditional portable displays. Accordingly, power consumption has become an important factor for a portable display having a limited power supply such as battery, and high efficiency, life span and the driving voltage problem are important factors to be solved.

In particular, because life span and driving voltage problem are very relevant to thermal degradation problems of a hole injection material and a hole transport material, a number of methods have been studied in order to overcome this. For example, a method of constituting the hole transport layer in multiple layers (U.S. Pat. No. 5,256,945) and methods of using materials having a high glass transition temperature (U.S. Pat. No. 5,061,569) and so on are proposed.

In addition, when a material having a good hole transport function is used in order to reduce driving voltage, the driving voltage reduction of the element is large, but the charge is excessively injected and the efficiency and life span of the element are lowered, and therefore there have been many attempts to solve these problems.

However, there is a problem that the rise in the progressive driving voltage of the blue organic electric element among the red, green, and blue increases the power consumption and the shortened life span of the organic electric element, and in order to solve such a problem, a technique forming a buffer layer between the anode and the hole transport layer has been proposed (Korean Patent Publication No. 2006-0032099).

### DETAILED DESCRIPTION OF THE INVENTION

### Technical Solution

An object of the present invention is to provide an organic electric element having excellent luminous efficiency by efficiently controlling the injection amount of the charge in the emitting layer to increase the efficiency, and to increase life span by reducing thermal degradation occurring at the interface between the hole injection layer and the hole transport layer and at the interface between the hole transport layer and the emitting layer by mixing two or more hole transport materials having different band gaps in the hole transport layer.

### Technical Solution

The present invention also provides a display device characterized by comprising a first electrode; a second electrode; and an organic material layer; comprising emitting layer disposed between the first electrode and the second electrode and comprising a hole transport layer and light emitting compounds, wherein the hole transport layer is comprised of a mixture of two compounds having different structures among compounds represented by the following Formula 1.

In addition, the present invention relates to the organic electric element and the electronic device thereof using a composition of a compound for an organic electric element represented by the Formula 1, more specially provides an organic electric element and the electronic device including the same using a composition of two or more different hole transport materials in the hole transport layer.

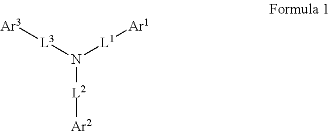

### Effects of the Invention

The organic electric element of the present invention and the display device including the same has a long life span by reducing the thermal degradation occurring at the interface between the hole injection layer and the hole transport layer and at the interface between the hole transport layer and the emitting layer, and has an excellent emitting efficiency by efficiently controlling the injection amount of the charge in the emitting layer.

### BRIEF DESCRIPTION OF THE DRAWINGS

FIG. 1 illustrates an example of an organic electric element according to an embodiment of the present invention.

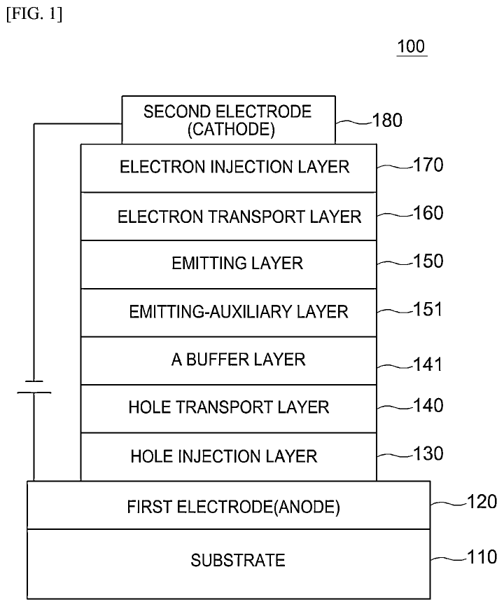

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected “,” coupled” or “connected” between each component.

As used in the specification and the accompanying claims, unless otherwise stated, the following is the meaning of the term as follows.

Unless otherwise stated, the term “halo” or “halogen” as used herein includes fluorine, bromine, chlorine, or iodine.

Unless otherwise stated, the term “alkyl” or “alkyl group” as used herein has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl” as used herein has, but not limited to, double or triple bonds of 2 to 60 carbon atoms, and includes a linear or a branched chain group.

Unless otherwise stated, the term “alkoxyl group”, “alkoxy group” or “alkyloxy group” as used herein means an oxygen radical attached to an alkyl group, but not limited to, and has 1 to 60 carbon atoms.

Unless otherwise stated, the term “aryl group” or “arylene group” as used herein has, but not limited to, 6 to 60 carbon atoms. Herein, the aryl group or the arylene group means a monocyclic or polycyclic aromatic group, and may include the aromatic ring formed in conjunction or reaction with an adjacent substituent. For examples, the aryl group may include a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group” as used herein contains one or more heteroatoms, but not limited to, has 2 to 60 carbon atoms, includes any one of monocyclic or polycyclic rings, and may include heteroaliphadic ring and/or heteroaromatic ring. Also, the heterocyclic group may also be formed in conjunction with an adjacent group.

Unless otherwise stated, the term “heteroatom” as used herein represents at least one of N, O, S, P, or Si.

Also, the term “heterocyclic group” may include a ring comprising SO₂ instead of carbon consisting of cycle. For example, “heterocyclic group” includes compound below.

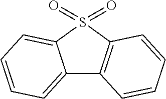 [SMILES: O=S1(=O)c2ccccc2-c2ccccc21]

Unless otherwise stated, the term “aliphatic” as used herein means an aliphatic hydrocarbon having 1 to 60 carbon atoms, and the term “aliphatic ring” as used herein means an aliphatic hydrocarbon ring having 3 to 60 carbon atoms.

Unless otherwise stated, the term “ring” means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds contain, but are not limited to, one or more heteroatoms.

Unless otherwise expressly stated, the term “substituted or unsubstituted” as used herein means that “substitution” is substituted with at least one substituent selected from the group consisting of, but not limited to, deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiophene group, a C₆-C₂₀ arylthiophene group, a C₂-C₂₀ alkenyl group, a C₂-C₂₀ alkynyl group, a C₃-C₂₀ cycloalkyl group, a C₆-C₆₀ aryl group, a C₆-C₂₀ aryl group substituted by deuterium, a C₈-C₂₀ arylalkenyl group, a silane group, a boron group, a germanium group, and a C₂-C₂₀ heterocyclic group.

Unless otherwise expressly stated, the Formula used in the present invention is applied in the same manner as the substituent definition according to the definition of the exponent of the following Formula.

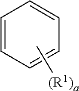

Wherein, when a is an integer of zero, the substituent R¹ is absent, when a is an integer of 1, the sole R¹ is linked to any one of the carbon atoms constituting the benzene ring, when a is an integer of 2 or 3, each substituent R¹s are linked to the benzene ring as follows and may be the same and different. When a is an integer of 4 to 6, the substituent R¹s are linked to carbon of the benzene ring in a similar manner, and the indication of hydrogen bound to the carbon forming the benzene ring is omitted.

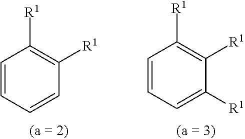

Further, the organic electric element according to the present invention may be any one of an organic light emitting diode (OLED), an organic solar cell, an organic photo conductor (OPC), an organic transistor (organic TFT), and an element for monochromatic or white illumination.

Another embodiment of the present invention may include an electronic device including the display device which includes the described organic electric element of the present invention, and a control unit for controlling the display device. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, a display device and an organic electric element according to an aspect of the present invention will be described.

The present invention provides a display device characterized by comprising a first electrode; a second electrode; and an organic material layer; disposed between the first electrode and the second electrode, and comprising hole transport layer and emitting layer, wherein the hole transport layer is composed of composition of two kinds of arylamine-based compounds having different structures, and the ratio of the compounds of the different structural formulas is selected of any one of 5:5 or 6:4 or 7:3 or 8:2 or 9:1.

According to a specific example of the present invention, the hole transport layer comprises a composition of two compounds having different structures among compounds represented by the following Formula 1.

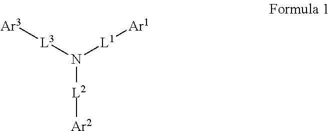

{In the Formula 1, wherein Ar¹, Ar², Ar³ are each independently selected from the group consisting of a C₂-C₆₀ heteroaryl group; a fluorenyl group; L¹, L², L³ are selected from the group consisting of a single bond, a C₆-C₆₀ arylene group, a divalent of C₂-C₆₀ heterocyclic group, a fluorenylene group, a fused ring group of a C₃-C₆₀ aliphatic ring and a C₆-C₆₀ aromatic ring.
(where, aryl group, heteroaryl group, fluorenyl group, arylene group, heterocyclic group and fused ring group may be substituted with one or more substituents selected from deuterium; halogen; a silane group; a siloxane group; a boron group; a germanium group; a cyano group; a nitro group; -L′-N(Rₐ)(R_{b}); a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₆₀ aryl group; a C₆-C₆₀ aryl group substituted with deuterium; a fluorenyl group; a C₂-C₂₀ heterocyclic group; a C₃-C₂₀ cycloalkyl group; the group consisting of a C₇-C₂₀ arylalkyl group and a C₈-C₂₀ arylalkenyl group, and also, these substituents may combine each other and form a ring, wherein the term ‘ring’ means a C₃-C₆₀ aliphatic ring or a C₆-C₆₀ aromatic ring or a C₂-C₆₀ heterocyclic ring or a fused ring formed by the combination of them, and includes a saturated or unsaturated ring.)}

In another embodiment of the invention, the present invention further provides an organic electric element characterized by comprising a first electrode; a second electrode; and an organic material layer; disposed between the first electrode and the second electrode, and comprising at least one hole transport layer and emitting layer, wherein the hole transport layer contains composition of two kinds of compounds having different structures of compounds represented by the Formula 1.

In another embodiment of the present invention, at least any one of the two kinds of compounds represented by the Formula 1 is one of the following Formulas 1-2, 1-3 and 1-4.

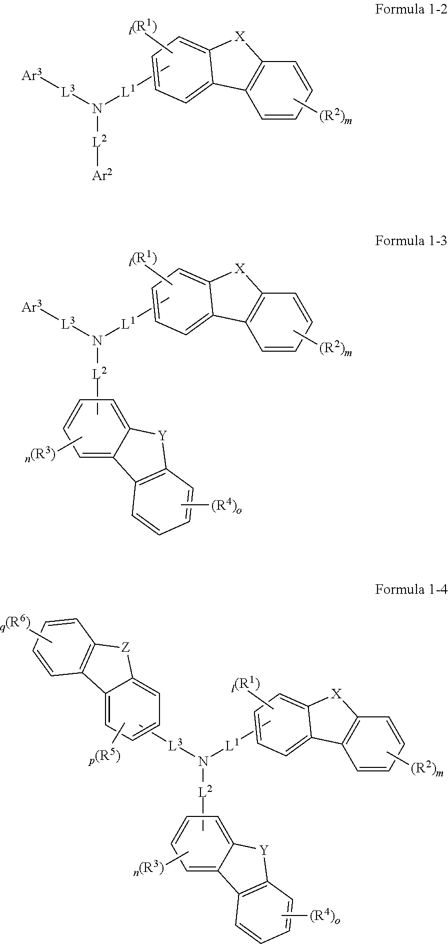

(In the Formula 1-2, 1-3 and 1-4, wherein Ar², Ar³, L¹, L² and L³ are the same as defined above, and X, Y and Z are S, O, CR′R″, and R′ and R″ are selected from the group consisting of a C₆-C₂₄ aryl group, a C₁-C₂₀ alkyl group, a C₂-C₂₀ alkenyl group and a C₁-C₂₀ alkoxy group and R′and R″ are may combine each other and form a spiro, and R¹, R², R³, R⁴, R⁵ and R⁶ are selected from the group consisting of deuterium, tritium, a cyano group, a nitro group, halogen, an aryl group, an alkenyl group, an alkylene group, an alkoxy group and an heterocyclic group, and a plurality of R¹ or plurality of R² or plurality of R³ or plurality of R⁴ or plurality of R⁵ or plurality of R⁵ or plurality of R⁶ may combine to each other to form a ring, and l, n and p are integer of 0 to 3, and m, o and q are an integer of 0 to 4.)

In another embodiment of the present invention, the Formula 1 is represented by any one of the following compounds.

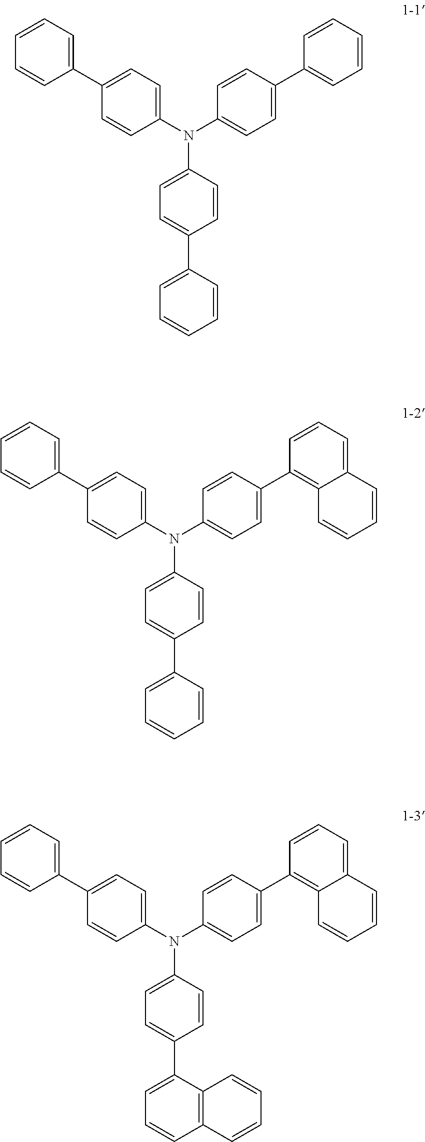 [SMILES: c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4cccc5ccccc45)cc3)cc2)cc1 ; c1ccc(-c2ccc(N(c3ccc(-c4cccc5ccccc45)cc3)c3ccc(-c4cccc5ccccc45)cc3)cc2)cc1]

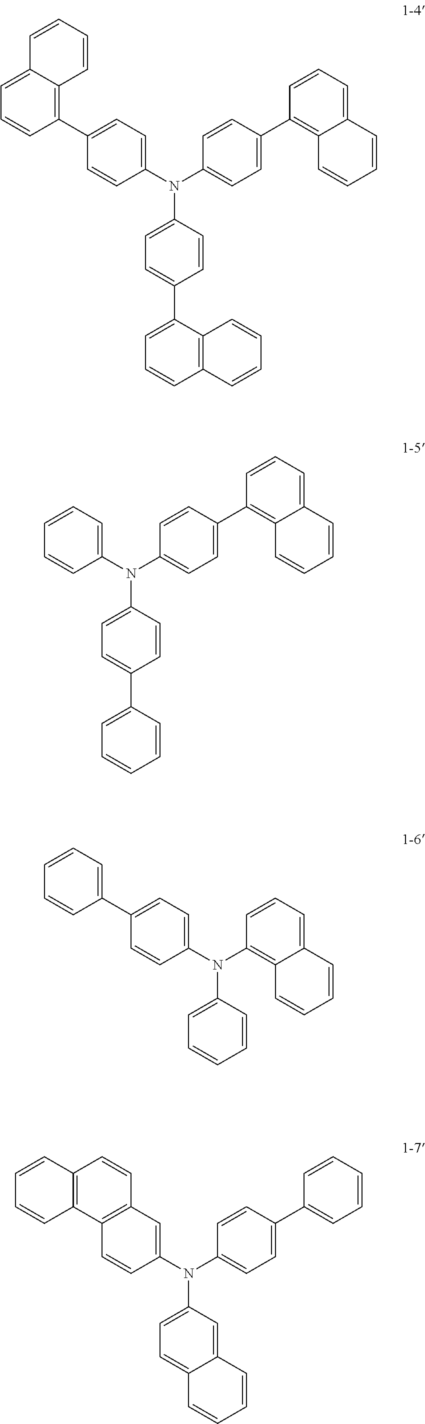 [SMILES: c1ccc2c(-c3ccc(N(c4ccc(-c5cccc6ccccc56)cc4)c4ccc(-c5cccc6ccccc56)cc4)cc3)cccc2c1 ; c1ccc(-c2ccc(N(c3ccccc3)c3ccc(-c4cccc5ccccc45)cc3)cc2)cc1 ; c1ccc(-c2ccc(N(c3ccccc3)c3cccc4ccccc34)cc2)cc1 ; c1ccc(-c2ccc(N(c3ccc4ccccc4c3)c3ccc4c(ccc5ccccc54)c3)cc2)cc1]

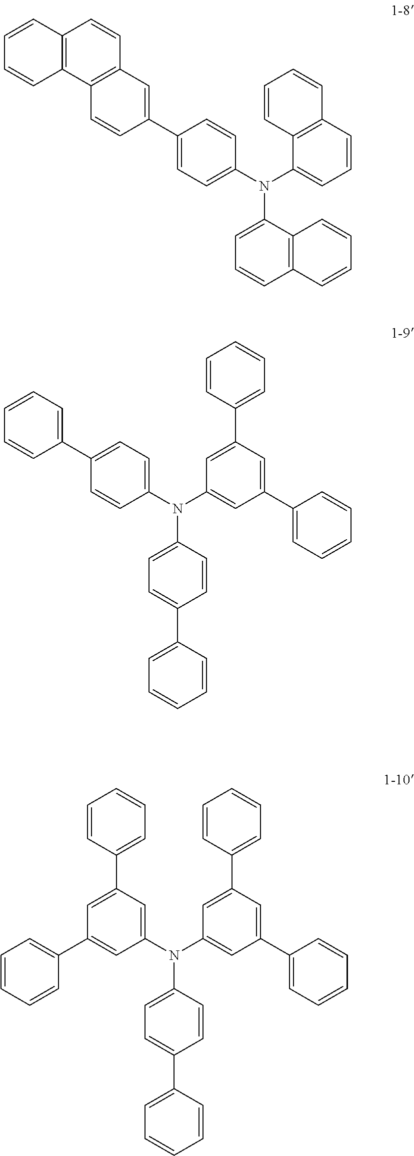 [SMILES: c1ccc2c(N(c3ccc(-c4ccc5c(ccc6ccccc65)c4)cc3)c3cccc4ccccc34)cccc2c1 ; c1ccc(-c2ccc(N(c3ccc(-c4ccccc4)cc3)c3cc(-c4ccccc4)cc(-c4ccccc4)c3)cc2)cc1 ; c1ccc(-c2ccc(N(c3cc(-c4ccccc4)cc(-c4ccccc4)c3)c3cc(-c4ccccc4)cc(-c4ccccc4)c3)cc2)cc1]

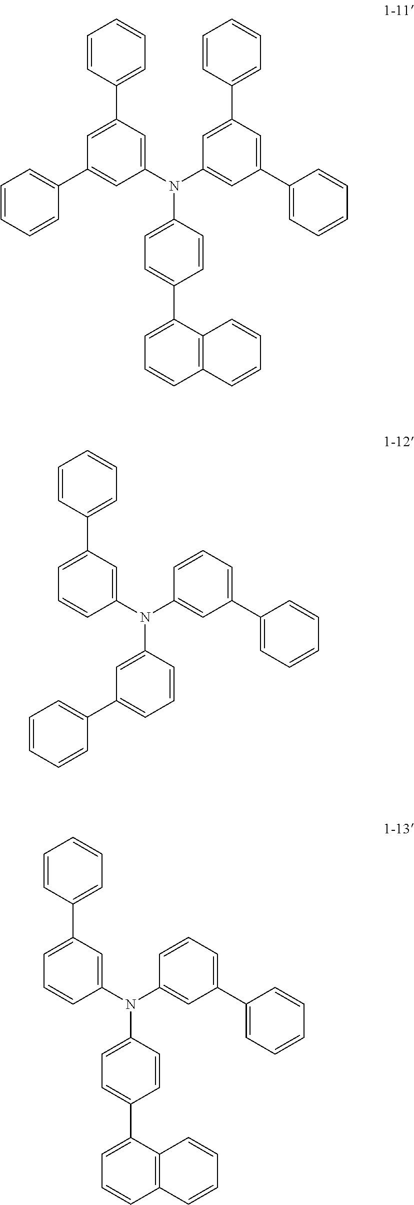 [SMILES: c1ccc(-c2cc(-c3ccccc3)cc(N(c3ccc(-c4cccc5ccccc45)cc3)c3cc(-c4ccccc4)cc(-c4ccccc4)c3)c2)cc1 ; c1ccc(-c2cccc(N(c3cccc(-c4ccccc4)c3)c3cccc(-c4ccccc4)c3)c2)cc1 ; c1ccc(-c2cccc(N(c3ccc(-c4cccc5ccccc45)cc3)c3cccc(-c4ccccc4)c3)c2)cc1]

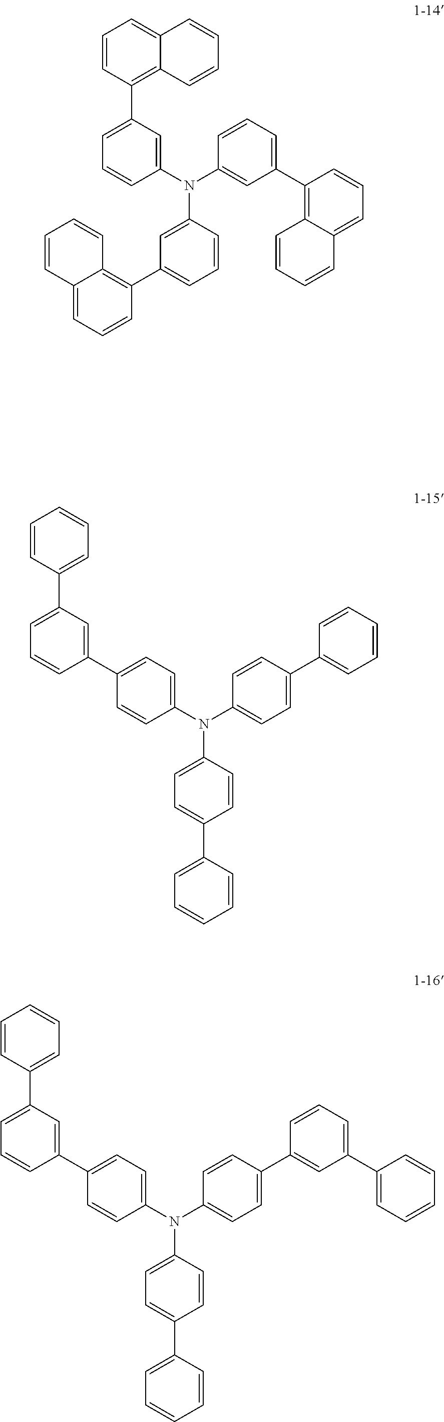 [SMILES: c1cc(-c2cccc3ccccc23)cc(N(c2cccc(-c3cccc4ccccc34)c2)c2cccc(-c3cccc4ccccc34)c2)c1 ; c1ccc(-c2ccc(N(c3ccc(-c4ccccc4)cc3)c3ccc(-c4cccc(-c5ccccc5)c4)cc3)cc2)cc1 ; c1ccc(-c2ccc(N(c3ccc(-c4cccc(-c5ccccc5)c4)cc3)c3ccc(-c4cccc(-c5ccccc5)c4)cc3)cc2)cc1]

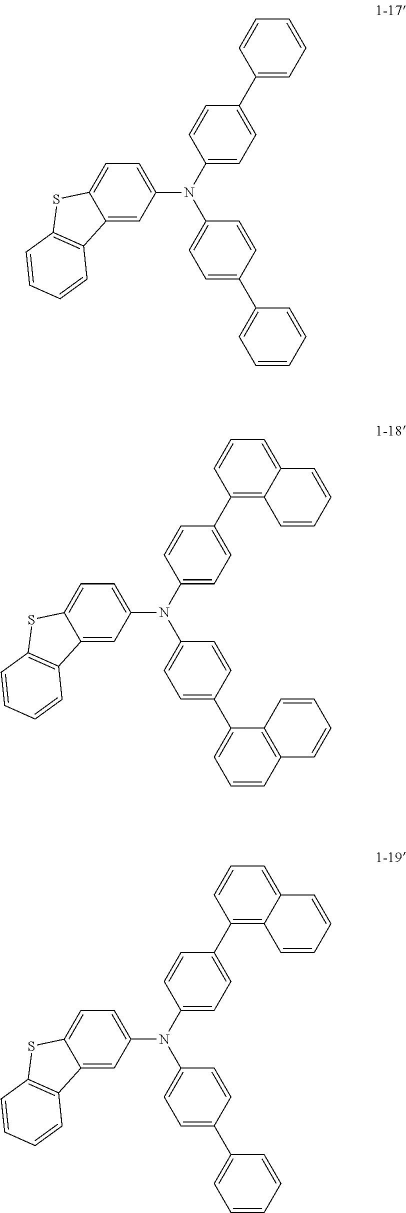 [SMILES: c1ccc(-c2ccc(N(c3ccc(-c4ccccc4)cc3)c3ccc4sc5ccccc5c4c3)cc2)cc1 ; c1ccc2c(-c3ccc(N(c4ccc(-c5cccc6ccccc56)cc4)c4ccc5sc6ccccc6c5c4)cc3)cccc2c1 ; c1ccc(-c2ccc(N(c3ccc(-c4cccc5ccccc45)cc3)c3ccc4sc5ccccc5c4c3)cc2)cc1]

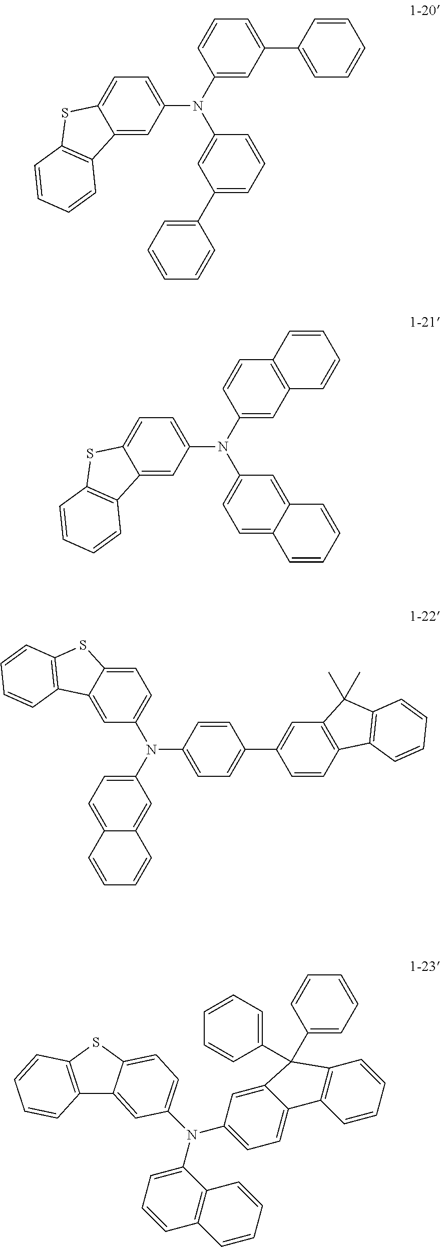 [SMILES: c1ccc(-c2cccc(N(c3cccc(-c4ccccc4)c3)c3ccc4sc5ccccc5c4c3)c2)cc1 ; c1ccc2cc(N(c3ccc4ccccc4c3)c3ccc4sc5ccccc5c4c3)ccc2c1 ; CC1(C)c2ccccc2-c2ccc(-c3ccc(N(c4ccc5ccccc5c4)c4ccc5sc6ccccc6c5c4)cc3)cc21 ; c1ccc(C2(c3ccccc3)c3ccccc3-c3ccc(N(c4ccc5sc6ccccc6c5c4)c4cccc5ccccc45)cc32)cc1]

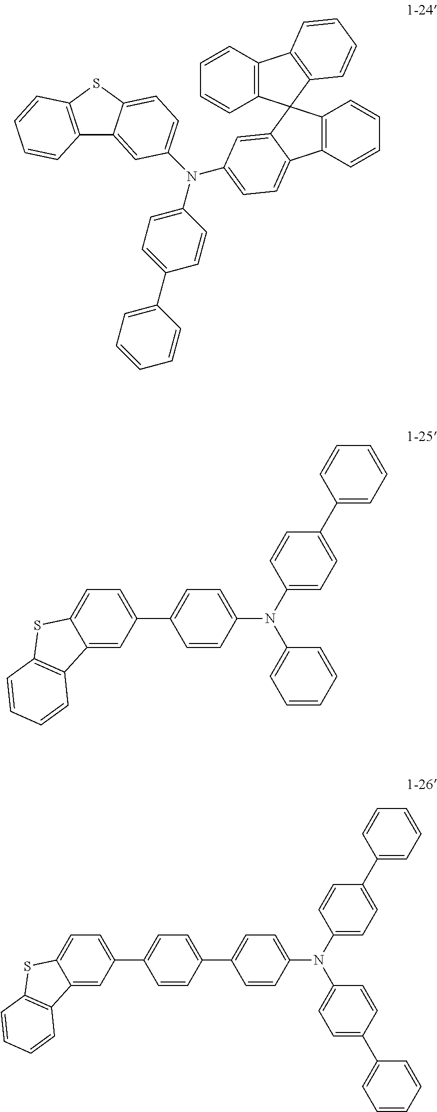 [SMILES: c1ccc(-c2ccc(N(c3ccc4c(c3)C3(c5ccccc5-c5ccccc53)c3ccccc3-4)c3ccc4sc5ccccc5c4c3)cc2)cc1 ; c1ccc(-c2ccc(N(c3ccccc3)c3ccc(-c4ccc5sc6ccccc6c5c4)cc3)cc2)cc1 ; c1ccc(-c2ccc(N(c3ccc(-c4ccccc4)cc3)c3ccc(-c4ccc(-c5ccc6sc7ccccc7c6c5)cc4)cc3)cc2)cc1]

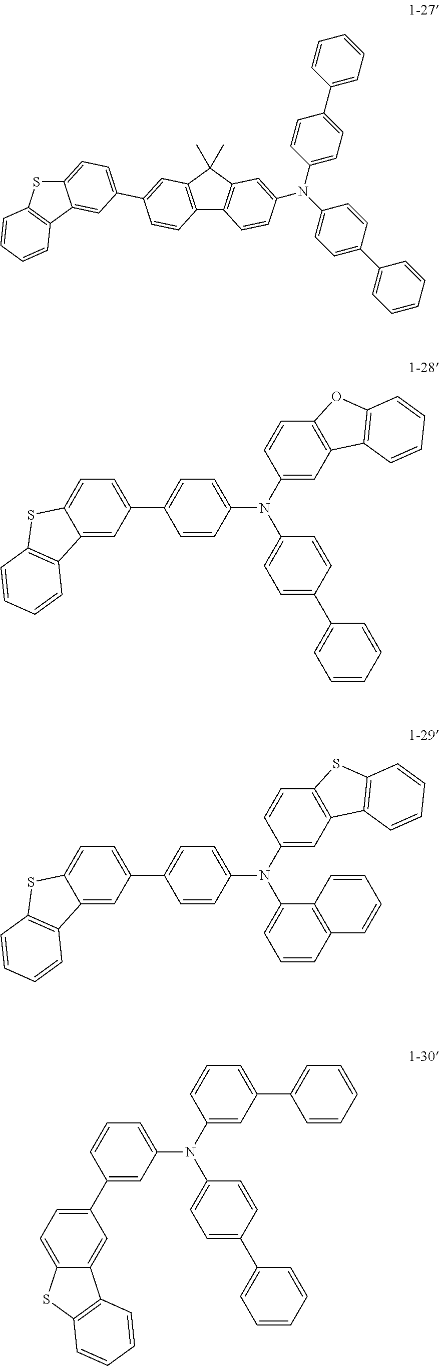 [SMILES: CC1(C)c2cc(-c3ccc4sc5ccccc5c4c3)ccc2-c2ccc(N(c3ccc(-c4ccccc4)cc3)c3ccc(-c4ccccc4)cc3)cc21 ; c1ccc(-c2ccc(N(c3ccc(-c4ccc5sc6ccccc6c5c4)cc3)c3ccc4oc5ccccc5c4c3)cc2)cc1 ; c1ccc2c(N(c3ccc(-c4ccc5sc6ccccc6c5c4)cc3)c3ccc4sc5ccccc5c4c3)cccc2c1 ; c1ccc(-c2ccc(N(c3cccc(-c4ccccc4)c3)c3cccc(-c4ccc5sc6ccccc6c5c4)c3)cc2)cc1]

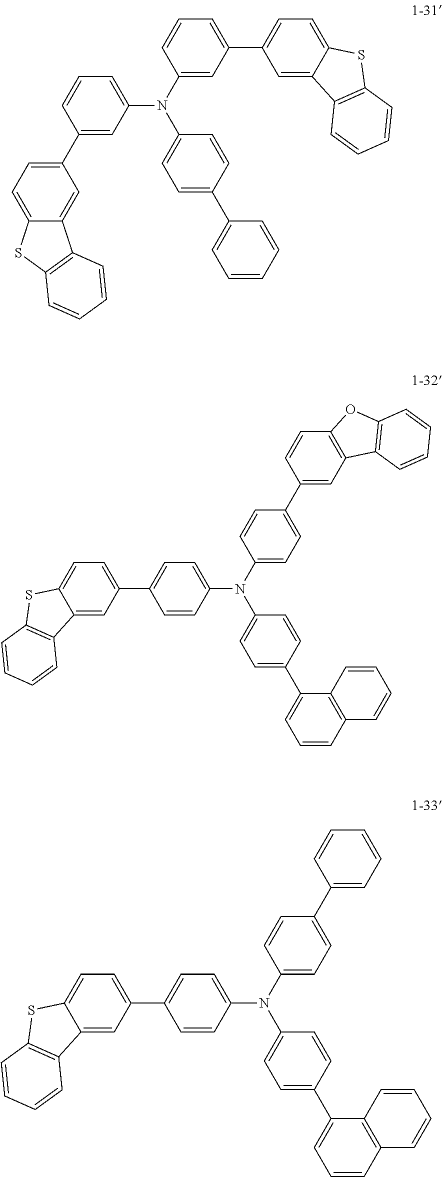 [SMILES: c1ccc(-c2ccc(N(c3cccc(-c4ccc5sc6ccccc6c5c4)c3)c3cccc(-c4ccc5sc6ccccc6c5c4)c3)cc2)cc1 ; c1ccc2c(-c3ccc(N(c4ccc(-c5ccc6oc7ccccc7c6c5)cc4)c4ccc(-c5ccc6sc7ccccc7c6c5)cc4)cc3)cccc2c1 ; c1ccc(-c2ccc(N(c3ccc(-c4ccc5sc6ccccc6c5c4)cc3)c3ccc(-c4cccc5ccccc45)cc3)cc2)cc1]

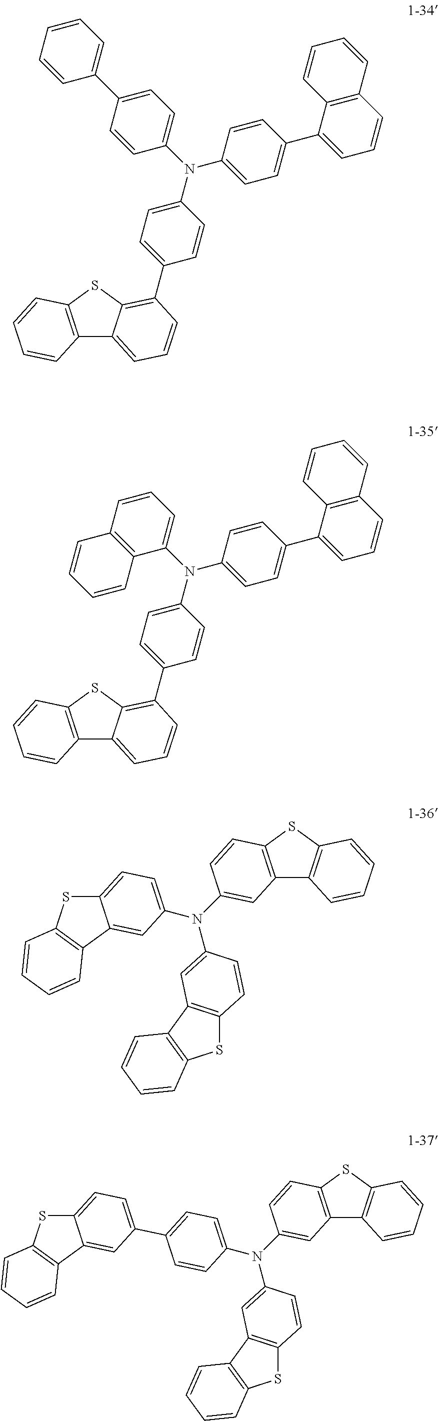 [SMILES: c1ccc(-c2ccc(N(c3ccc(-c4cccc5ccccc45)cc3)c3ccc(-c4cccc5c4sc4ccccc45)cc3)cc2)cc1 ; c1ccc2c(-c3ccc(N(c4ccc(-c5cccc6c5sc5ccccc56)cc4)c4cccc5ccccc45)cc3)cccc2c1 ; c1ccc2c(c1)sc1ccc(N(c3ccc4sc5ccccc5c4c3)c3ccc4sc5ccccc5c4c3)cc12 ; c1ccc2c(c1)sc1ccc(-c3ccc(N(c4ccc5sc6ccccc6c5c4)c4ccc5sc6ccccc6c5c4)cc3)cc12]

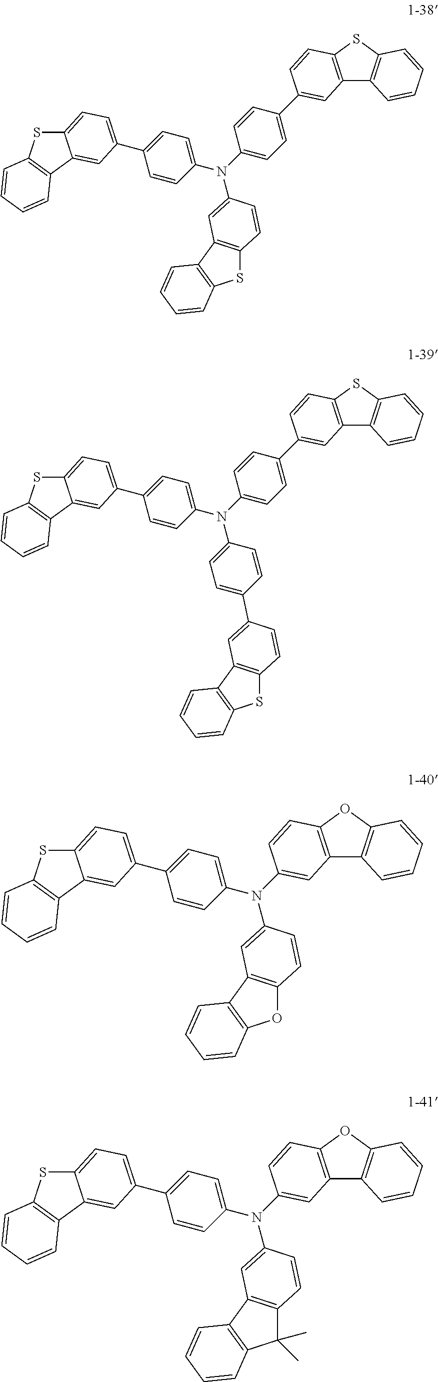 [SMILES: c1ccc2c(c1)sc1ccc(-c3ccc(N(c4ccc(-c5ccc6sc7ccccc7c6c5)cc4)c4ccc5sc6ccccc6c5c4)cc3)cc12 ; c1ccc2c(c1)sc1ccc(-c3ccc(N(c4ccc(-c5ccc6sc7ccccc7c6c5)cc4)c4ccc(-c5ccc6sc7ccccc7c6c5)cc4)cc3)cc12 ; c1ccc2c(c1)oc1ccc(N(c3ccc(-c4ccc5sc6ccccc6c5c4)cc3)c3ccc4oc5ccccc5c4c3)cc12 ; CC1(C)c2ccccc2-c2cc(N(c3ccc(-c4ccc5sc6ccccc6c5c4)cc3)c3ccc4oc5ccccc5c4c3)ccc21]

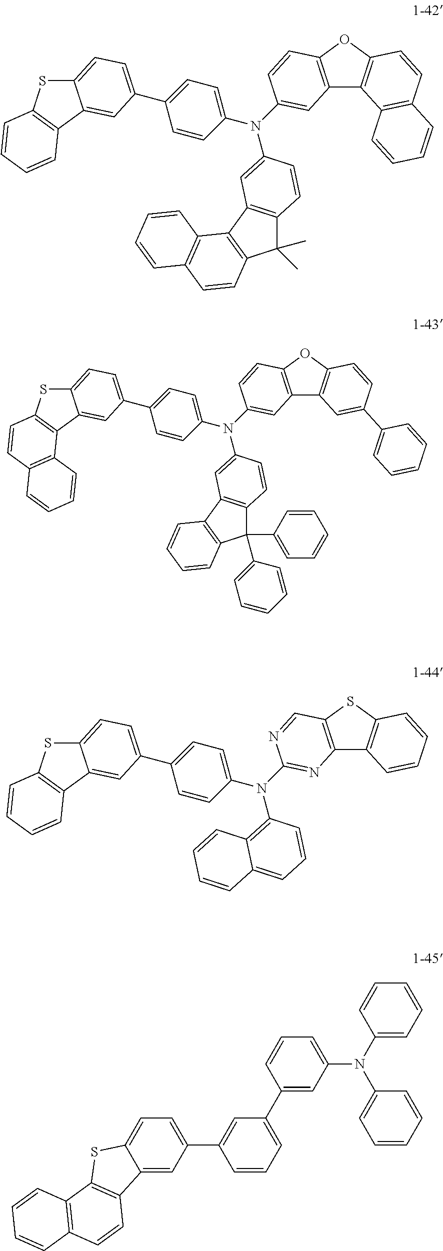 [SMILES: CC1(C)c2ccc(N(c3ccc(-c4ccc5sc6ccccc6c5c4)cc3)c3ccc4oc5ccc6ccccc6c5c4c3)cc2-c2c1ccc1ccccc21 ; c1ccc(-c2ccc3oc4ccc(N(c5ccc(-c6ccc7sc8ccc9ccccc9c8c7c6)cc5)c5ccc6c(c5)-c5ccccc5C6(c5ccccc5)c5ccccc5)cc4c3c2)cc1 ; c1ccc2c(N(c3ccc(-c4ccc5sc6ccccc6c5c4)cc3)c3ncc4sc5ccccc5c4n3)cccc2c1 ; c1ccc(N(c2ccccc2)c2cccc(-c3cccc(-c4ccc5sc6c7ccccc7ccc6c5c4)c3)c2)cc1]

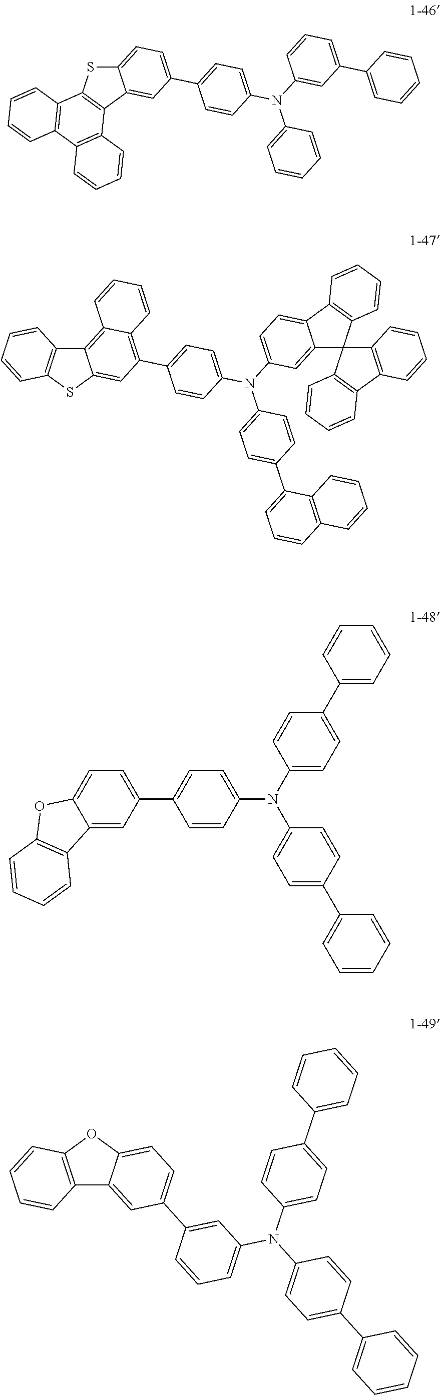 [SMILES: c1ccc(-c2cccc(N(c3ccccc3)c3ccc(-c4ccc5sc6c7ccccc7c7ccccc7c6c5c4)cc3)c2)cc1 ; c1ccc2c(c1)-c1ccccc1C21c2ccccc2-c2ccc(N(c3ccc(-c4cccc5ccccc45)cc3)c3ccc(-c4cc5sc6ccccc6c5c5ccccc45)cc3)cc21 ; c1ccc(-c2ccc(N(c3ccc(-c4ccccc4)cc3)c3ccc(-c4ccc5oc6ccccc6c5c4)cc3)cc2)cc1 ; c1ccc(-c2ccc(N(c3ccc(-c4ccccc4)cc3)c3cccc(-c4ccc5oc6ccccc6c5c4)c3)cc2)cc1]

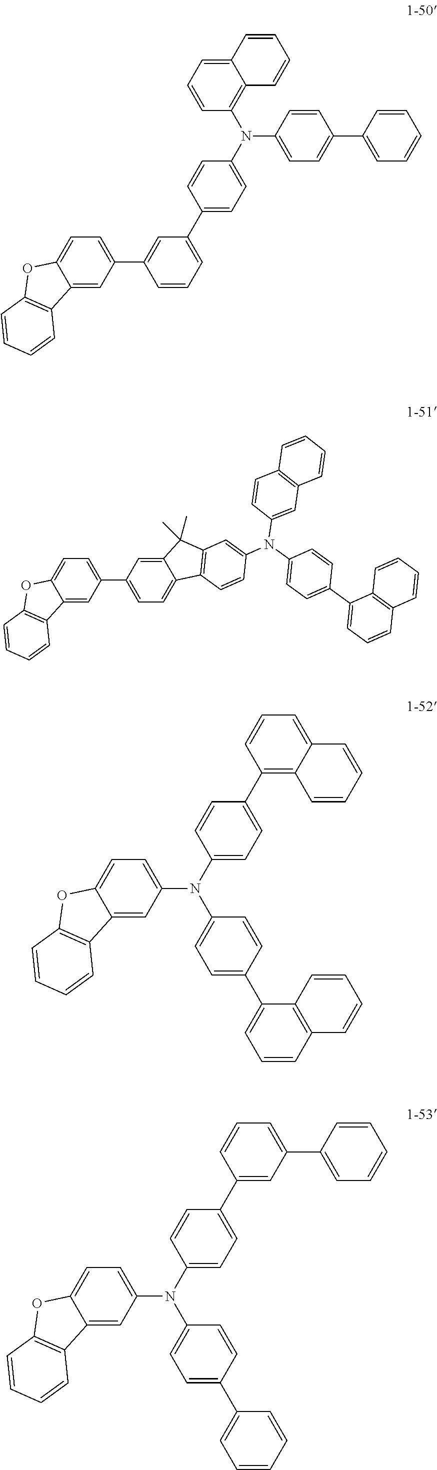 [SMILES: c1ccc(-c2ccc(N(c3ccc(-c4cccc(-c5ccc6oc7ccccc7c6c5)c4)cc3)c3cccc4ccccc34)cc2)cc1 ; CC1(C)c2cc(-c3ccc4oc5ccccc5c4c3)ccc2-c2ccc(N(c3ccc(-c4cccc5ccccc45)cc3)c3ccc4ccccc4c3)cc21 ; c1ccc2c(-c3ccc(N(c4ccc(-c5cccc6ccccc56)cc4)c4ccc5oc6ccccc6c5c4)cc3)cccc2c1 ; c1ccc(-c2ccc(N(c3ccc(-c4cccc(-c5ccccc5)c4)cc3)c3ccc4oc5ccccc5c4c3)cc2)cc1]

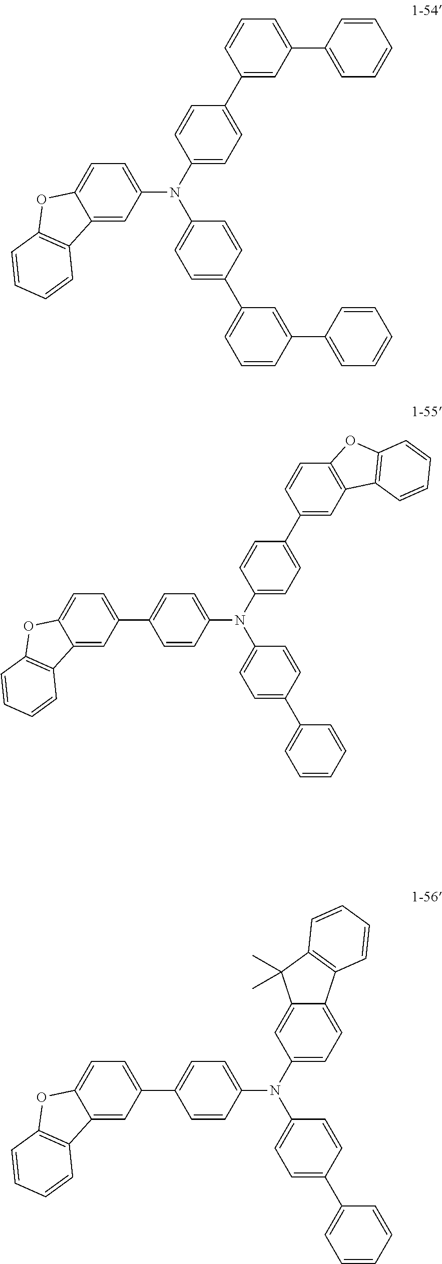 [SMILES: c1ccc(-c2cccc(-c3ccc(N(c4ccc(-c5cccc(-c6ccccc6)c5)cc4)c4ccc5oc6ccccc6c5c4)cc3)c2)cc1 ; c1ccc(-c2ccc(N(c3ccc(-c4ccc5oc6ccccc6c5c4)cc3)c3ccc(-c4ccc5oc6ccccc6c5c4)cc3)cc2)cc1 ; CC1(C)c2ccccc2-c2ccc(N(c3ccc(-c4ccccc4)cc3)c3ccc(-c4ccc5oc6ccccc6c5c4)cc3)cc21]

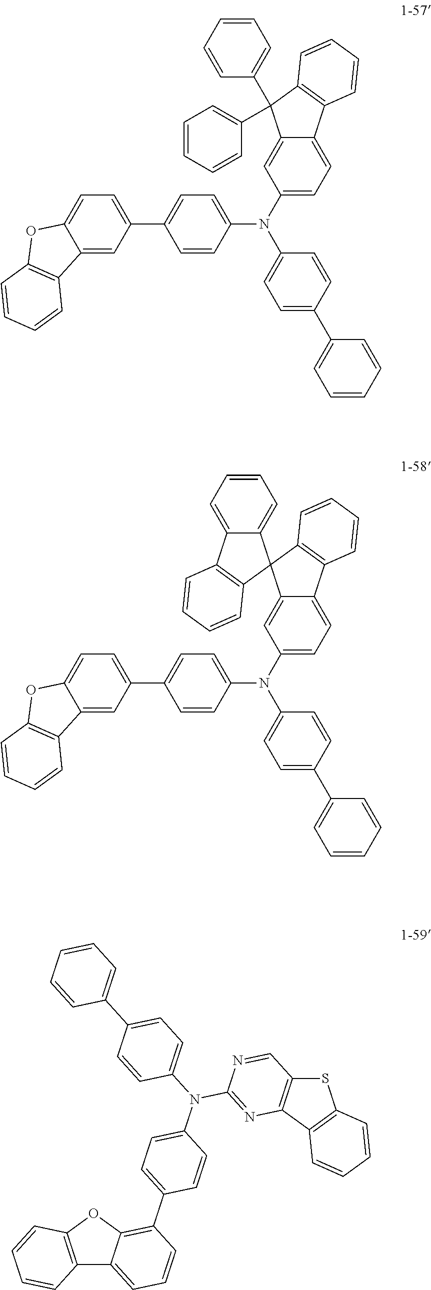 [SMILES: c1ccc(-c2ccc(N(c3ccc(-c4ccc5oc6ccccc6c5c4)cc3)c3ccc4c(c3)C(c3ccccc3)(c3ccccc3)c3ccccc3-4)cc2)cc1 ; c1ccc(-c2ccc(N(c3ccc(-c4ccc5oc6ccccc6c5c4)cc3)c3ccc4c(c3)C3(c5ccccc5-c5ccccc53)c3ccccc3-4)cc2)cc1 ; c1ccc(-c2ccc(N(c3ccc(-c4cccc5c4oc4ccccc45)cc3)c3ncc4sc5ccccc5c4n3)cc2)cc1]

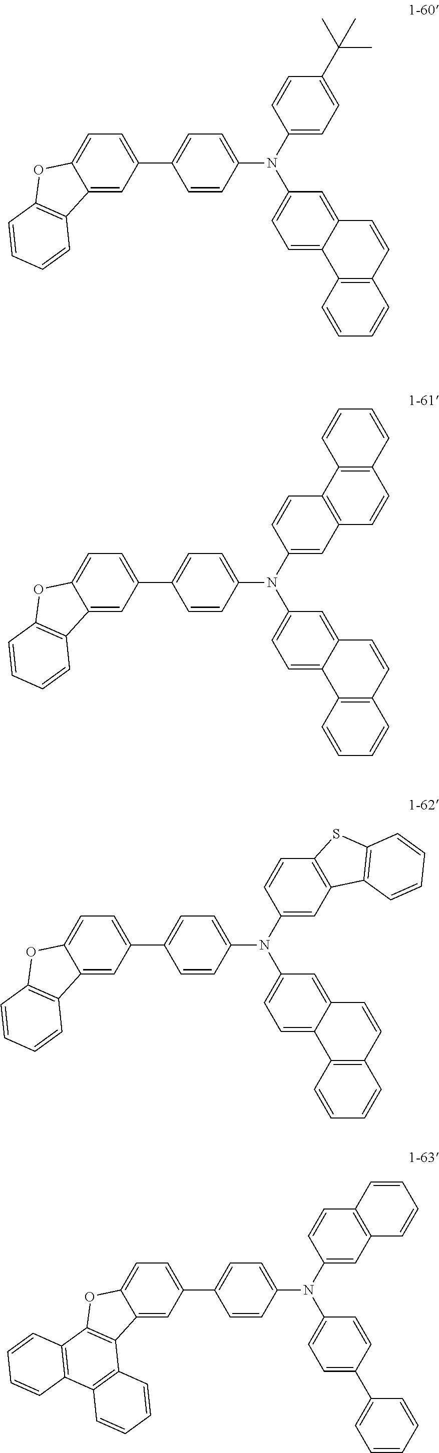 [SMILES: CC(C)(C)c1ccc(N(c2ccc(-c3ccc4oc5ccccc5c4c3)cc2)c2ccc3c(ccc4ccccc43)c2)cc1 ; c1ccc2c(c1)ccc1cc(N(c3ccc(-c4ccc5oc6ccccc6c5c4)cc3)c3ccc4c(ccc5ccccc54)c3)ccc12 ; c1ccc2c(c1)ccc1cc(N(c3ccc(-c4ccc5oc6ccccc6c5c4)cc3)c3ccc4sc5ccccc5c4c3)ccc12 ; c1ccc(-c2ccc(N(c3ccc(-c4ccc5oc6c7ccccc7c7ccccc7c6c5c4)cc3)c3ccc4ccccc4c3)cc2)cc1]

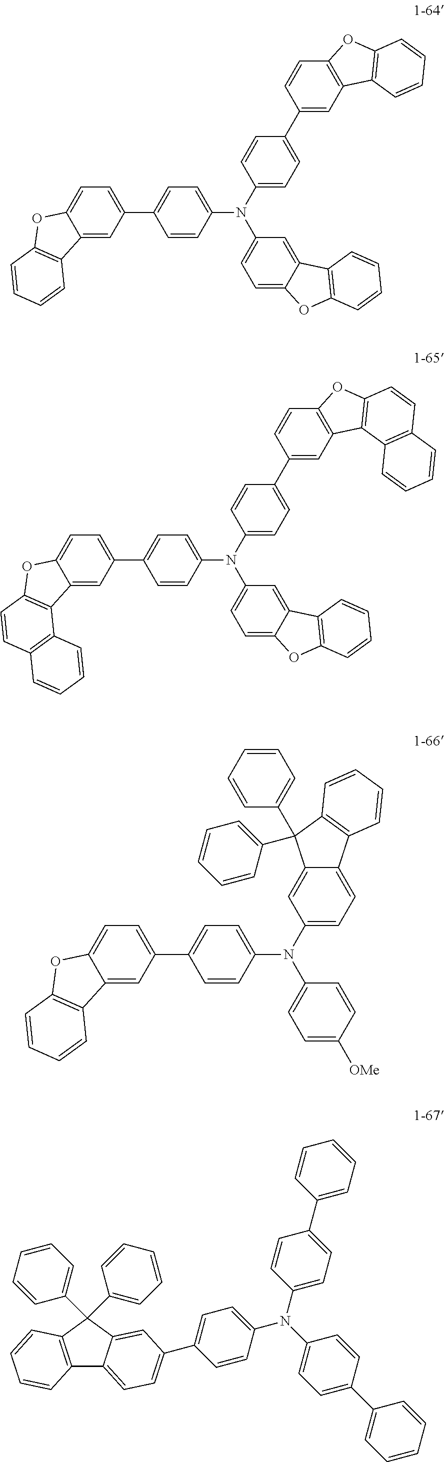 [SMILES: c1ccc2c(c1)oc1ccc(-c3ccc(N(c4ccc(-c5ccc6oc7ccccc7c6c5)cc4)c4ccc5oc6ccccc6c5c4)cc3)cc12 ; c1ccc2c(c1)ccc1oc3ccc(-c4ccc(N(c5ccc(-c6ccc7oc8ccc9ccccc9c8c7c6)cc5)c5ccc6oc7ccccc7c6c5)cc4)cc3c12 ; COc1ccc(N(c2ccc(-c3ccc4oc5ccccc5c4c3)cc2)c2ccc3c(c2)C(c2ccccc2)(c2ccccc2)c2ccccc2-3)cc1 ; c1ccc(-c2ccc(N(c3ccc(-c4ccccc4)cc3)c3ccc(-c4ccc5c(c4)C(c4ccccc4)(c4ccccc4)c4ccccc4-5)cc3)cc2)cc1]

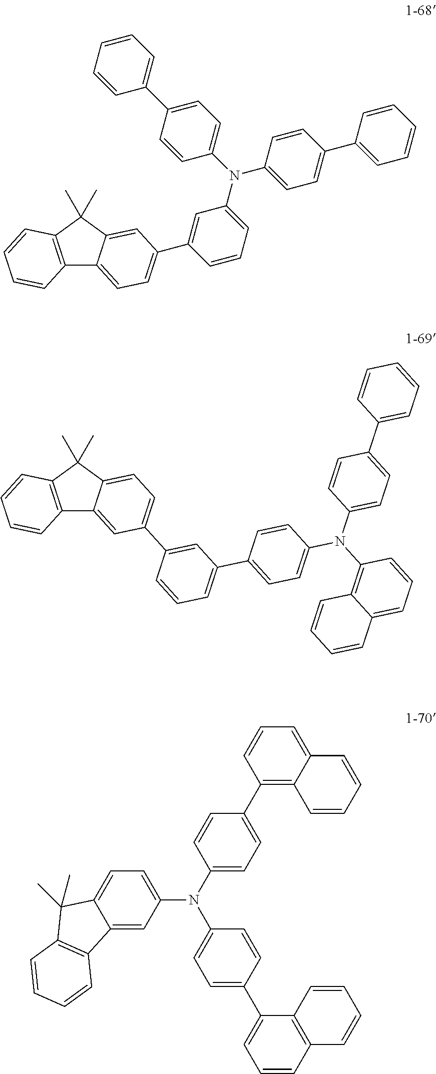 [SMILES: CC1(C)c2ccccc2-c2ccc(-c3cccc(N(c4ccc(-c5ccccc5)cc4)c4ccc(-c5ccccc5)cc4)c3)cc21 ; CC1(C)c2ccccc2-c2cc(-c3cccc(-c4ccc(N(c5ccc(-c6ccccc6)cc5)c5cccc6ccccc56)cc4)c3)ccc21 ; CC1(C)c2ccccc2-c2cc(N(c3ccc(-c4cccc5ccccc45)cc3)c3ccc(-c4cccc5ccccc45)cc3)ccc21]

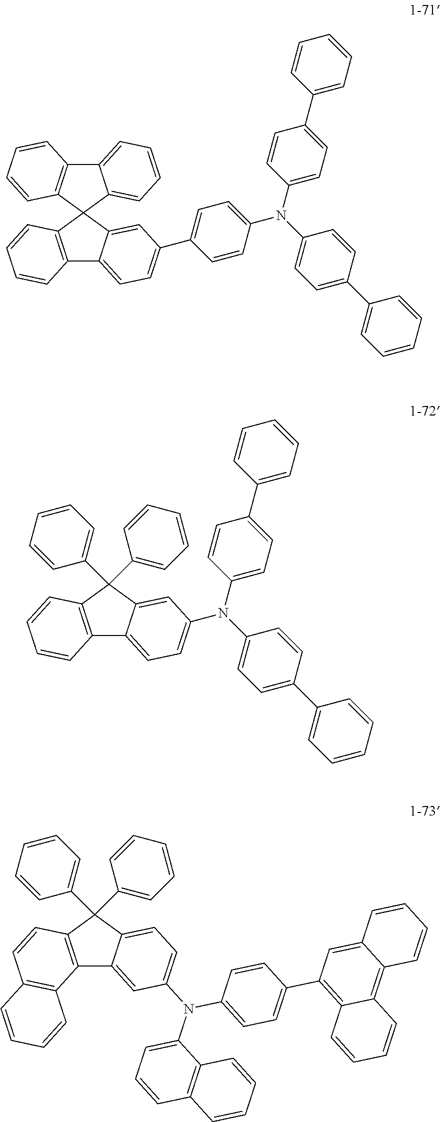 [SMILES: c1ccc(-c2ccc(N(c3ccc(-c4ccccc4)cc3)c3ccc(-c4ccc5c(c4)C4(c6ccccc6-c6ccccc64)c4ccccc4-5)cc3)cc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1 ; c1ccc(C2(c3ccccc3)c3ccc(N(c4ccc(-c5cc6ccccc6c6ccccc56)cc4)c4cccc5ccccc45)cc3-c3c2ccc2ccccc32)cc1]

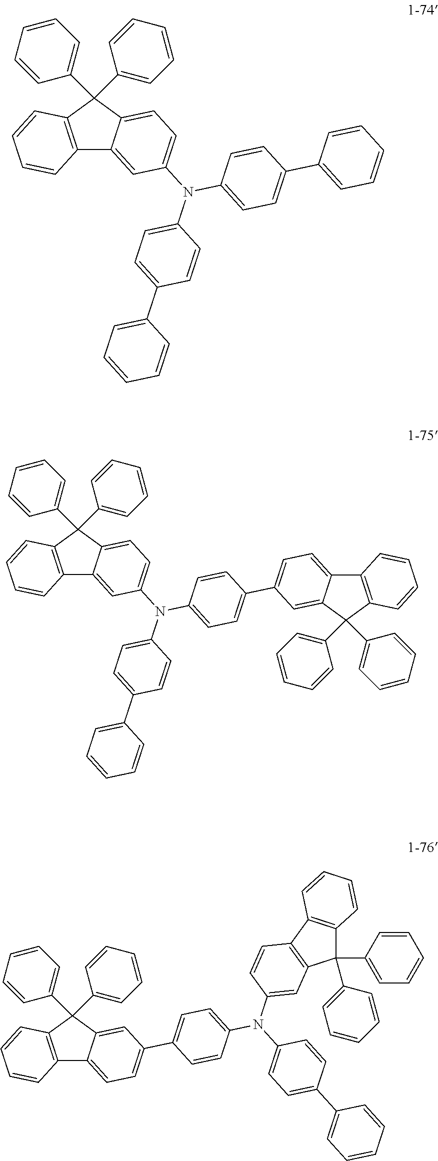 [SMILES: c1ccc(-c2ccc(N(c3ccc(-c4ccccc4)cc3)c3ccc4c(c3)-c3ccccc3C4(c3ccccc3)c3ccccc3)cc2)cc1 ; c1ccc(-c2ccc(N(c3ccc(-c4ccc5c(c4)C(c4ccccc4)(c4ccccc4)c4ccccc4-5)cc3)c3ccc4c(c3)-c3ccccc3C4(c3ccccc3)c3ccccc3)cc2)cc1 ; c1ccc(-c2ccc(N(c3ccc(-c4ccc5c(c4)C(c4ccccc4)(c4ccccc4)c4ccccc4-5)cc3)c3ccc4c(c3)C(c3ccccc3)(c3ccccc3)c3ccccc3-4)cc2)cc1]

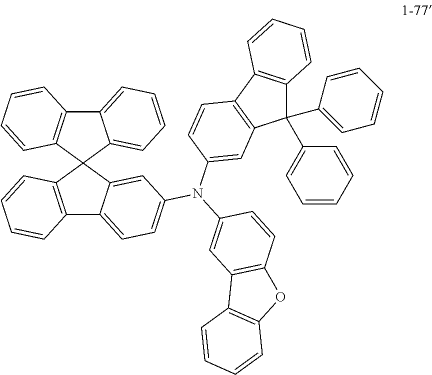 [SMILES: c1ccc(C2(c3ccccc3)c3ccccc3-c3ccc(N(c4ccc5c(c4)C4(c6ccccc6-c6ccccc64)c4ccccc4-5)c4ccc5oc6ccccc6c5c4)cc32)cc1]

In another embodiment of the present invention, Ar¹, Ar², Ar³ of two kinds of compounds represented by the Formula 1 are all C₆ to C₂₄ aryl groups.

In another embodiment of the present invention, Ar¹, Ar² and Ar³ of one of two compounds represented by the Formula 1 are all C₆ to C₂₄ aryl groups; and at least one of Ar¹, Ar² and Ar³ of the remaining one kind of compound are dibenzothiophene or dibenzofuran.

In another embodiment of the present invention, at least one of Ar¹, Ar², Ar³ of two compounds represented by the Formula 1 is dibenzothiophene or dibenzofuran.

In another embodiment of the present invention, the mixing ratio of any one of two compounds having different structures represented by the Formula 1 is 10%˜90%.

In an embodiment of another aspect of the present invention, when two compounds having different structures represented by the Formula 1 are mixed, the mixing ratio is at least one of 5:5 or 6:4 or 7:3 or 8:2 or 9:1.

In another embodiment presented in an example of the present invention, the mixture of two compounds having different structures represented by the Formula 1 further comprises one or more compounds represented by the Formula 1.

Moreover, an emitting auxiliary layer using compounds represented by the Formula 1 is between the emitting layer and the hole transport layer using the mixture of two compounds having different structures represented by the Formula 1, and the light efficiency improving layer is formed on at least one side opposite to the organic material layer among one side of the first electrode and the second electrode, wherein the organic material layer is formed by one of a spin coating process, a nozzle printing process, an inkjet printing process, a slot coating process, a dip coating process or a roll-to-roll process.

The present invention provides an electronic device comprising a display device and the control unit driving the display device including the organic electric element of various examples described above. Furthermore, the organic electric element may be applied at least one of an organic light emitting diode (OLED), an organic solar cell, an organic photo conductor, an organic transistor or a device for monochromic or white illumination.

Hereinafter, synthesis examples of the compound represented by Formula 1 comprised the organic electric element of the present invention and preparation examples of the organic electric element of the present invention will be described in detail by way of example. However, the following examples are only for illustrative purposes and are not intended to limit the following examples of the invention.

### Synthesis Example

The final product represented by Formula 1 according to the present invention can be synthesized by reaction between Sub 1 and Sub 2 as illustrated in the following Reaction Scheme 1.

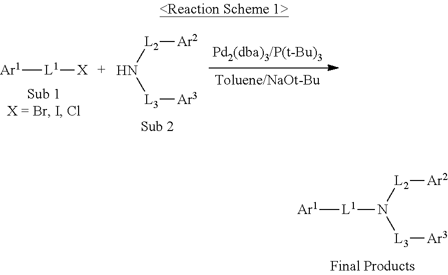

### Sub 1

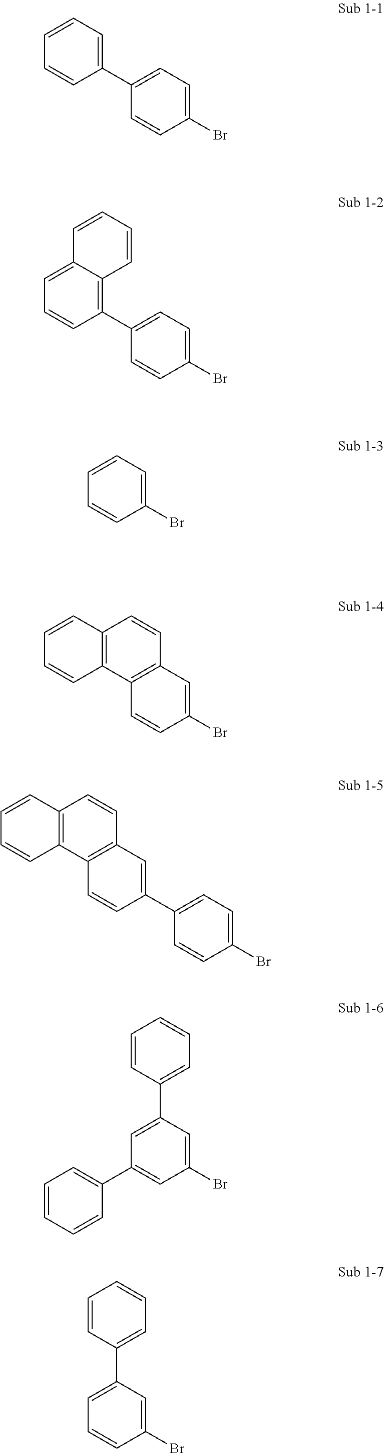

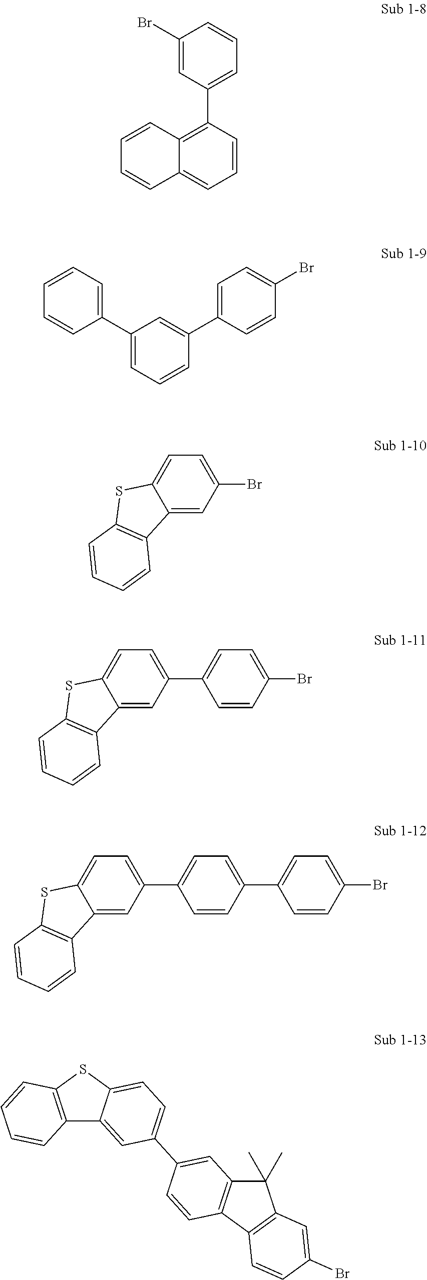 [SMILES: Brc1cccc(-c2cccc3ccccc23)c1 ; Brc1ccc(-c2cccc(-c3ccccc3)c2)cc1 ; Brc1ccc2sc3ccccc3c2c1 ; Brc1ccc(-c2ccc3sc4ccccc4c3c2)cc1 ; Brc1ccc(-c2ccc(-c3ccc4sc5ccccc5c4c3)cc2)cc1 ; CC1(C)c2cc(Br)ccc2-c2ccc(-c3ccc4sc5ccccc5c4c3)cc21]

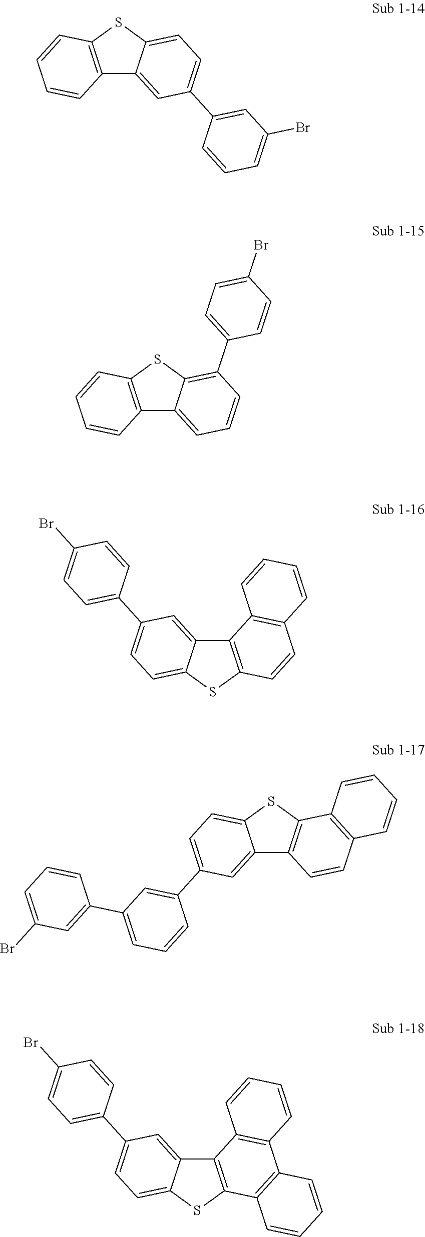 [SMILES: Brc1cccc(-c2ccc3sc4ccccc4c3c2)c1 ; Brc1ccc(-c2cccc3c2sc2ccccc23)cc1 ; Brc1ccc(-c2ccc3sc4ccc5ccccc5c4c3c2)cc1 ; Brc1cccc(-c2cccc(-c3ccc4sc5c6ccccc6ccc5c4c3)c2)c1 ; Brc1ccc(-c2ccc3sc4c5ccccc5c5ccccc5c4c3c2)cc1]

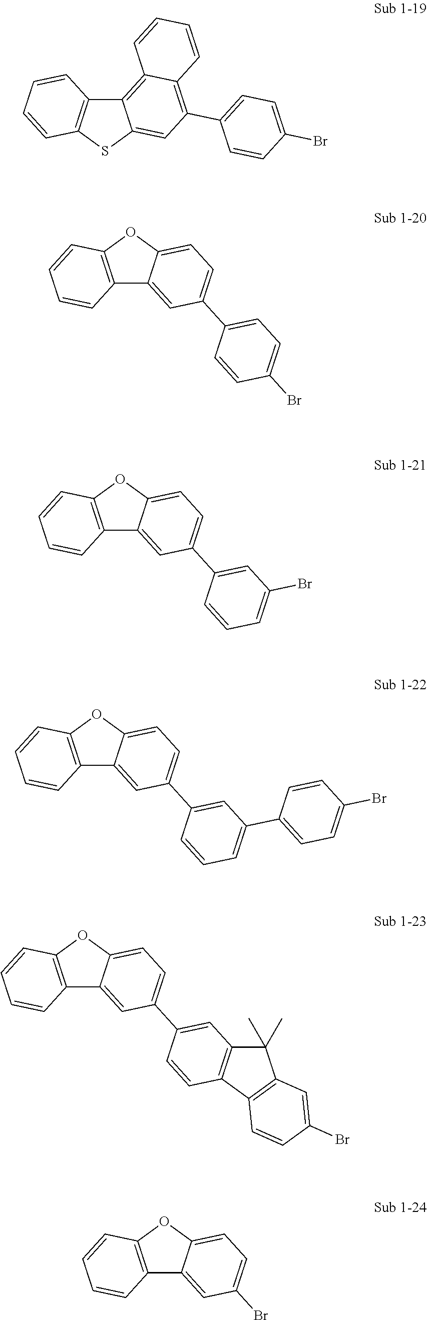 [SMILES: Brc1ccc(-c2cc3sc4ccccc4c3c3ccccc23)cc1 ; Brc1ccc(-c2ccc3oc4ccccc4c3c2)cc1 ; Brc1cccc(-c2ccc3oc4ccccc4c3c2)c1 ; Brc1ccc(-c2cccc(-c3ccc4oc5ccccc5c4c3)c2)cc1 ; CC1(C)c2cc(Br)ccc2-c2ccc(-c3ccc4oc5ccccc5c4c3)cc21 ; Brc1ccc2oc3ccccc3c2c1]

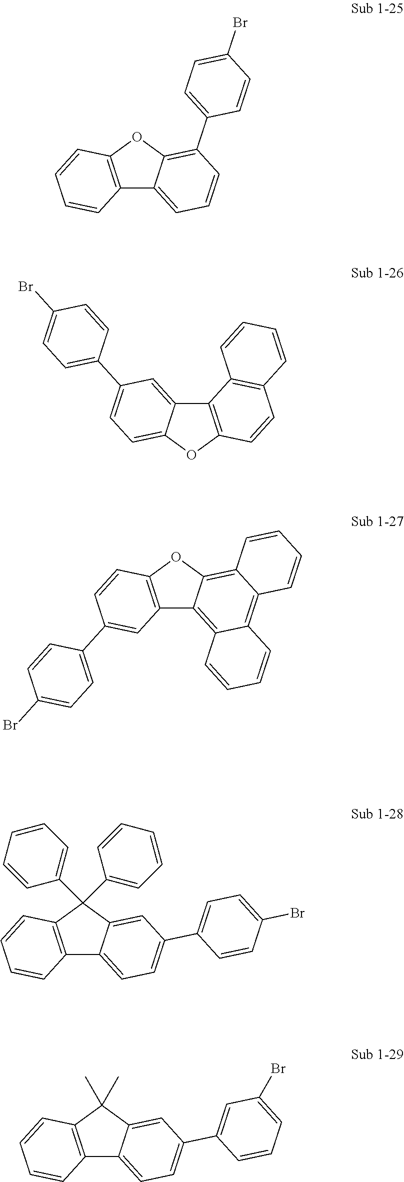 [SMILES: Brc1ccc(-c2cccc3c2oc2ccccc23)cc1 ; Brc1ccc(-c2ccc3oc4ccc5ccccc5c4c3c2)cc1 ; Brc1ccc(-c2ccc3oc4c5ccccc5c5ccccc5c4c3c2)cc1 ; Brc1ccc(-c2ccc3c(c2)C(c2ccccc2)(c2ccccc2)c2ccccc2-3)cc1 ; CC1(C)c2ccccc2-c2ccc(-c3cccc(Br)c3)cc21]

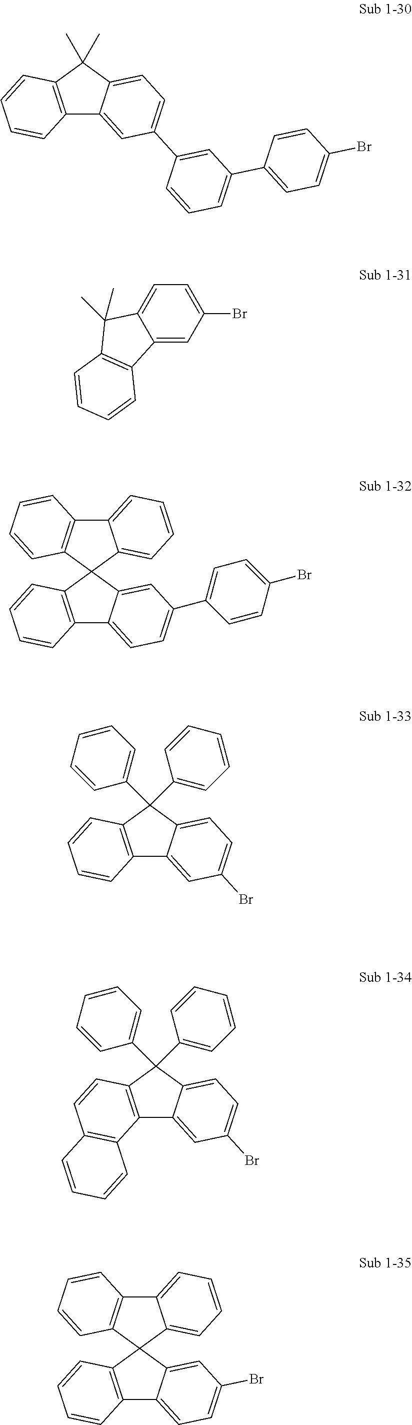 [SMILES: CC1(C)c2ccccc2-c2cc(-c3cccc(-c4ccc(Br)cc4)c3)ccc21 ; CC1(C)c2ccccc2-c2cc(Br)ccc21 ; Brc1ccc(-c2ccc3c(c2)C2(c4ccccc4-c4ccccc42)c2ccccc2-3)cc1 ; Brc1ccc2c(c1)-c1ccccc1C2(c1ccccc1)c1ccccc1 ; Brc1ccc2c(c1)-c1c(ccc3ccccc13)C2(c1ccccc1)c1ccccc1 ; Brc1ccc2c(c1)C1(c3ccccc3-c3ccccc31)c1ccccc1-2]

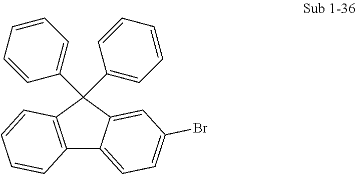 [SMILES: Brc1ccc2c(c1)C(c1ccccc1)(c1ccccc1)c1ccccc1-2]

### Synthesis Example of Sub 2

Sub 2 of Reaction Scheme 1 can be synthesized according to, but not limited to, the reaction path of the following Reaction Scheme 2 or the following Reaction Scheme 3.

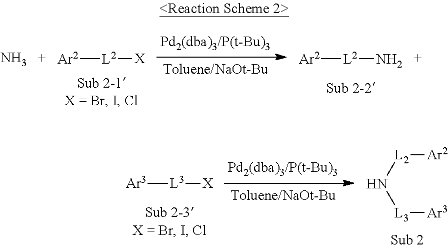

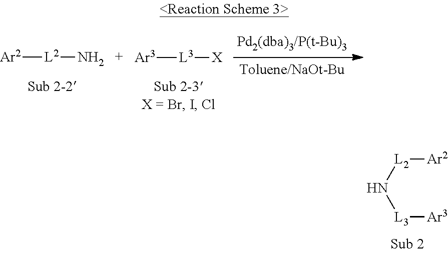

### Examples of Sub 2-1

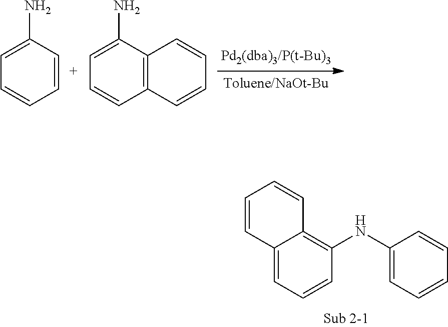

After Aniline (15 g, 161.1 mmol), 1-bromonaphthalene (36.7 g, 177.2 mmol), Pd₂(dba)₃ (7.37 g, 8.05 mmol), P(t-Bu)₃ (3.26 g, 16.1 mmol), NaOt-Bu(51.08 g, 531.5 mmol), and toluene (1690 mL) are added in a round bottom flask, stirring at 100° C. When the reaction is complete, the product was extracted with CH₂Cl₂ and water. The organic layer was dried over MgSO₄ and concentrated, and then the product was separated by a silicagel column chromatography and recrystallized to obtain 25.4 g of product Sub 2-1 (yield: 72%).

### Example of Sub 2-26

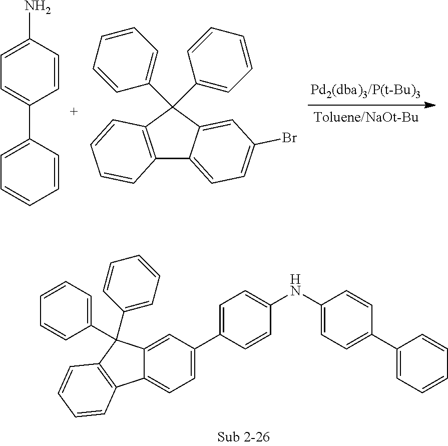

[1,1′-biphenyl]-4-amine (15 g, 88.6 mmol), 2-(4-bromophenyl)-9,9-diphenyl-9H-fluorene (46.2 g, 97.5 mmol), Pd₂(dba)₃ (4.06 g, 4.43 mmol), P(t-Bu)₃ (1.8 g, 8.86 mmol), NaOt-Bu (390 g, 292.5 mmol) and toluene (931 mL) were added in a round bottom flask, and the same procedure as described in the synthesis method of Sub 2-1 above was carried out to obtain 34.9 g of Sub 2-26 (yield: 70%).

### Example of Sub 2-40

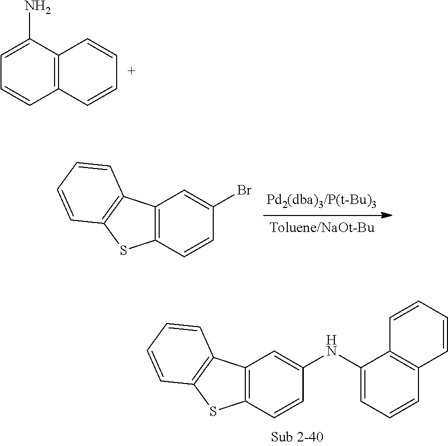 [SMILES: Nc1cccc2ccccc12 ; Brc1ccc2sc3ccccc3c2c1 ; c1ccc2c(Nc3ccc4sc5ccccc5c4c3)cccc2c1]

Naphthalen-1-amine (15 g, 104.8 mmol), 2-bromodibenzo[b,d]thiophene (30.3 g, 115.2 mmol), Pd₂(dba)₃ (4.8 g, 5.24 mmol), P(t-Bu)₃ (2.12 g, 10.48 mmol), NaOt-Bu (460.9 g, 345.7 mmol), and toluene (1100 mL) were added in a round bottom flask, and the same procedure as described in the synthesis method of Sub 2-1 above was carried out to obtain 24.9 g of Sub 2-40 (yield: 73%).

### Example of Sub 2-51

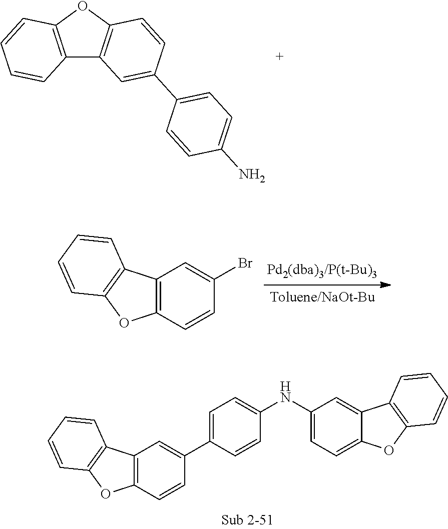 [SMILES: Nc1ccc(-c2ccc3oc4ccccc4c3c2)cc1 ; "+" ; Brc1ccc2oc3ccccc3c2c1 ; c1ccc2c(c1)oc1ccc(Nc3ccc(-c4ccc5oc6ccccc6c5c4)cc3)cc12]

4-(dibenzo[b,d]furan-2-yl)aniline (15 g, 57.85 mmol), 2-bromodibenzo[b,d]furan (15.7 g, 63.63 mmol), Pd₂(dba)₃ (2.65 g, 2.89 mmol), P(t-Bu)₃ (1.17 g, 5.78 mmol), NaOt-Bu (254.5 g, 190.9 mmol), toluene (607 mL) were added in a round bottom flask, and the same procedure as described in the synthesis method of Sub 2-1 was carried out to obtain 17.2 g of Sub 2-51 (yield: 70%).

The following Sub 2-1 to Sub 2-52 were synthesized with the same procedure as described in the synthesis method, and Sub 2 cannot be limited to the followings.

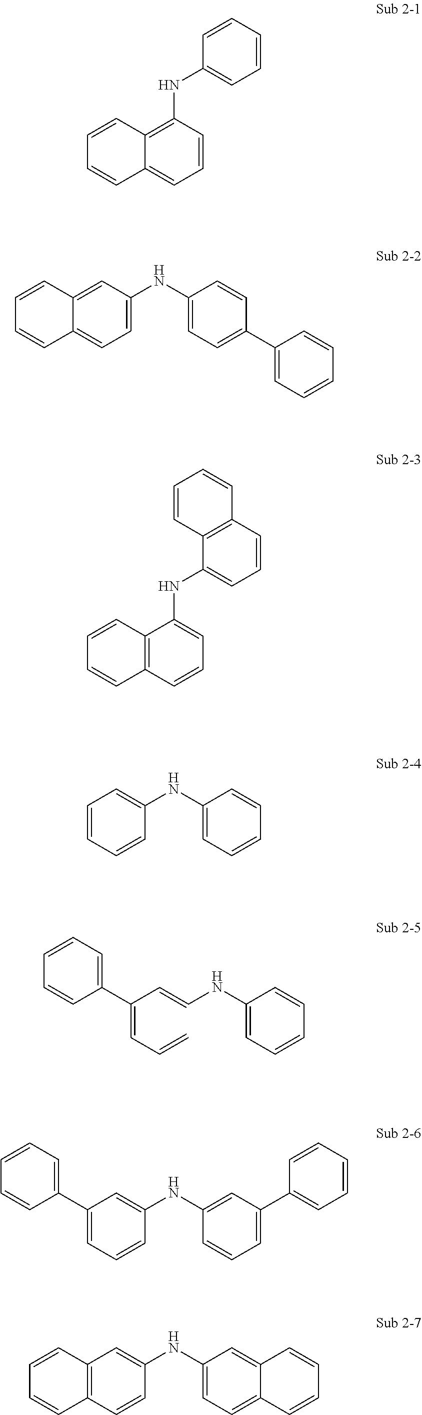

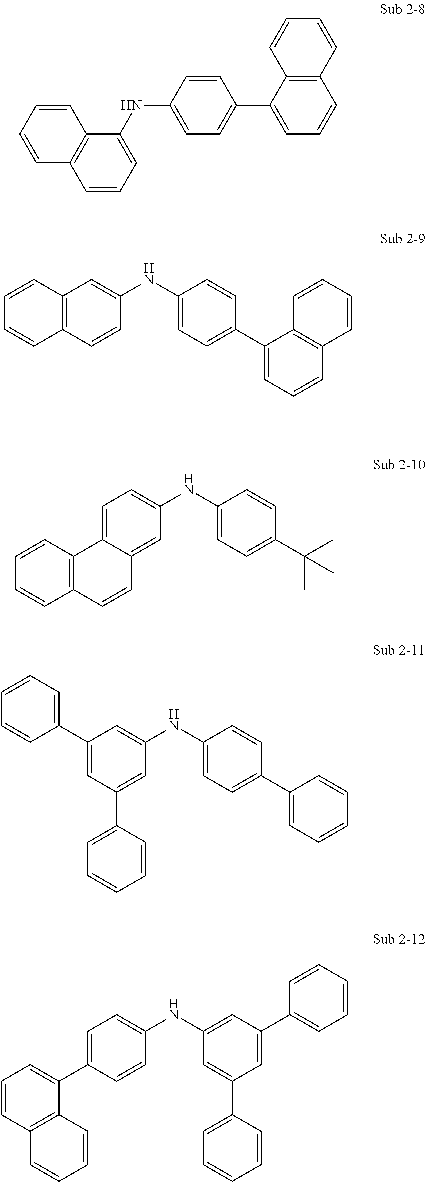 [SMILES: c1ccc2c(Nc3ccc(-c4cccc5ccccc45)cc3)cccc2c1 ; c1ccc2cc(Nc3ccc(-c4cccc5ccccc45)cc3)ccc2c1 ; CC(C)(C)c1ccc(Nc2ccc3c(ccc4ccccc43)c2)cc1 ; c1ccc(-c2ccc(Nc3cc(-c4ccccc4)cc(-c4ccccc4)c3)cc2)cc1 ; c1ccc(-c2cc(Nc3ccc(-c4cccc5ccccc45)cc3)cc(-c3ccccc3)c2)cc1]

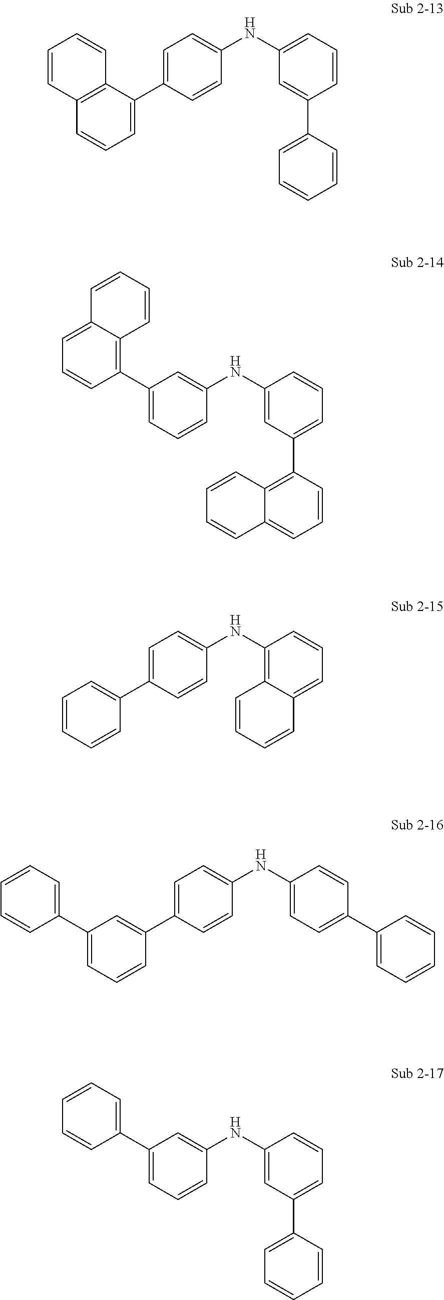 [SMILES: c1ccc(-c2cccc(Nc3ccc(-c4cccc5ccccc45)cc3)c2)cc1 ; c1cc(Nc2cccc(-c3cccc4ccccc34)c2)cc(-c2cccc3ccccc23)c1 ; c1ccc(-c2ccc(Nc3cccc4ccccc34)cc2)cc1 ; c1ccc(-c2ccc(Nc3ccc(-c4cccc(-c5ccccc5)c4)cc3)cc2)cc1 ; c1ccc(-c2cccc(Nc3cccc(-c4ccccc4)c3)c2)cc1]

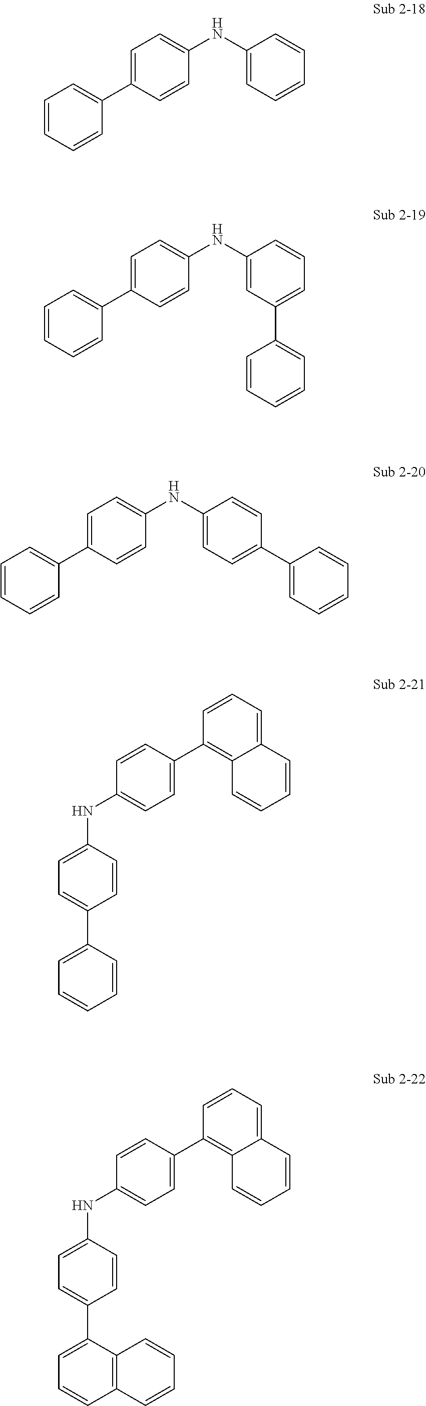 [SMILES: c1ccc(Nc2ccc(-c3ccccc3)cc2)cc1 ; c1ccc(-c2ccc(Nc3cccc(-c4ccccc4)c3)cc2)cc1 ; c1ccc(-c2ccc(Nc3ccc(-c4ccccc4)cc3)cc2)cc1 ; c1ccc(-c2ccc(Nc3ccc(-c4cccc5ccccc45)cc3)cc2)cc1 ; c1ccc2c(-c3ccc(Nc4ccc(-c5cccc6ccccc56)cc4)cc3)cccc2c1]

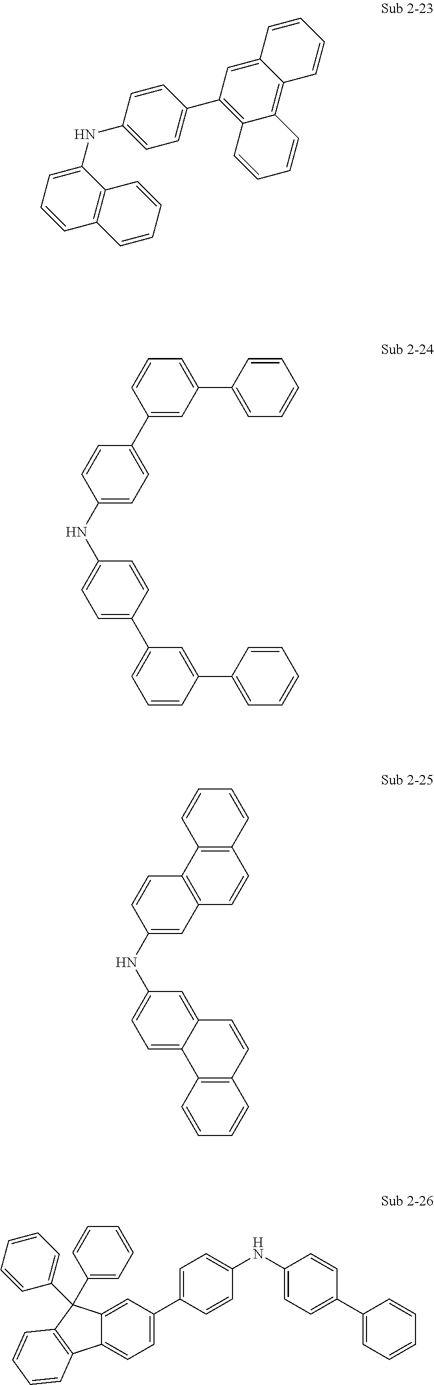 [SMILES: c1ccc2c(Nc3ccc(-c4cc5ccccc5c5ccccc45)cc3)cccc2c1 ; c1ccc(-c2cccc(-c3ccc(Nc4ccc(-c5cccc(-c6ccccc6)c5)cc4)cc3)c2)cc1 ; c1ccc2c(c1)ccc1cc(Nc3ccc4c(ccc5ccccc54)c3)ccc12 ; c1ccc(-c2ccc(Nc3ccc(-c4ccc5c(c4)C(c4ccccc4)(c4ccccc4)c4ccccc4-5)cc3)cc2)cc1]

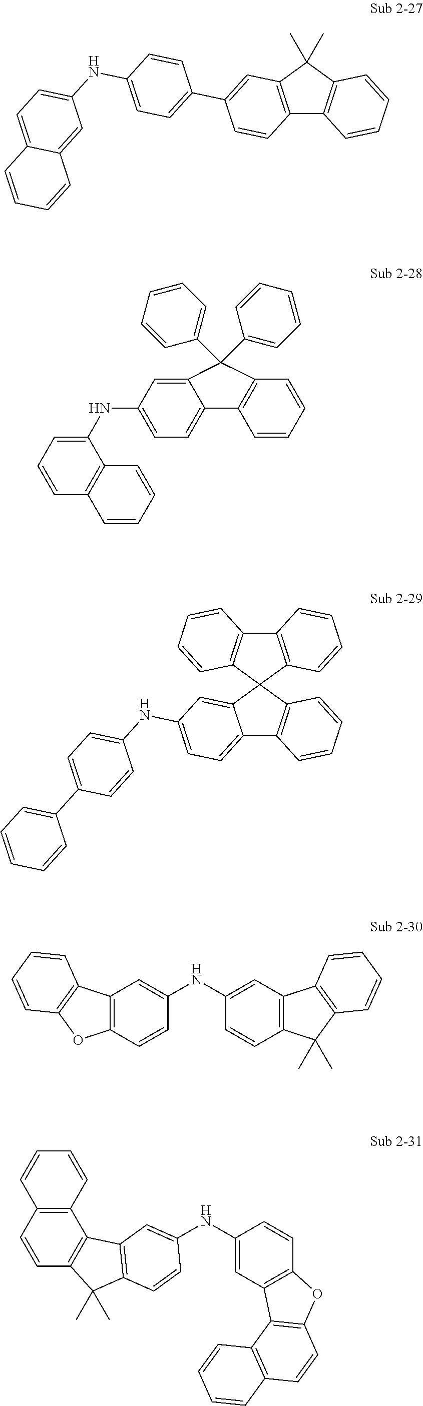 [SMILES: CC1(C)c2ccccc2-c2ccc(-c3ccc(Nc4ccc5ccccc5c4)cc3)cc21 ; c1ccc(C2(c3ccccc3)c3ccccc3-c3ccc(Nc4cccc5ccccc45)cc32)cc1 ; c1ccc(-c2ccc(Nc3ccc4c(c3)C3(c5ccccc5-c5ccccc53)c3ccccc3-4)cc2)cc1 ; CC1(C)c2ccccc2-c2cc(Nc3ccc4oc5ccccc5c4c3)ccc21 ; CC1(C)c2ccc(Nc3ccc4oc5ccc6ccccc6c5c4c3)cc2-c2c1ccc1ccccc21]

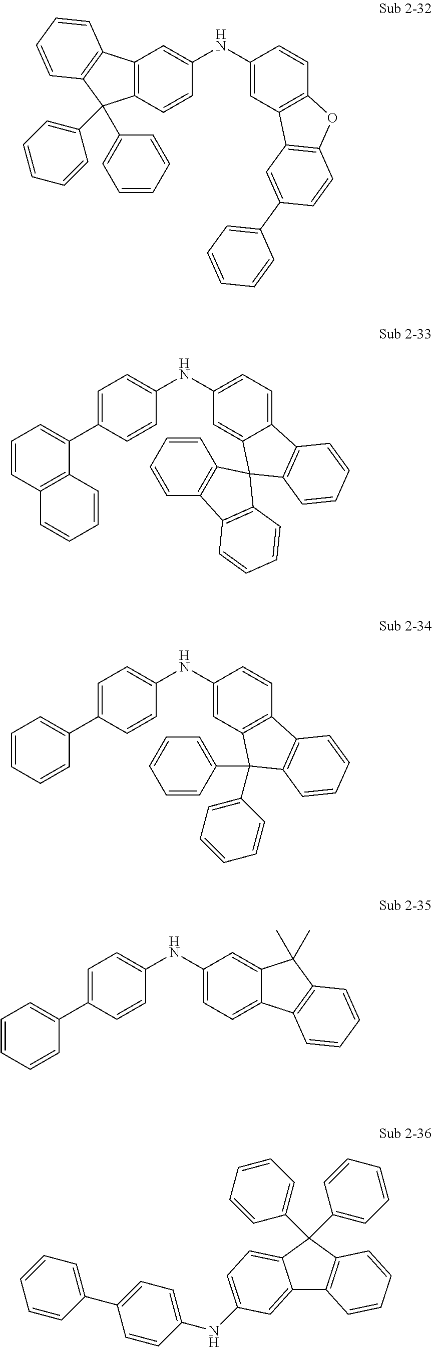 [SMILES: c1ccc(-c2ccc3oc4ccc(Nc5ccc6c(c5)-c5ccccc5C6(c5ccccc5)c5ccccc5)cc4c3c2)cc1 ; c1ccc2c(c1)-c1ccccc1C21c2ccccc2-c2ccc(Nc3ccc(-c4cccc5ccccc45)cc3)cc21 ; c1ccc(-c2ccc(Nc3ccc4c(c3)C(c3ccccc3)(c3ccccc3)c3ccccc3-4)cc2)cc1 ; CC1(C)c2ccccc2-c2ccc(Nc3ccc(-c4ccccc4)cc3)cc21 ; c1ccc(-c2ccc(Nc3ccc4c(c3)-c3ccccc3C4(c3ccccc3)c3ccccc3)cc2)cc1]

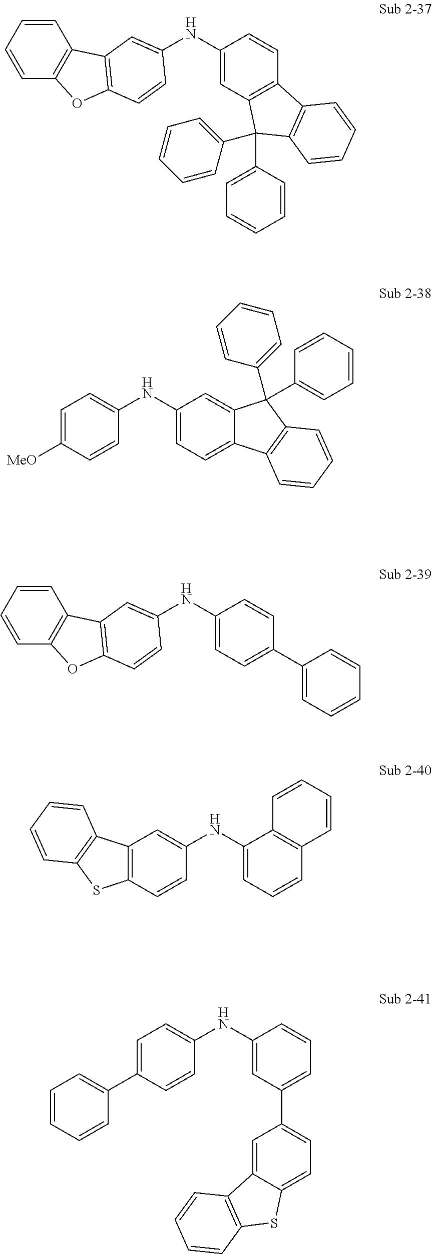 [SMILES: c1ccc(C2(c3ccccc3)c3ccccc3-c3ccc(Nc4ccc5oc6ccccc6c5c4)cc32)cc1 ; COc1ccc(Nc2ccc3c(c2)C(c2ccccc2)(c2ccccc2)c2ccccc2-3)cc1 ; c1ccc(-c2ccc(Nc3ccc4oc5ccccc5c4c3)cc2)cc1 ; c1ccc2c(Nc3ccc4sc5ccccc5c4c3)cccc2c1 ; c1ccc(-c2ccc(Nc3cccc(-c4ccc5sc6ccccc6c5c4)c3)cc2)cc1]

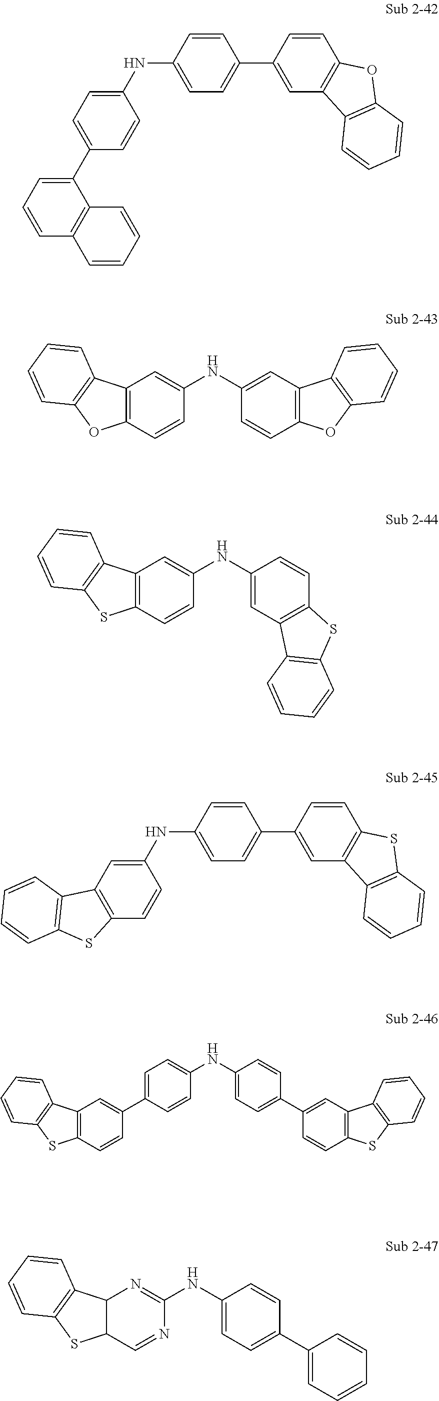 [SMILES: c1ccc2c(-c3ccc(Nc4ccc(-c5ccc6oc7ccccc7c6c5)cc4)cc3)cccc2c1 ; c1ccc2c(c1)oc1ccc(Nc3ccc4oc5ccccc5c4c3)cc12 ; c1ccc2c(c1)sc1ccc(Nc3ccc4sc5ccccc5c4c3)cc12 ; c1ccc2c(c1)sc1ccc(Nc3ccc(-c4ccc5sc6ccccc6c5c4)cc3)cc12 ; c1ccc2c(c1)sc1ccc(-c3ccc(Nc4ccc(-c5ccc6sc7ccccc7c6c5)cc4)cc3)cc12 ; C1=NC(Nc2ccc(-c3ccccc3)cc2)=NC2c3ccccc3SC12]

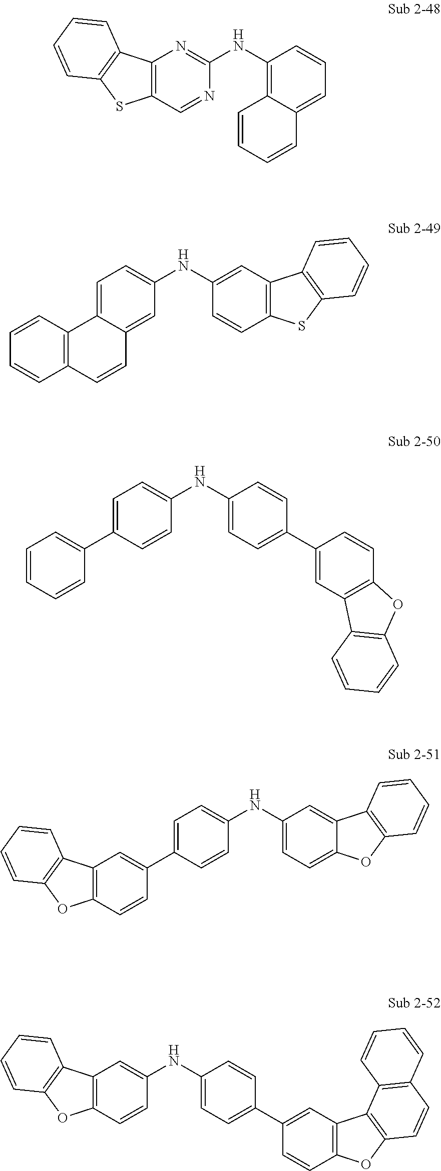 [SMILES: c1ccc2c(Nc3ncc4sc5ccccc5c4n3)cccc2c1 ; c1ccc2c(c1)ccc1cc(Nc3ccc4sc5ccccc5c4c3)ccc12 ; c1ccc(-c2ccc(Nc3ccc(-c4ccc5oc6ccccc6c5c4)cc3)cc2)cc1 ; c1ccc2c(c1)oc1ccc(Nc3ccc(-c4ccc5oc6ccccc6c5c4)cc3)cc12 ; c1ccc2c(c1)ccc1oc3ccc(-c4ccc(Nc5ccc6oc7ccccc7c6c5)cc4)cc3c12]

TABLE 1

| compound | FD-MS | compound | FD-MS |
|---|---|---|---|
| Sub 2-1 | m/z = 219.10(C₁₆H₁₃N = 219.28) | Sub 2-2 | m/z = 295.14(C₂₂H₁₇N = 295.38) |
| Sub 2-3 | m/z = 269.12(C₂₀H₁₅N = 269.34) | Sub 2-4 | m/z = 169.09(C₁₂H₁₁N = 169.22) |
| Sub 2-5 | m/z = 245.12(C₁₈H₁₅N = 245.32) | Sub 2-6 | m/z = 321.15(C₂₄H₁₉N = 321.41) |
| Sub 2-7 | m/z = 269.12(C₂₀H₁₅N = 269.34) | Sub 2-8 | m/z = 345.15(C₂₆H₁₉N = 345.44) |
| Sub 2-9 | m/z = 345.15(C₂₆H₁₉N = 345.44) | Sub 2-10 | m/z = 325.18(C₂₄H₂₃N = 325.45) |
| Sub 2-11 | m/z = 397.18(C₃₀H₂₃N = 397.51) | Sub 2-12 | m/z = 447.20(C₃₄H₂₅N = 447.57) |
| Sub 2-13 | m/z = 371.17(C₂₈H₂₁N = 371.47) | Sub 2-14 | m/z = 421.18(C₃₂H₂₃N = 421.53) |
| Sub 2-15 | m/z = 295.14(C₂₂H₁₇N = 295.38) | Sub 2-16 | m/z = 397.18(C₃₀H₂₃N = 397.51) |
| Sub 2-17 | m/z = 321.15(C₂₄H₁₉N = 321.41) | Sub 2-18 | m/z = 245.12(C₁₈H₁₅N = 245.32) |
| Sub 2-19 | m/z = 321.15(C₂₄H₁₉N = 321.41) | Sub 2-20 | m/z = 321.15(C₂₄H₁₉N = 321.41) |
| Sub 2-21 | m/z = 371.17(C₂₈H₂₁N = 371.47) | Sub 2-22 | m/z = 421.18(C₃₂H₂₃N = 421.53) |
| Sub 2-23 | m/z = 395.17(C₃₀H₂₁N = 395.49) | Sub 2-24 | m/z = 473.21(C₃₆H₂₇N = 473.61) |
| Sub 2-25 | m/z = 369.15(C₂₈H₁₉N = 369.46) | Sub 2-26 | m/z = 561.25(C₄₃H₃₁N = 561.71) |
| Sub 2-27 | m/z = 411.20(C₃₁H₂₅N = 411.54) | Sub 2-28 | m/z = 459.20(C₃₅H₂₅N = 459.58) |
| Sub 2-29 | m/z = 483.20(C₃₇H₂₅N = 483.60) | Sub 2-30 | m/z = 375.16(C₂₇H₂₁NO = 375.46) |
| Sub 2-31 | m/z = 475.19(C₃₅H₂₅NO = 475.58) | Sub 2-32 | m/z = 575.22(C₄₃H₂₉NO = 575.70) |
| Sub 2-33 | m/z = 533.21(C₄₁H₂₇N = 533.66) | Sub 2-34 | m/z = 485.21(C₃₇H₂₇N = 485.62) |
| Sub 2-35 | m/z = 361.18(C₂₇H₂₃N = 361.48) | Sub 2-36 | m/z = 485.21(C₃₇H₂₇N = 485.62) |
| Sub 2-37 | m/z = 499.19(C₃₇H₂₅NO = 499.60) | Sub 2-38 | m/z = 439.19(C₃₂H₂₅NO = 439.55) |
| Sub 2-39 | m/z = 335.13(C₂₄H₁₇NO = 335.40) | Sub 2-40 | m/z = 325.09(C₂₂H₁₅NS = 325.43) |
| Sub 2-41 | m/z = 427.14(C₃₀H₂₁NS = 427.56) | Sub 2-42 | m/z = 461.18(C₃₄H₂₃NO = 461.55) |
| Sub 2-43 | m/z = 349.11(C₂₄H₁₅NO₂ = 349.38) | Sub 2-44 | m/z = 381.06(C₂₄H₁₅NS₂ = 381.51) |
| Sub 2-45 | m/z = 457.10(C₃₀H₁₉NS₂ = 457.61) | Sub 2-46 | m/z = 533.13(C₃₆H₂₃NS₂ = 533.70) |
| Sub 2-47 | m/z = 353.10(C₂₂H₁₅N₃S = 353.44) | Sub 2-48 | m/z = 327.0(C₂₀H₁₃N₃S = 327.40) |
| Sub 2-49 | m/z = 375.11(C₂₆H₁₇NS = 375.48) | Sub 2-50 | m/z = 411.16(C₃₀H₂₁NO = 411.49) |
| Sub 2-51 | m/z = 425.14(C₃₀H₁₉NO₂ = 425.48) | Sub 2-52 | m/z = 475.16(C₃₄H₂₁NO₂ = 475.54) |

### Synthesis of Final Product of the Formula 1 (The Same Procedure of Sub 2)

Sub 2 (1 eq.) and Sub 1 (1.1 eq.) was dissolved in Toluene, Pd₂(dba)₃ (0.05 eq.), PPh₃(0.1 eq.), NaOt-Bu(3 eq.) were added in a round bottom flask, followed by stirring and reflux at 100° C. When the reaction is complete, the product was extracted with ether and water. The organic layer was dried over MgSO₄ and concentrated, and then the product was separated by a silicagel column chromatography and recrystallized to obtain the Final Product.

### Synthesis Example of Final Product

### Synthesis 1-1′

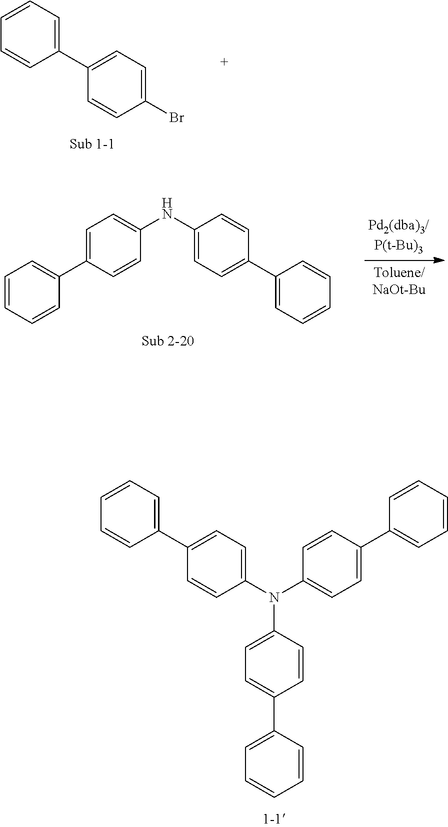 [SMILES: Brc1ccc(-c2ccccc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccccc4)cc3)cc2)cc1]

After di([1,1′-biphenyl]-4-yl)amine (10 g, 31.1 mmol), 4-bromo-1,1′-biphenyl(8 g, 34.2 mmol), Pd₂(dba)₃ (1.42 g, 1.56 mmol), P(t-Bu)₃ (0.63 g, 3.11 mmol), NaOt-Bu (136.9 g, 102.7 mmol), toluene (330 mL) are added in a round bottom flask, stirring at 100° C. When the reaction is complete, the product was extracted with CH₂Cl₂ and water. The organic layer was dried over MgSO₄ and concentrated, and then the product was separated by a silicagel column chromatography and recrystallized to obtain 11.3 g of Product 1-1′ (yield: 77%).

Synthesis 1-4′

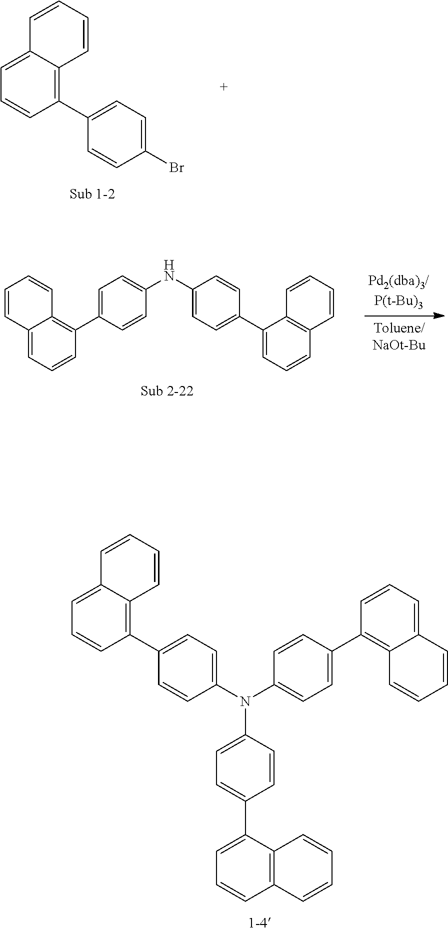 [SMILES: Brc1ccc(-c2cccc3ccccc23)cc1 ; c1ccc2c(-c3ccc(Nc4ccc(-c5cccc6ccccc56)cc4)cc3)cccc2c1 ; c1ccc2c(-c3ccc(N(c4ccc(-c5cccc6ccccc56)cc4)c4ccc(-c5cccc6ccccc56)cc4)cc3)cccc2c1]

Bis(4-(naphthalen-1-yl)phenyl)amine (10 g, 23.7 mmol), 1-(4-bromophenyl)naphthalene (7.4 g, 26.1 mmol), Pd₂(dba)₃ (1.09 g, 1.19 mmol), P(t-Bu)₃ (0.5 g, 2.4 mmol), NaOt-Bu (104 g, 78.3 mmol), toluene (250 mL) were added in a round bottom flask, and the same procedure as described in the synthesis method of Product 1-1′ was carried out to obtain 11.5 g of Product 1-4′ (yield: 78%).

### Synthesis 1-10′

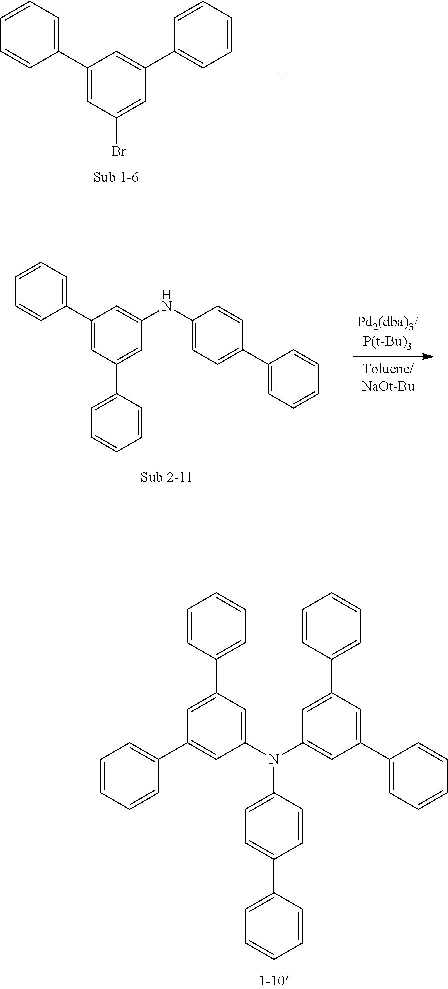 [SMILES: Brc1cc(-c2ccccc2)cc(-c2ccccc2)c1 ; c1ccc(-c2ccc(Nc3cc(-c4ccccc4)cc(-c4ccccc4)c3)cc2)cc1 ; c1ccc(-c2ccc(N(c3cc(-c4ccccc4)cc(-c4ccccc4)c3)c3cc(-c4ccccc4)cc(-c4ccccc4)c3)cc2)cc1]

N-([1,1′-biphenyl]-4-yl)-[1,1′:3′,1″-terphenyl]-5′-amine (10 g, 25.2 mmol), 5′-bromo-1,1′:3′,1″-terphenyl (8.56 g, 27.7 mmol), Pd₂(dba)₃ (1.15 g, 1.26 mmol), P(t-Bu)₃ (0.51 g, 2.52 mmol), NaOt-Bu (110 g, 54-83.02 mmol), toluene (264 mL) were added in a round bottom flask, and the same procedure as described in the synthesis method of Product 1-1′ was carried out to obtain 11.8 g of Product 1-10′ (yield: 75%).

### Synthesis 1-19′

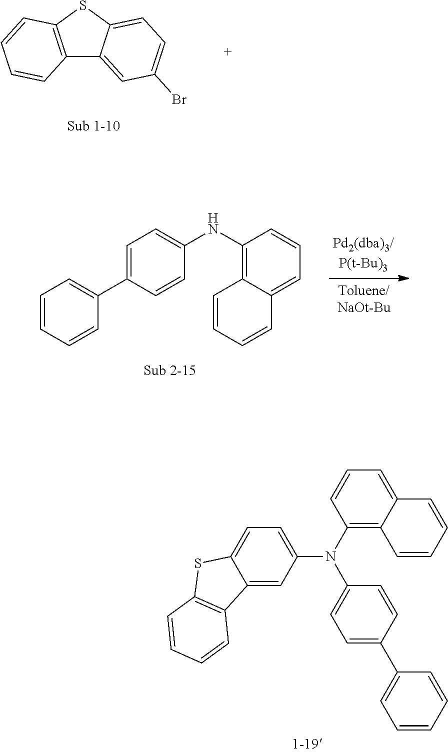 [SMILES: Brc1ccc2sc3ccccc3c2c1 ; c1ccc(-c2ccc(Nc3cccc4ccccc34)cc2)cc1 ; c1ccc(-c2ccc(N(c3ccc4sc5ccccc5c4c3)c3cccc4ccccc34)cc2)cc1]

N-([1,1′-biphenyl]-4-yl)naphthalen-1-amine (10 g, 33.6 mmol), 2-bromodibenzo[b,d]thiophene (9.8 g, 37.2 mmol), Pd₂(dba)₃ (1.55 g, 1.7 mmol), P(t-Bu)₃ (0.68 g, 3.38 mmol), NaOt-Bu (149 g, 112 mmol), toluene (355 mL) were added in a round bottom flask, and the same procedure as described in the synthesis method of Product 1-1′ was carried out to obtain 12.3 g of Product 1-19′ (yield: 76%).

### Synthesis 1-20′

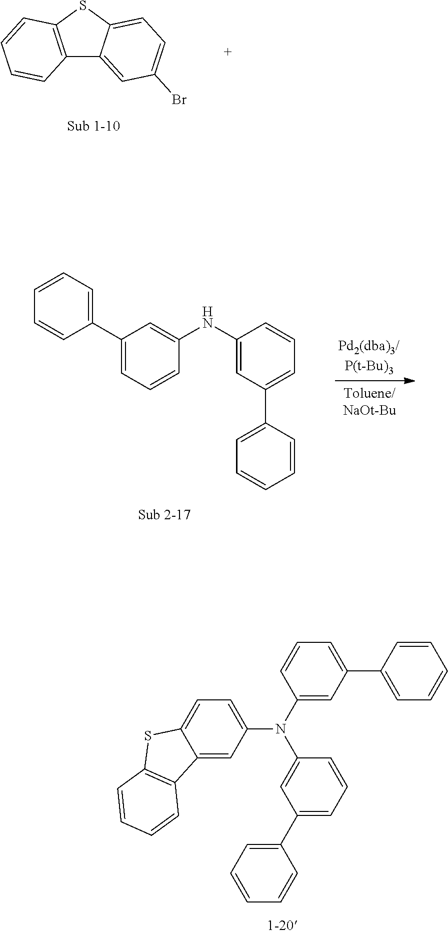 [SMILES: Brc1ccc2sc3ccccc3c2c1 ; c1ccc(-c2cccc(Nc3cccc(-c4ccccc4)c3)c2)cc1 ; c1ccc(-c2cccc(N(c3cccc(-c4ccccc4)c3)c3ccc4sc5ccccc5c4c3)c2)cc1]

Di([1,1′-biphenyl]-3-yl)amine (10 g, 31.1 mmol), 2-bromodibenzo[b,d]thiophene (9 g, 34.2 mmol), Pd₂(dba)₃ (1.42 g, 1.56 mmol), P(t-Bu)₃ (0.63 g, 3.11 mmol), NaOt-Bu (136.9 g, 102.7 mmol), toluene (327 mL) were added in a round bottom flask, and the same procedure as described in the synthesis method of Product 1-1′ was carried out to obtain 12.2 g of Product 1-20′ (yield: 78%).

### Synthesis 1-23′

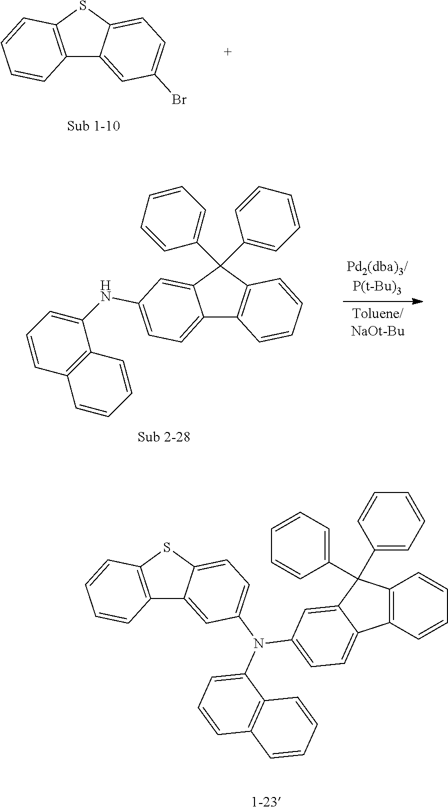 [SMILES: Brc1ccc2sc3ccccc3c2c1 ; c1ccc(C2(c3ccccc3)c3ccccc3-c3ccc(Nc4cccc5ccccc45)cc32)cc1 ; c1ccc(C2(c3ccccc3)c3ccccc3-c3ccc(N(c4ccc5sc6ccccc6c5c4)c4cccc5ccccc45)cc32)cc1]

N-(naphthalen-1-yl)-9,9-diphenyl-9H-fluoren-2-amine (10 g, 21.8 mmol), 2-bromodibenzo[b,d]thiophene (6.3 g, 23.9 mmol), Pd₂(dba)₃ (1 g, 1.09 mmol), P(t-Bu)₃ (0.44 g, 2.2 mmol), NaOt-Bu (95.7 g, 71.8 mmol), toluene (230 mL) were added in a round bottom flask, and the same procedure as described in the synthesis method of Product 1-1′ was carried out to obtain 10.2 g of Product 1-23′ (yield: 73%).

### Synthesis 1-24′

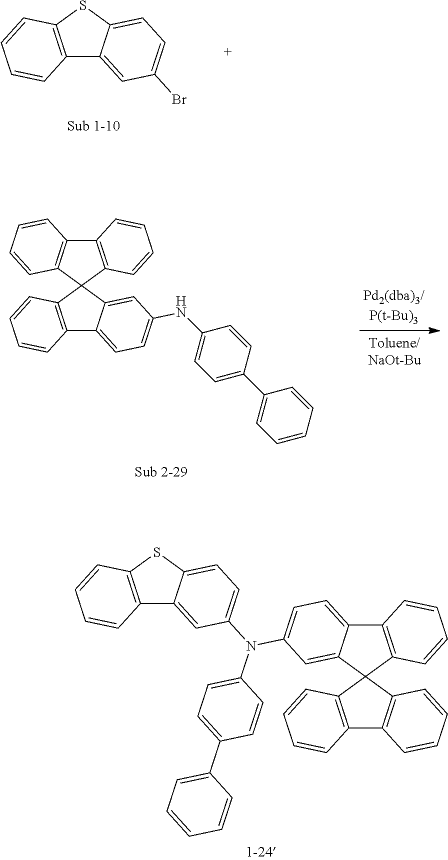 [SMILES: Brc1ccc2sc3ccccc3c2c1 ; c1ccc(-c2ccc(Nc3ccc4c(c3)C3(c5ccccc5-c5ccccc53)c3ccccc3-4)cc2)cc1 ; c1ccc(-c2ccc(N(c3ccc4c(c3)C3(c5ccccc5-c5ccccc53)c3ccccc3-4)c3ccc4sc5ccccc5c4c3)cc2)cc1]

N-([1,1′-biphenyl]-4-yl)-9,9′-spirobi[fluoren]-2-amine (10 g, 20.7 mmol), 2-bromodibenzo[b,d]thiophene (6 g, 22.7 mmol), Pd₂(dba)₃ (0.95 g, 1.03 mmol), P(t-Bu)₃ (0.42 g, 2.07 mmol), NaOt-Bu (91 g, 68.2 mmol), and toluene (220 mL) were added in a round bottom flask, and the same procedure as described in the synthesis method of Product 1-1′ was carried out to obtain 10.2 g of Product 1-24′ (yield: 74%).

### Synthesis 1-29′

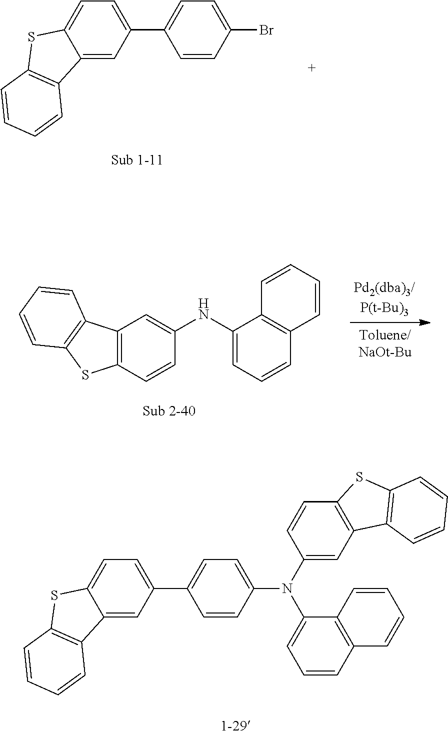 [SMILES: Brc1ccc(-c2ccc3sc4ccccc4c3c2)cc1 ; c1ccc2c(Nc3ccc4sc5ccccc5c4c3)cccc2c1 ; c1ccc2c(N(c3ccc(-c4ccc5sc6ccccc6c5c4)cc3)c3ccc4sc5ccccc5c4c3)cccc2c1]

N-(naphthalen-1-yl)dibenzo[b,d]thiophen-2-amine (10 g, 30.7 mmol), 2-(4-bromophenyl)dibenzo[b,d]thiophene (11.5 g, 33.8 mmol), Pd₂(dba)₃ (1.41 g, 1.54 mmol), P(t-Bu)₃ (0.62 g, 3.07 mmol), NaOt-Bu (135.2 g, 101.4 mmol) and toluene (325 mL) were added in a round bottom flask, and the same procedure as described in the synthesis method of Product 1-1′ was carried out to obtain 12.9 g of Product 1-29′ (yield: 72%).

### Synthesis 1-30′

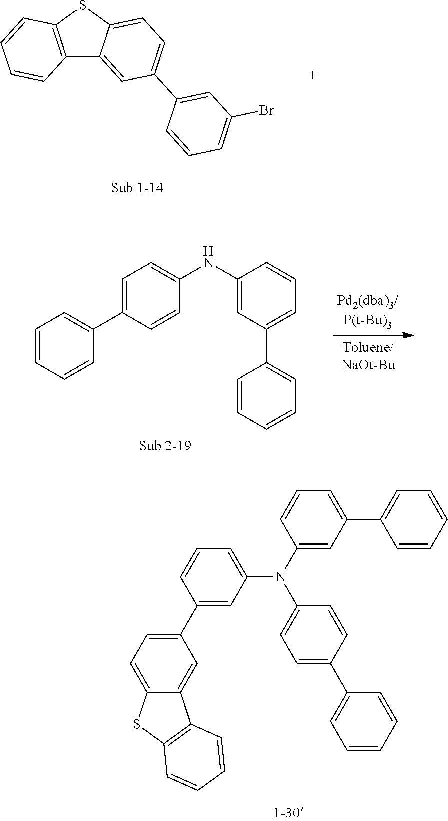 [SMILES: Brc1cccc(-c2ccc3sc4ccccc4c3c2)c1 ; c1ccc(-c2ccc(Nc3cccc(-c4ccccc4)c3)cc2)cc1 ; c1ccc(-c2ccc(N(c3cccc(-c4ccccc4)c3)c3cccc(-c4ccc5sc6ccccc6c5c4)c3)cc2)cc1]

N-([1,1′-biphenyl]-4-yl)-[1,1′-biphenyl]-3-amine (10 g, 31.1 mmol), 2-(3-bromophenyl)dibenzo[b,d]thiophene (11.6 g, 34.2 mmol), Pd₂(dba)₃ (1.42 g, 1.55 mmol), P(t-Bu)₃ (0.63 g, 3.11 mmol), NaOt-Bu (137 g, 103 mmol) and toluene (330 mL) were added in a round bottom flask, and the same procedure as described in the synthesis method of Product 1-1′ was carried out to obtain 12.8 g of Product 1-30′ (yield: 71%).

### Synthesis 1-36′

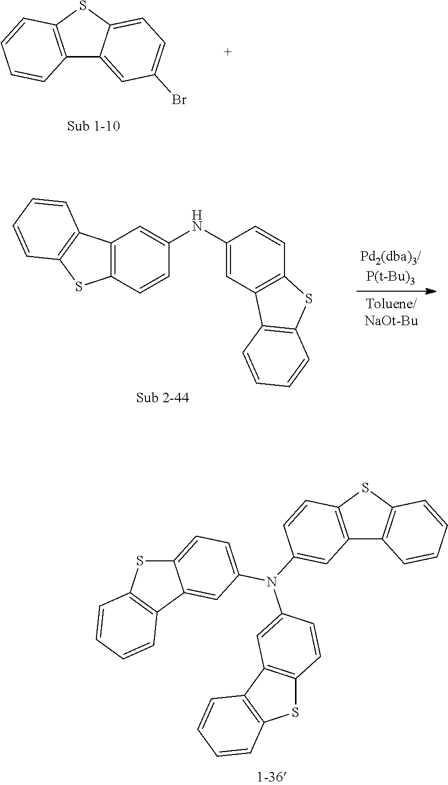 [SMILES: Brc1ccc2sc3ccccc3c2c1 ; c1ccc2c(c1)sc1ccc(Nc3ccc4sc5ccccc5c4c3)cc12 ; c1ccc2c(c1)sc1ccc(N(c3ccc4sc5ccccc5c4c3)c3ccc4sc5ccccc5c4c3)cc12]

Bis(dibenzo[b,d]thiophen-2-yl)amine (10 g, 26.2 mmol), 2-bromodibenzo[b,d]thiophene (7.59 g, 28.8 mmol), Pd₂(dba)₃ (1.2 g, 1.31 mmol), P(t-Bu)₃ (0.53 g, 2.62 mmol), NaOt-Bu (115.3 g, 86.5 mmol) and toluene (275 mL) were added in a round bottom flask, and the same procedure as described in the synthesis method of Product 1-1′ was carried out to obtain 11.4 g of Product 1-36′ (yield: 77%).

### Synthesis 1-49′

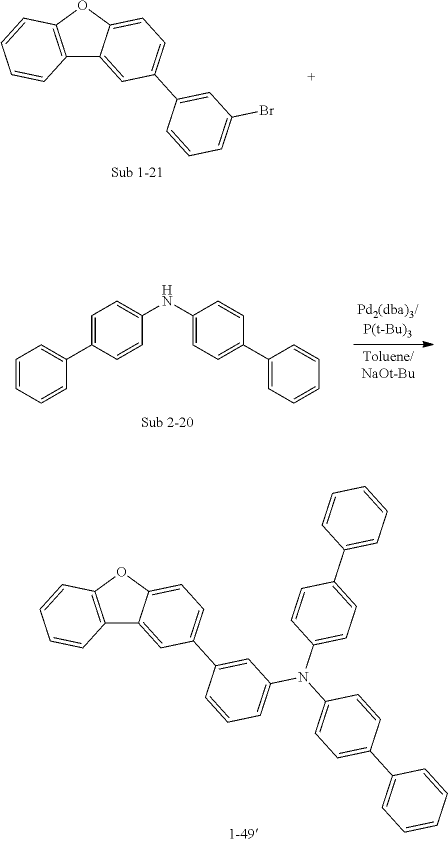 [SMILES: Brc1cccc(-c2ccc3oc4ccccc4c3c2)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccc5oc6ccccc6c5c4)c3)cc2)cc1]

Di([1,1′-biphenyl]-4-yl)amine (10 g, 31.1 mmol), 2-(3-bromophenyl)dibenzo[b,d]furan (11.1 g, 34.2 mmol), Pd₂(dba)₃ (1.42 g, 1.56 mmol), P(t-Bu)₃ (0.63 g, 3.11 mmol), NaOt-Bu (137 g, 103 mmol), toluene (330 mL) were added in a round bottom flask, and the same procedure as described in the synthesis method of Product 1-1′ was carried out to obtain 13.3 g of Product 1-49′ (yield: 76%).

### Synthesis 1-51′

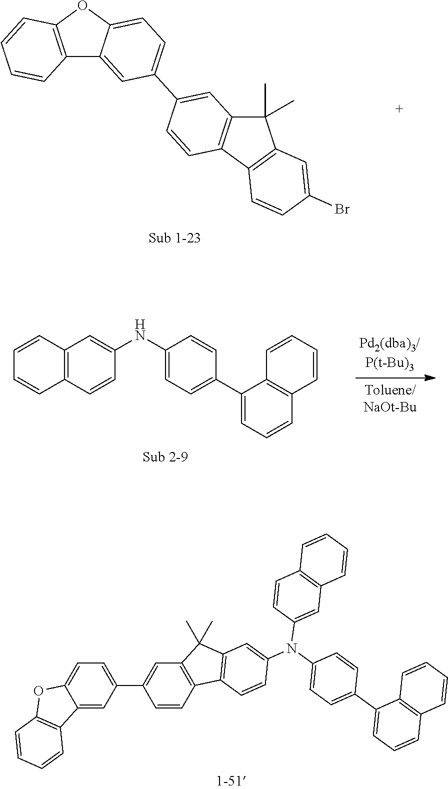 [SMILES: CC1(C)c2cc(Br)ccc2-c2ccc(-c3ccc4oc5ccccc5c4c3)cc21 ; c1ccc2cc(Nc3ccc(-c4cccc5ccccc45)cc3)ccc2c1 ; CC1(C)c2cc(-c3ccc4oc5ccccc5c4c3)ccc2-c2ccc(N(c3ccc(-c4cccc5ccccc45)cc3)c3ccc4ccccc4c3)cc21]

N-(4-(naphthalen-1-yl)phenyl)naphthalen-2-amine (10 g, 28.9 mmol), 2-(7-bromo-9,9-dimethyl-9H-fluoren-2-yl)dibenzo[b,d]furan (14 g, 32 mmol), Pd₂(dba)₃ (1.33 g, 1.45 mmol), P(t-Bu)₃ (0.59 g, 2.9 mmol), NaOt-Bu (127.4 g, 95.5 mmol) and toluene (310 mL) were added in a round bottom flask, and the same procedure as described in the synthesis method of Product 1-1′ was carried out to obtain 14.5 g of Product 1-51′ (yield: 71%).

### Synthesis 1-59′

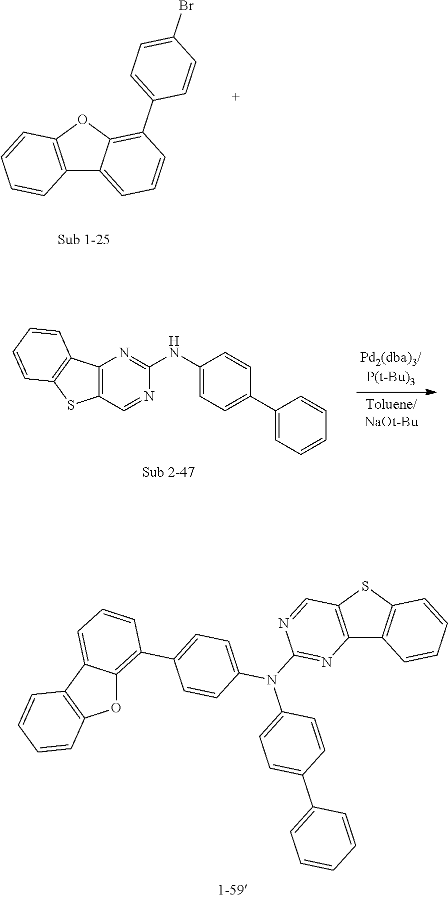 [SMILES: Brc1ccc(-c2cccc3c2oc2ccccc23)cc1 ; c1ccc(-c2ccc(Nc3ncc4sc5ccccc5c4n3)cc2)cc1 ; c1ccc(-c2ccc(N(c3ccc(-c4cccc5c4oc4ccccc45)cc3)c3ncc4sc5ccccc5c4n3)cc2)cc1]

N-([1,1′-biphenyl]-4-yl)benzo[4,5]thieno[3,2-d]pyrimidin-2-amine (10 g, 28.3 mmol), 4-(4-bromophenyl)dibenzo[b,d]furan (10.1 g, 31.1 mmol), Pd₂(dba)₃ (1.3 g, 1.41 mmol), P(t-Bu)₃ (0.57 g, 2.83 mmol), NaOt-Bu (124.5 g, 93.4 mmol) and toluene (300 mL) were added in a round bottom flask, and the same procedure as described in the synthesis method of Product 1-1′ above was carried out to obtain 12.3 g of Product 1-59′ (yield: 73%).

### Synthesis 1-71′

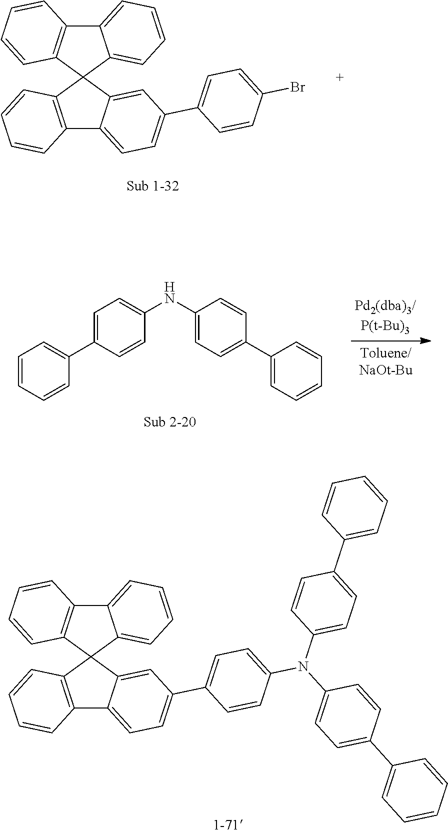 [SMILES: Brc1ccc(-c2ccc3c(c2)C2(c4ccccc4-c4ccccc42)c2ccccc2-3)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc5c(c4)C4(c6ccccc6-c6ccccc64)c4ccccc4-5)cc3)cc2)cc1]

Di([1,1′-biphenyl]-4-yl)amine (10 g, 31.1 mmol), 2-(4-bromophenyl)-9,9′-spirobi[fluorene] (16.1 g, 34.2 mmol), Pd₂(dba)₃ (1.42 g, 1.56 mmol), P(t-Bu)₃ (0.63 g, 3.11 mmol), NaOt-Bu (136.9 g, 102.7 mmol), and toluene (330 mL) were added in a round bottom flask, and the same procedure as described in the synthesis method of Product 1-1′ above was carried out to obtain 15.5 g of Product 1-71′ (yield: 70%).

### Synthesis 1-75′

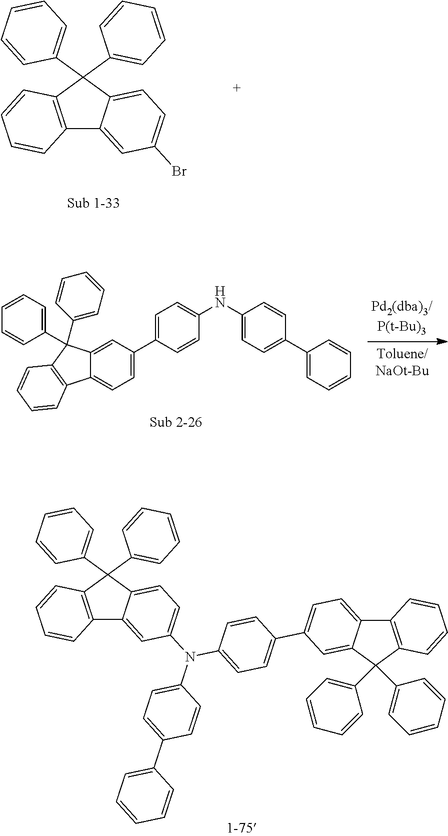 [SMILES: Brc1ccc2c(c1)-c1ccccc1C2(c1ccccc1)c1ccccc1 ; c1ccc(-c2ccc(Nc3ccc(-c4ccc5c(c4)C(c4ccccc4)(c4ccccc4)c4ccccc4-5)cc3)cc2)cc1 ; c1ccc(-c2ccc(N(c3ccc(-c4ccc5c(c4)C(c4ccccc4)(c4ccccc4)c4ccccc4-5)cc3)c3ccc4c(c3)-c3ccccc3C4(c3ccccc3)c3ccccc3)cc2)cc1]

N-(4-(9,9-diphenyl-9H-fluoren-2-yl)phenyl)-[1,1′-biphenyl]-4-amine (10 g, 17.8 mmol), 3-bromo-9,9-diphenyl-9H-fluorene (7.78 g, 19.6 mmol), Pd₂(dba)₃ (0.82 g, 0.89 mmol), P(t-Bu)₃ (0.36 g, 1.78 mmol), NaOt-Bu (78.3 g, 58.75 mmol), and toluene (190 mL) were added in a round bottom flask, and the same procedure as described in the synthesis method of Product 1-1′ above was carried out to obtain 11.3 g of Product 1-75′ (yield: 72%).

TABLE 2

| compound | FD-MS | compound | FD-MS |
|---|---|---|---|
| 1-1′ | m/z = 473.21(C₃₆H₂₇N = 473.61) | 1-2′ | m/z = 523.23(C₄₀H₂₉N = 523.66) |
| 1-3′ | m/z = 573.25(C₄₄H₃₁N = 573.72) | 1-4′ | m/z = 623.26(C₄₈H₃₃N = 623.78) |
| 1-5′ | m/z = 447.20(C₃₄H₂₅N = 447.57) | 1-6′ | m/z = 371.17(C₂₈H₂₁N = 371.47) |
| 1-7′ | m/z = 471.20(C₃₆H₂₅N = 471.59) | 1-8′ | m/z = 521.21(C₄₀H₂₇N = 521.65) |
| 1-9′ | m/z = 549.25(C₄₂H₃₁N = 549.70) | 1-10′ | m/z = 625.28(C₄₈H₃₅N = 625.80) |
| 1-11′ | m/z = 675.29(C₅₂H₃₇N = 675.86) | 1-12′ | m/z = 473.21(C₃₆H₂₇N = 473.61) |
| 1-13′ | m/z = 523.23(C₄₀H₂₉N = 523.66) | 1-14′ | m/z = 623.26(C₄₈H₃₃N = 623.78) |
| 1-15′ | m/z = 549.25(C₄₂H₃₁N = 549.70) | 1-16′ | m/z = 625.28(C₄₈H₃₅N = 625.80) |
| 1-17′ | m/z = 503.17(C₃₆H₂₅NS = 503.66) | 1-18′ | m/z = 603.20(C₄₄H₂₉NS = 603.77) |
| 1-19′ | m/z = 477.16(C₃₄H₂₃NS = 477.62) | 1-20′ | m/z = 503.17(C₃₆H₂₅NS = 503.66) |
| 1-21′ | m/z = 451.14(C₃₂H₂₁NS = 451.58) | 1-22′ | m/z = 593.22(C₄₃H₃₁NS = 593.78) |
| 1-23′ | m/z = 641.22(C₄₇H₃₁NS = 641.82) | 1-24′ | m/z = 665.22(C₄₉H₃₁NS = 665.84) |
| 1-25′ | m/z = 503.17(C₃₆H₂₅NS = 503.66) | 1-26′ | m/z = 655.23(C₄₈H₃₃NS = 655.85) |
| 1-27′ | m/z = 695.26(C₅₁H₃₇NS = 695.91) | 1-28′ | m/z = 593.18(C₄₂H₂₇NOS = 593.73) |
| 1-29′ | m/z = 583.14(C₄₀H₂₅NS₂ = 583.76) | 1-30′ | m/z = 579.20(C₄₂H₂₉NS = 579.75) |
| 1-31′ | m/z = 685.19(C₄₈H₃₁NS₂ = 685.90) | 1-32′ | m/z = 719.23(C₅₂H₃₃NOS = 719.89) |
| 1-33′ | m/z = 629.22(C₄₆H₃₁NS = 629.81) | 1-34′ | m/z = 629.22(C₄₆H₃₁NS = 629.81) |
| 1-35′ | m/z = 603.20(C₄₄H₂₉NS = 603.77) | 1-36′ | m/z = 563.08(C₃₆H₂₁NS₃ = 563.75) |
| 1-37′ | m/z = 639.11(C₄₂H₂₅NS₃ = 639.85) | 1-38′ | m/z = 715.15(C₄₈H₂₉NS₃ = 715.95) |
| 1-39′ | m/z = 791.18(C₅₄H₃₃NS₃ = 792.04) | 1-40′ | m/z = 607.16(C₄₂H₂₅NO₂S = 607.72) |
| 1-41′ | m/z = 633.21(C₄₅H₃₁NOS = 633.80) | 1-42′ | m/z = 733.24(C₅₃H₃₅NOS = 733.92) |
| 1-43′ | m/z = 883.29(C₆₅H₄₁NOS = 884.09) | 1-44′ | m/z = 585.13(C₃₈H₂₃N₃S₂ = 585.74) |
| 1-45′ | m/z = 553.19(C₄₀H₂₇NS = 553.71) | 1-46′ | m/z = 603.20(C₄₄H₂₉NS = 603.77) |
| 1-47′ | m/z = 841.28(C₆₃H₃₉NS = 842.06) | 1-48′ | m/z = 563.22(C₄₂H₂₉NO = 563.69) |
| 1-49′ | m/z = 563.22(C₄₂H₂₉NO = 563.69) | 1-50′ | m/z = 613.24(C₄₆H₃₁NO = 613.74) |
| 1-51′ | m/z = 703.29(C₅₃H₃₇NO = 703.87) | 1-52′ | m/z = 587.22(C₄₄H₂₉NO = 587.71) |
| 1-53′ | m/z = 563.22(C₄₂H₂₉NO = 563.69) | 1-54′ | m/z = 639.26(C₄₈H₃₃NO = 639.78) |
| 1-55′ | m/z = 653.24(C₄₈H₃₁NO₂ = 653.77) | 1-56′ | m/z = 603.26(C₄₅H₃₃NO = 603.75) |
| 1-57′ | m/z = 727.29(C₅₅H₃₇NO = 727.89) | 1-58′ | m/z = 725.27(C₅₅H₃₅NO = 725.87) |
| 1-59′ | m/z = 595.17(C₄₀H₂₅N₃OS = 595.71) | 1-60′ | m/z = 567.26(C₄₂H₃₃NO = 567.72) |
| 1-61′ | m/z = 611.22(C₄₆H₂₉NO = 611.73) | 1-62′ | m/z = 617.18(C₄₄H₂₇NOS = 617.76) |
| 1-63′ | m/z = 637.24(C₄₈H₃₁NO = 637.77) | 1-64′ | m/z = 667.21(C₄₈H₂₉NO₃ = 667.75) |
| 1-65′ | m/z = 767.25(C₅₆H₃₃NO₃ = 767.87) | 1-66′ | m/z = 681.27(C₅₀H₃₅NO₂ = 681.82) |
| 1-67′ | m/z = 713.31(C₅₅H₃₉N = 713.90) | 1-68′ | m/z = 589.28(C₄₅H₃₅N = 589.77) |
| 1-69′ | m/z = 639.29(C₄₉H₃₇N = 639.82) | 1-70′ | m/z = 613.28(C₄₇H₃₅N = 613.79) |
| 1-71′ | m/z = 711.29(C₅₆H₃₇N = 711.89) | 1-72′ | m/z = 637.28(C₄₉H₃₅N = 637.81) |
| 1-73′ | m/z = 761.31(C₅₉H₃₉N = 761.95) | 1-74′ | m/z = 637.28(C₄₉H₃₅N = 637.81) |
| 1-75′ | m/z = 877.37(C₆₈H₄₇N = 878.11) | 1-76′ | m/z = 875.36(C₆₈H₄₅N = 876.09) |
| 1-77′ | m/z = 813.30(C₆₂H₃₉NO = 813.98) | | |

The synthesis method is based on the Buchwald-Hartwing cross coupling reaction.

### Manufacture and Evaluation of Organic Electric Element

### [Example I-1] Blue Organic Light Emitting Diode (Hole Transport Layer)

Using the compound of the present invention as a hole transport layer material, an organic electric element was manufactured according to a conventional method. First, on an ITO layer(anode) formed on a glass substrate, 4,4′,4″-Tris[2-naphthyl(phenyl)amino]triphenylamine (hereinafter will be abbreviated as 2-TNATA) was vacuum-deposited to form a hole injection layer with a thickness of 60 nm, and on the hole transport layer, the mixture of the present invention was vacuum deposited to form a hole transport layer with a thickness of 60 nm. Then, on the hole transport layer, an emitting layer with a thickness of 30 nm was deposited using 9,10-di(naphthalen-2-yl)anthracene, as a host doped with BD-052X (Idemitsukosan) as a dopant in a weight ratio of 95:5. (1,1′-bisphenyl)-4-olato)bis(2-methyl-8-quinolinolato)aluminum (hereinafter will be abbreviated as BAlq) was vacuum deposited to form a hole blocking layer with a thickness of 10 nm, and an electron transport layer was formed by vacuum-depositing tris (8-quinolinol) aluminum (hereinafter will be abbreviated as Alq3) to a thickness of 40 nm. After that, an alkali metal halide, LiF was vacuum deposited as an electron injection layer to a thickness of 0.2 nm, and Al was deposited to a thickness of 150 nm to form a cathod to manufacture an OLED.

### Comparative Example 1

Except for using the following comparative compound 1 to comparative compound 4 for the hole transport layer material instead of using the mixture of the present invention, an OLED was manufactured in the same manner as described in the example I-1.

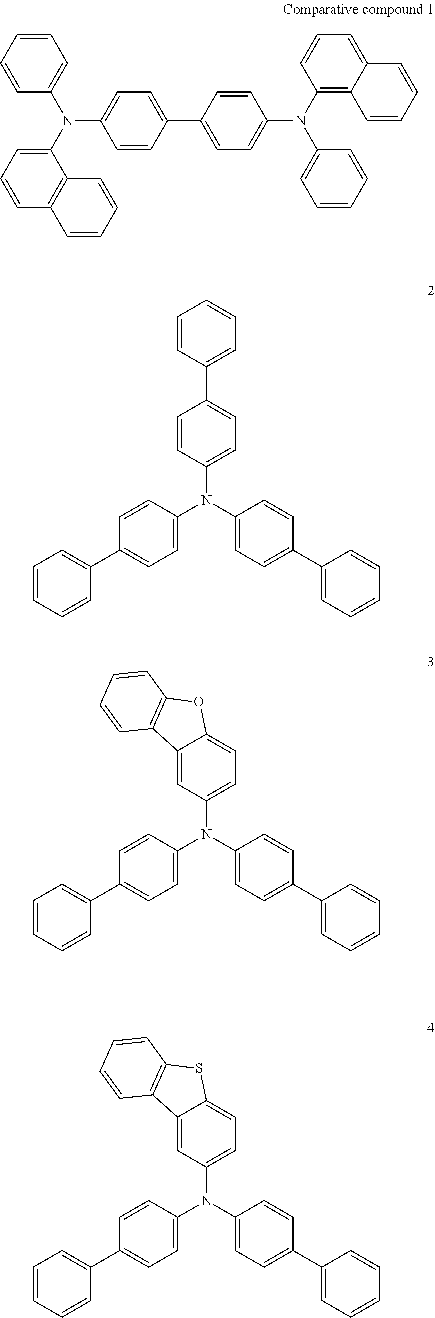 [SMILES: c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc4oc5ccccc5c4c3)cc2)cc1 ; c1ccc(-c2ccc(N(c3ccc(-c4ccccc4)cc3)c3ccc4sc5ccccc5c4c3)cc2)cc1]

To the OLEDs which were manufactured by examples and comparative example 1 to comparative example 4, a forward bias direct current voltage was applied, and electroluminescent (EL) properties were measured using PR-650 of Photoresearch Co., and T95 life was measured using a life measuring apparatus manufactured by McScience Inc. with a reference luminance of 500 cd/m². In the following table, the manufacture of advice and the results of evaluation are shown.

TABLE 3

| | | | | | Electric | | | | |
|---|---|---|---|---|---|---|---|---|---|
| | Mixing | | | Driving | current | luminance | efficiency | Luminous | |
| | ratio | Compound A | compound B | voltage | (mA/cm²) | (cd/cm²) | (cd/A) | color | T(95) |
| comparative | single | comparative | None | 4.5 | 15.8 | 500 | 3.2 | blue | 83 |
| example (1) | compound | compound (1) | | | | | | | |
| comparative | single | comparative | None | 4.5 | 12.3 | 500 | 4.1 | blue | 92 |
| example (2) | compound | compound (2) | | | | | | | |
| comparative | single | comparative | None | 4.9 | 9.6 | 500 | 5.2 | blue | 97 |
| example (3) | compound | compound (3) | | | | | | | |
| comparative | single | comparative | None | 4.9 | 10.0 | 500 | 5.0 | blue | 94 |
| example (4) | compound | compound (4) | | | | | | | |
| example (1) | A(2):B(8) | compound | compound | 4.4 | 9.1 | 500 | 5.0 | blue | 103.7 |
| | | 1-1′ | 1-3′ | | | | | | |
| example (2) | A(2):B(8) | compound | compound | 4.5 | 8.8 | 500 | 5.2 | blue | 108.3 |
| | | 1-1′ | 1-4′ | | | | | | |
| example (3) | A(2):B(8) | Compound | compound | 4.5 | 9.1 | 500 | 5.0 | blue | 103.2 |
| | | 1-1′ | 1-9′ | | | | | | |
| example (4) | A(2):B(8) | Compound | compound | 4.6 | 9.0 | 500 | 5.0 | blue | 106.8 |
| | | 1-1′ | 1-10′ | | | | | | |
| example (5) | A(2):B(8) | compound | compound | 4.6 | 9.2 | 500 | 4..9 | blue | 107.0 |
| | | 1-1′ | 1-12′ | | | | | | |
| example (6) | A(2):B(8) | compound | compound | 4.4 | 9.1 | 500 | 4.8 | blue | 102.4 |
| | | 1-1′ | 1-14′ | | | | | | |
| example (7) | A(2):B(8) | Compound | compound | 4.5 | 8.5 | 500 | 5.9 | blue | 107.6 |
| | | 1-1′ | 1-17′ | | | | | | |
| example (8) | A(2):B(8) | Compound | compound | 4.6 | 8.6 | 500 | 5.8 | blue | 106.1 |
| | | 1-1′ | 1-19′ | | | | | | |
| example (9) | A(2):B(8) | Compound | compound | 4.6 | 8.6 | 500 | 5.8 | blue | 108.2 |
| | | 1-1′ | 1-25′ | | | | | | |
| example (10) | A(2):B(8) | compound | compound | 4.5 | 8.6 | 500 | 5.8 | blue | 106.2 |
| | | 1-1′ | 1-26′ | | | | | | |
| example (11) | A(2):B(8) | Compound | compound | 4.5 | 7.9 | 500 | 6.3 | blue | 107.9 |
| | | 1-1′ | 1-48′ | | | | | | |
| example (12) | A(2):B(8) | compound | compound | 4.6 | 7.6 | 500 | 6.6 | blue | 108.0 |
| | | 1-1′ | 1-52′ | | | | | | |
| example (13) | A(2):B(8) | compound | compound | 4.5 | 7.8 | 500 | 6.4 | blue | 107.2 |
| | | 1-1′ | 1-57′ | | | | | | |
| example (14) | A(2):B(8) | Compound | compound | 4.7 | 8.8 | 500 | 5.7 | blue | 104.7 |
| | | 1-1′ | 1-72′ | | | | | | |
| example (15) | A(3):B(7) | compound | compound | 4.6 | 8.2 | 500 | 6.1 | blue | 116.5 |
| | | 1-1′ | 1-4′ | | | | | | |
| example (16) | A(3):B(7) | Compound | compound | 4.5 | 7.8 | 500 | 6.4 | blue | 117.3 |
| | | 1-1′ | 1-17′ | | | | | | |
| example (17) | A(3):B(7) | Compound | compound | 4.4 | 8.1 | 500 | 6.2 | blue | 117.6 |
| | | 1-1′ | 1-26′ | | | | | | |
| example (18) | A(3):B(7) | Compound | compound | 4.5 | 7.5 | 500 | 6.7 | blue | 123.3 |
| | | 1-1′ | 1-48′ | | | | | | |
| example (19) | A(3):B(7) | compound | compound | 4.5 | 7.5 | 500 | 6.7 | blue | 122.0 |
| | | 1-1′ | 1-52′ | | | | | | |
| example (20) | A(3):B(7) | compound 1- | compound | 4.7 | 8.2 | 500 | 6.1 | blue | 115.2 |
| | | 1′ | 1-72′ | | | | | | |
| example (21) | A(4):B(6) | compound | compound | 4.6 | 8.2 | 500 | 6.1 | blue | 117.6 |
| | | 1-1′ | 1-4′ | | | | | | |
| example (22) | A(4):B(6) | Compound | compound | 4.5 | 7.6 | 500 | 6.6 | blue | 118.9 |
| | | 1-1′ | 1-17′ | | | | | | |
| example (23) | A(4):B(6) | compound | compound | 4.5 | 7.6 | 500 | 6.6 | blue | 117.2 |
| | | 1-1′ | 1-26′ | | | | | | |
| example (24) | A(4):B(6) | Compound | compound | 4.4 | 7.2 | 500 | 6.9 | blue | 125.6 |
| | | 1-1′ | 1-48′ | | | | | | |
| example (25) | A(4):B(6) | Compound | compound | 4.6 | 7.2 | 500 | 6.9 | blue | 125.1 |
| | | 1-1′ | 1-52′ | | | | | | |
| example (26) | A(4):B(6) | Compound | compound | 4.7 | 8.1 | 500 | 6.2 | blue | 117.2 |
| | | 1-1′ | 1-72′ | | | | | | |
| example (27) | A(5):B(5) | Compound | compound | 4.6 | 7.9 | 500 | 6.3 | blue | 123.6 |
| | | 1-1′ | 1-4′ | | | | | | |
| example (28) | A(5):B(5) | compound | compound | 4.5 | 7.5 | 500 | 6.7 | blue | 126.6 |
| | | 1-1′ | 1-17′ | | | | | | |
| example (29) | A(5):B(5) | Compound | compound | 4.6 | 7.2 | 500 | 6.9 | blue | 127.4 |
| | | 1-1′ | 1-26′ | | | | | | |
| example (30) | A(5):B(5) | Compound | compound | 4.6 | 6.9 | 500 | 7.2 | blue | 130.6 |
| | | 1-1′ | 1-48′ | | | | | | |
| example (31) | A(5):B(5) | Compound | compound | 4.6 | 6.8 | 500 | 7.4 | blue | 134.0 |
| | | 1-1′ | 1-52′ | | | | | | |
| example (32) | A(5):B(5) | Compound | compound | 4.6 | 7.8 | 500 | 6.4 | blue | 122.3 |
| | | 1-1′ | 1-72′ | | | | | | |
| example (33) | A(7):B(3) | Compound | compound | 4.7 | 8.6 | 500 | 5.8 | blue | 105.9 |
| | | 1-1′ | 1-4′ | | | | | | |
| example (34) | A(7):B(3) | Compound | compound | 4.7 | 8.1 | 500 | 6.2 | blue | 108.1 |
| | | 1-1′ | 1-17′ | | | | | | |
| example (35) | A(7):B(3) | Compound | compound | 4.5 | 8.3 | 500 | 6.0 | blue | 107.2 |
| | | 1-1′ | 1-26′ | | | | | | |
| example (36) | A(7):B(3) | compound | compound | 4.6 | 8.2 | 500 | 6.1 | blue | 104.7 |
| | | 1-1′ | 1-48′ | | | | | | |
| example (37) | A(7):B(3) | Compound | compound | 4.6 | 8.3 | 500 | 6.0 | blue | 106.6 |
| | | 1-1′ | 1-52′ | | | | | | |
| example (38) | A(7):B(3) | Compound | compound | 4.5 | 8.5 | 500 | 5.9 | blue | 101.3 |
| | | 1-1′ | 1-72′ | | | | | | |
| example (39) | A(5):B(5) | Compound | compound | 4.4 | 7.0 | 500 | 7.1 | blue | 127.2 |
| | | 1-52′ | 1-17′ | | | | | | |
| example (40) | A(5):B(5) | Compound | compound | 4.7 | 6.8 | 500 | 7.4 | blue | 127.6 |
| | | 1-52′ | 1-18′ | | | | | | |
| example (41) | A(5):B(5) | Compound | compound | 4.5 | 7.1 | 500 | 7.0 | blue | 125.5 |
| | | 1-52′ | 1-22′ | | | | | | |
| example (42) | A(5):B(5) | Compound | compound | 4.5 | 7.0 | 500 | 7.1 | blue | 126.9 |
| | | 1-52′ | 1-24′ | | | | | | |
| example (43) | A(5):B(5) | Compound | compound | 4.6 | 6.8 | 500 | 7.4 | blue | 137.9 |
| | | 1-52′ | 1-33′ | | | | | | |
| example (44) | A(5):B(5) | Compound | compound | 4.4 | 7.1 | 500 | 7.0 | blue | 122.2 |
| | | 1-52′ | 1-34′ | | | | | | |
| example (45) | A(5):B(5) | Compound | compound | 4.6 | 6.9 | 500 | 7.2 | blue | 127.0 |
| | | 1-52′ | 1-52′ | | | | | | |
| example (46) | A(5):B(5) | Compound | compound | 4.7 | 7.0 | 500 | 7.1 | blue | 124.0 |
| | | 1-52′ | 1-57′ | | | | | | |
| example (47) | A(5):B(5) | Compound | compound | 4.7 | 7.1 | 500 | 7.0 | blue | 125.9 |
| | | 1-52′ | 1-58′ | | | | | | |
| example (48) | A(5):B(5) | Compound | compound | 4.6 | 6.9 | 500 | 7.2 | blue | 124.4 |
| | | 1-52′ | 1-67′ | | | | | | |
| example (49) | A(5):B(5) | Compound | compound | 4.5 | 7.0 | 500 | 7.1 | blue | 122.3 |
| | | 1-52′ | 1-74′ | | | | | | |
| example (50) | A(5):B(5) | Compound | compound | 4.6 | 7.0 | 500 | 7.1 | blue | 111.9 |
| | | 1-52′ | 1-28′ | | | | | | |
| example (51) | A(5):B(5) | Compound | compound | 4.5 | 7.0 | 500 | 7.1 | blue | 113.1 |
| | | 1-52′ | 1-29′ | | | | | | |
| example (52) | A(5):B(5) | Compound | compound | 4.4 | 7.1 | 500 | 7.0 | blue | 113.6 |
| | | 1-52′ | 1-31′ | | | | | | |
| example (53) | A(5):B(5) | Compound | compound | 4.6 | 7.0 | 500 | 7.1 | blue | 112.0 |
| | | 1-52′ | 1-32′ | | | | | | |
| example (54) | A(5):B(5) | Compound | compound | 4.6 | 7.0 | 500 | 7.1 | blue | 110.4 |
| | | 1-52′ | 1-36′ | | | | | | |
| example (55) | A(5):B(5) | Compound | compound | 4.6 | 7.0 | 500 | 7.1 | blue | 117.4 |
| | | 1-52′ | 1-39′ | | | | | | |
| example (56) | A(5):B(5) | Compound | compound | 4.6 | 7.0 | 500 | 7.2 | blue | 116.2 |
| | | 1-52′ | 1-44′ | | | | | | |
| example (57) | A(5):B(5) | Compound | compound | 4.7 | 7.1 | 500 | 7.0 | blue | 113.5 |
| | | 1-52′ | 1-55′ | | | | | | |
| example (58) | A(5):B(5) | compound | compound | 4.6 | 7.1 | 500 | 7.0 | blue | 112.5 |
| | | 1-52′ | 1-64′ | | | | | | |

As it is apparent from the results of Table 3, when the mixture of the present invention is used as hole transport layer, the luminous efficiency and life span can be remarkably improved as compared with the comparative example 1 to the comparative example 4 which are single compound.

The results of Table 3 will be described in more detail. First, example 1 to example 14 measured with the hole transport layer mixing a tertiary amine compound 1-1′ substituted with an aryl group biphenyl and a tertiary amine compound with other structure (comparative compound 1-3′, 1-4′, 1-9′, 1-10′, 1-12′, 1-14′, 1-17′, 1-19′, 1-25′, 1-26′, 1-48′, 1-52′, 1-57′, 1-72′) in the ratio of 2:8 (mixing ratio) increase efficiency and life span, and reduces the driving voltage in comparison with the comparative 1 to the comparative 4 using the hole transport layer having single compound.

Especially, when the mixture of the tertiary amine (1-3′, 1-4′, 1-9′, 1-10′, 1-12′, 1-14′) substituted with simple aryl and having different structures, and the compound 1-1′ is used as the hole transport layer, the efficiency increases 117%˜127%, and when the mixture of the tertiary amine (1-17′, 1-19′, 1-25′, 1-26′, 1-48′, 1-52′, 1-57′, 1-72′) comprising hetero ring compound and the compound 1-1′ is used as the hole transport layer, the efficiency increases 114%˜132%.

As a result of proceeding Examples 1 to 38 in order to investigate the differences in the characteristics of the mixing ratios, when the mixing ratio was 5:5, the highest efficiency was obtained and the life span was increased. In the case of Examples 33 to 38 in which the ratio of the compound 1-1 of all the amine substituents biphenyl is 7, and the ratio of the other tertiary amine is 3, efficiency and life span are reduced in comparison to the mixing ratio of 5:5.

Also for mixing materials, the mixture of the tertiary amine compound comprising the compound 1-1′ and the hetero ring group exhibited higher efficiency and longer life span than the mixture of the tertiary compound all substituted with the compound 1-1′ and the aryl group. Also, at the same mixing ratio, the mixtures containing compound 1-52′ containing Dibenzofuran exhibited higher efficiency and life span than the mixture of other compounds.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment.

The scope of the present invention shall be construed on the basis of the accompanying claims, and it shall be construed that all of the technical ideas included within the scope equivalent to the claims belong to the present invention.

What is claimed is:

1. An organic electric element comprising:
a first electrode,
a second electrode, and
an organic layer disposed between the first electrode and the second electrode and comprising a hole transport layer and an emitting layer comprising emitting compounds,
wherein the hole transport layer comprises a mixture of two compounds having different structures, one being represented by Formula 1-2, and the other being represented by Formula 1-3 or 1-4: 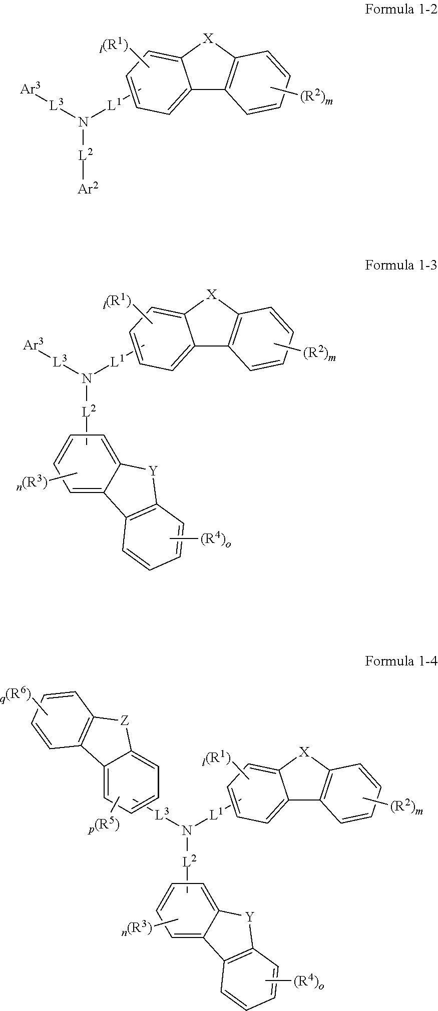
wherein:
1) Ar² and Ar³ are each independently selected from the group consisting of an unsubstituted C₆-C₆₀ aryl group, and a fluorenyl group, and L¹, L², L³ are each independently selected from the group consisting of a single bond, a C₆-C₆₀ arylene group, and a fluorenylene group,
2) X, Y and Z are each S, O, or CR′R″,
3) R′and R″ are selected from the group consisting of a C₆-C₂₄ aryl group, a C₁-C₂₀ alkyl group, and R′ and R″ may combine to each other and form a spiro ring,
4) R¹, R², R³, R⁴, R⁵, and R⁶ are each independently selected from the group consisting of deuterium, an C₆-C₆₀ aryl group, and an C₂-C₂₀ heterocyclic group, and a plurality of R¹ or a plurality of R² or a plurality of R³ or plurality of R⁴ or plurality of R⁵ or plurality of R⁶ may combine to each other to form a ring,
5) l, n, p are an integer of 0 to 3,
6) m, o, q are an integer of 0 to 4,
wherein the aryl group except for the unsubstituted C₆-C₆₀ aryl group as Ar² or Ar³, the fluorenyl or fluorenylene group, the arylene group, and the heterocyclic group may be substituted with one or more substituents selected from the group consisting of deuterium; halogen; a C₁-C₂₀ alkyl group; a C₆-C₆₀ aryl group; a C₆-C₆₀ aryl group substituted with deuterium; and a fluorenyl group.

2. The organic electric element according to claim 1, wherein the compound of Formula 1-2, 1-3 or 1-4 is represented by any of the following compounds:

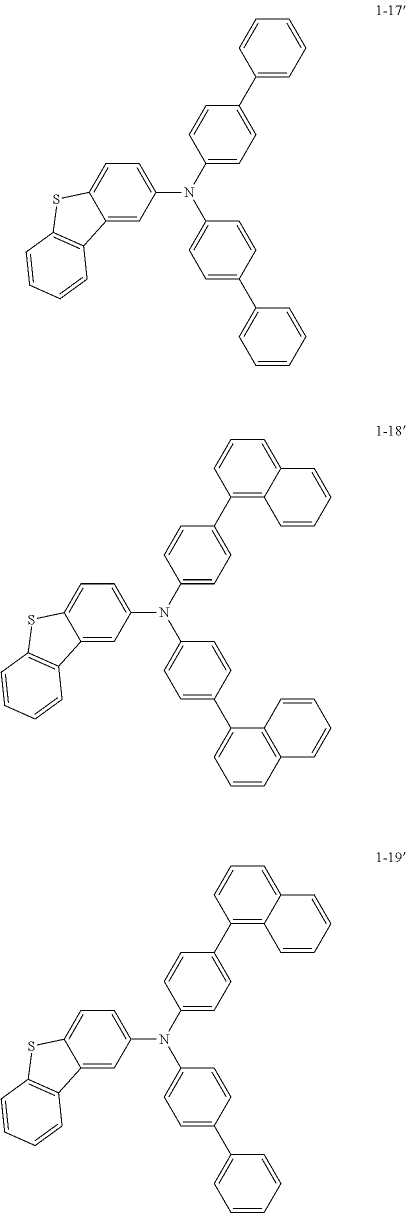 [SMILES: c1ccc(-c2ccc(N(c3ccc(-c4ccccc4)cc3)c3ccc4sc5ccccc5c4c3)cc2)cc1 ; c1ccc2c(-c3ccc(N(c4ccc(-c5cccc6ccccc56)cc4)c4ccc5sc6ccccc6c5c4)cc3)cccc2c1 ; c1ccc(-c2ccc(N(c3ccc(-c4cccc5ccccc45)cc3)c3ccc4sc5ccccc5c4c3)cc2)cc1]

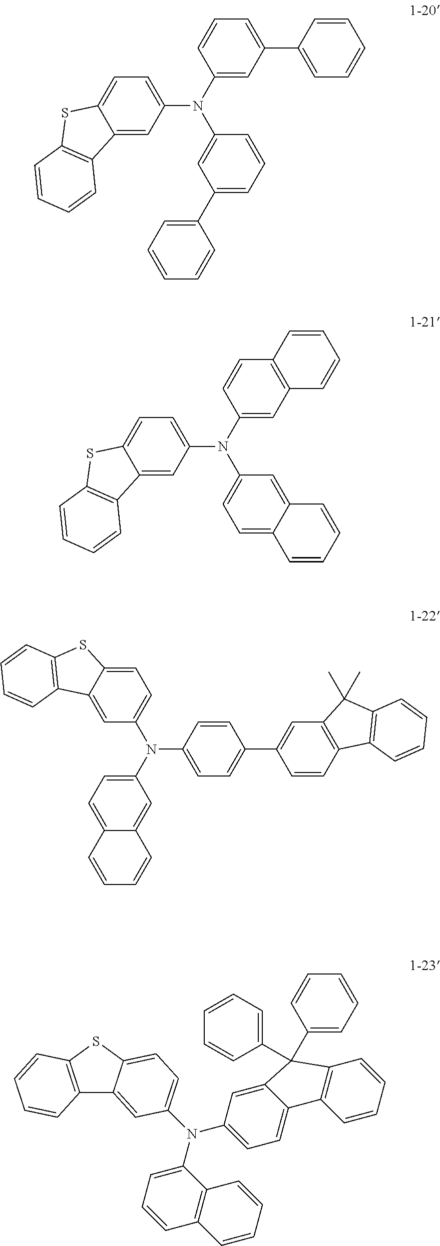 [SMILES: c1ccc(-c2cccc(N(c3cccc(-c4ccccc4)c3)c3ccc4sc5ccccc5c4c3)c2)cc1 ; c1ccc2cc(N(c3ccc4ccccc4c3)c3ccc4sc5ccccc5c4c3)ccc2c1 ; CC1(C)c2ccccc2-c2ccc(-c3ccc(N(c4ccc5ccccc5c4)c4ccc5sc6ccccc6c5c4)cc3)cc21 ; c1ccc(C2(c3ccccc3)c3ccccc3-c3ccc(N(c4ccc5sc6ccccc6c5c4)c4cccc5ccccc45)cc32)cc1]

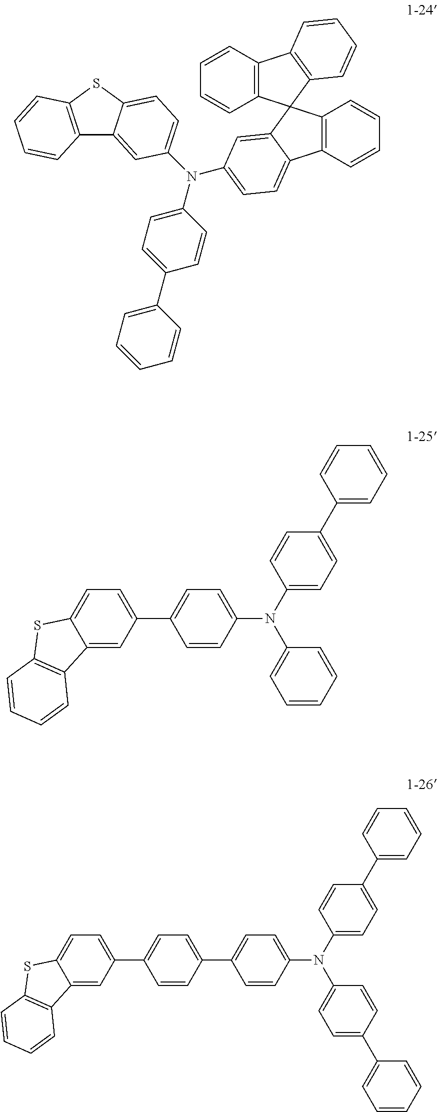 [SMILES: c1ccc(-c2ccc(N(c3ccc4c(c3)C3(c5ccccc5-c5ccccc53)c3ccccc3-4)c3ccc4sc5ccccc5c4c3)cc2)cc1 ; c1ccc(-c2ccc(N(c3ccccc3)c3ccc(-c4ccc5sc6ccccc6c5c4)cc3)cc2)cc1 ; c1ccc(-c2ccc(N(c3ccc(-c4ccccc4)cc3)c3ccc(-c4ccc(-c5ccc6sc7ccccc7c6c5)cc4)cc3)cc2)cc1]

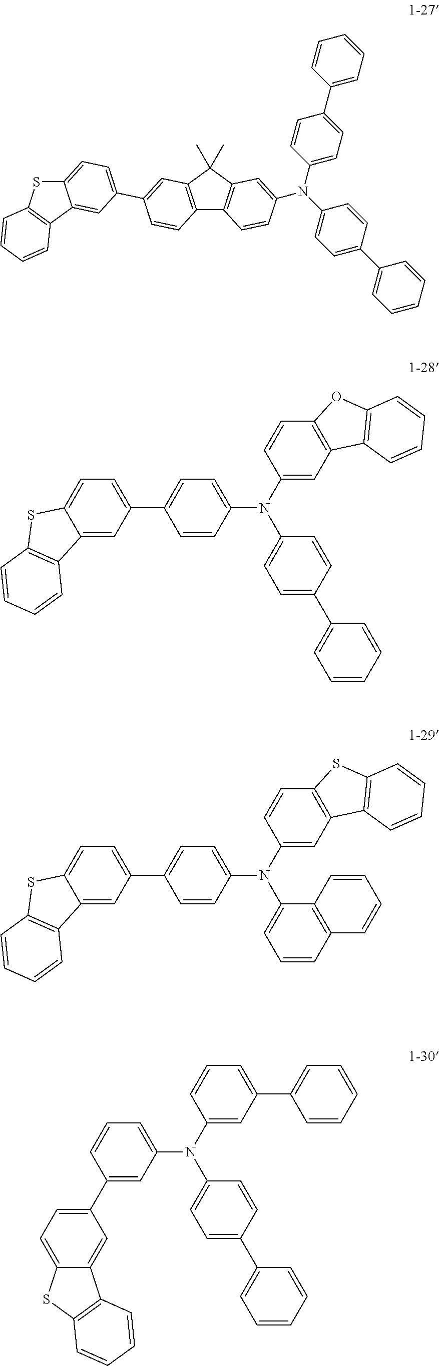 [SMILES: CC1(C)c2cc(-c3ccc4sc5ccccc5c4c3)ccc2-c2ccc(N(c3ccc(-c4ccccc4)cc3)c3ccc(-c4ccccc4)cc3)cc21 ; c1ccc(-c2ccc(N(c3ccc(-c4ccc5sc6ccccc6c5c4)cc3)c3ccc4oc5ccccc5c4c3)cc2)cc1 ; c1ccc2c(N(c3ccc(-c4ccc5sc6ccccc6c5c4)cc3)c3ccc4sc5ccccc5c4c3)cccc2c1 ; c1ccc(-c2ccc(N(c3cccc(-c4ccccc4)c3)c3cccc(-c4ccc5sc6ccccc6c5c4)c3)cc2)cc1]

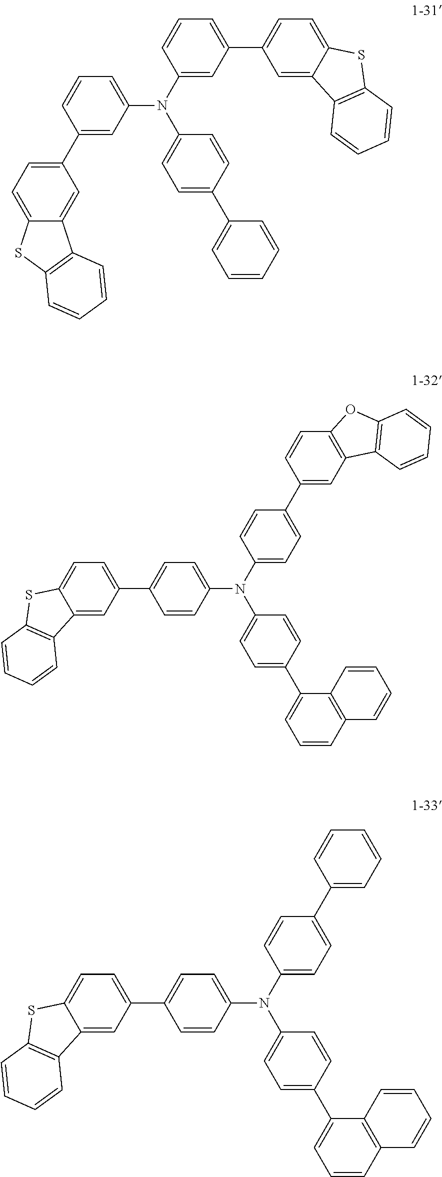 [SMILES: c1ccc(-c2ccc(N(c3cccc(-c4ccc5sc6ccccc6c5c4)c3)c3cccc(-c4ccc5sc6ccccc6c5c4)c3)cc2)cc1 ; c1ccc2c(-c3ccc(N(c4ccc(-c5ccc6oc7ccccc7c6c5)cc4)c4ccc(-c5ccc6sc7ccccc7c6c5)cc4)cc3)cccc2c1 ; c1ccc(-c2ccc(N(c3ccc(-c4ccc5sc6ccccc6c5c4)cc3)c3ccc(-c4cccc5ccccc45)cc3)cc2)cc1]

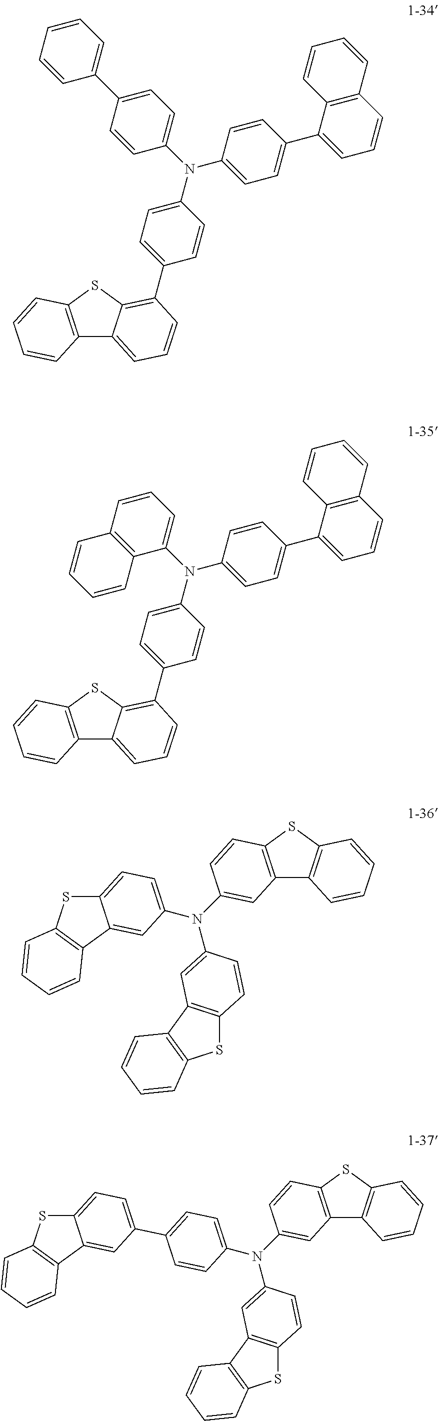 [SMILES: c1ccc(-c2ccc(N(c3ccc(-c4cccc5ccccc45)cc3)c3ccc(-c4cccc5c4sc4ccccc45)cc3)cc2)cc1 ; c1ccc2c(-c3ccc(N(c4ccc(-c5cccc6c5sc5ccccc56)cc4)c4cccc5ccccc45)cc3)cccc2c1 ; c1ccc2c(c1)sc1ccc(N(c3ccc4sc5ccccc5c4c3)c3ccc4sc5ccccc5c4c3)cc12 ; c1ccc2c(c1)sc1ccc(-c3ccc(N(c4ccc5sc6ccccc6c5c4)c4ccc5sc6ccccc6c5c4)cc3)cc12]

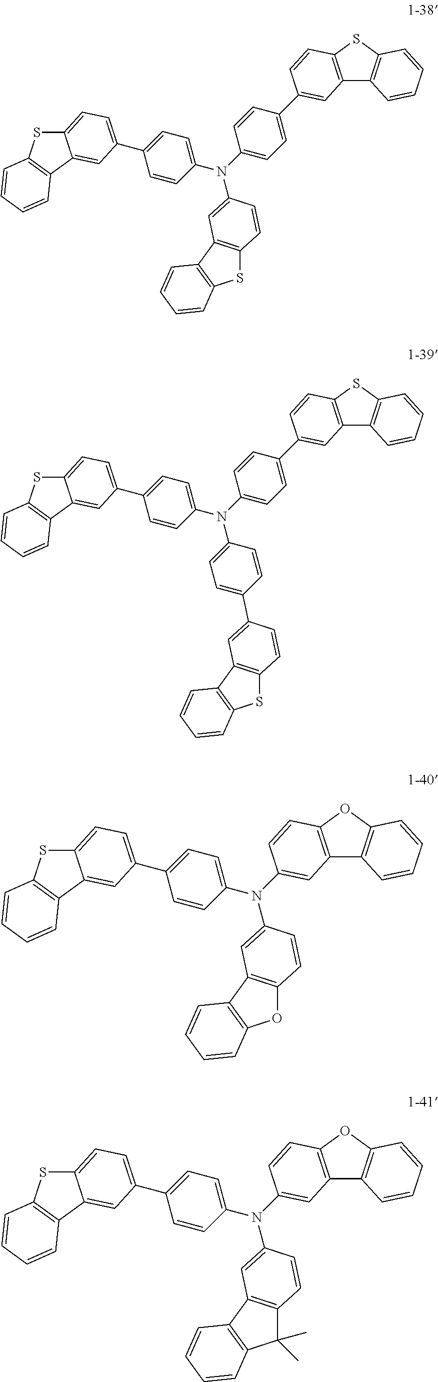 [SMILES: c1ccc2c(c1)sc1ccc(-c3ccc(N(c4ccc(-c5ccc6sc7ccccc7c6c5)cc4)c4ccc5sc6ccccc6c5c4)cc3)cc12 ; c1ccc2c(c1)sc1ccc(-c3ccc(N(c4ccc(-c5ccc6sc7ccccc7c6c5)cc4)c4ccc(-c5ccc6sc7ccccc7c6c5)cc4)cc3)cc12 ; c1ccc2c(c1)oc1ccc(N(c3ccc(-c4ccc5sc6ccccc6c5c4)cc3)c3ccc4oc5ccccc5c4c3)cc12 ; CC1(C)c2ccccc2-c2cc(N(c3ccc(-c4ccc5sc6ccccc6c5c4)cc3)c3ccc4oc5ccccc5c4c3)ccc21]

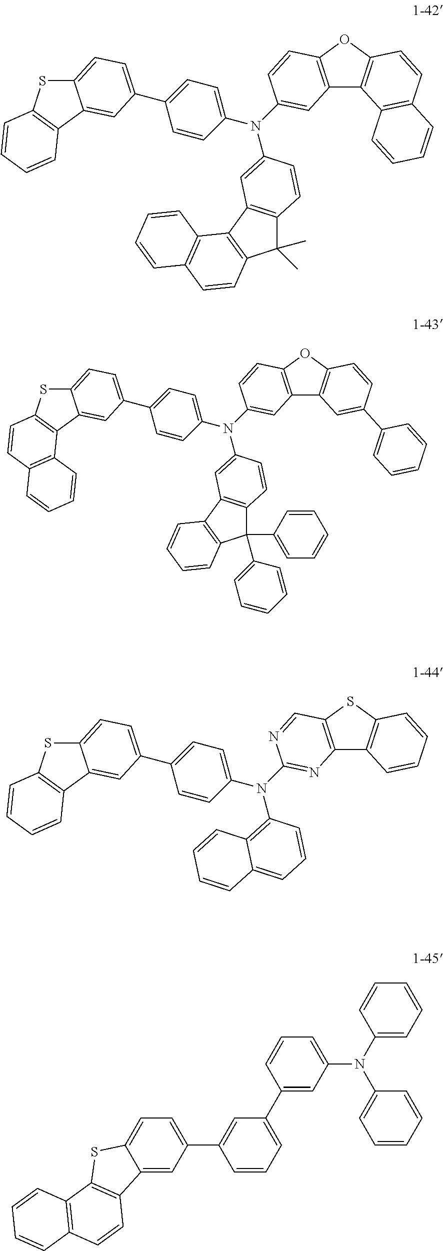 [SMILES: CC1(C)c2ccc(N(c3ccc(-c4ccc5sc6ccccc6c5c4)cc3)c3ccc4oc5ccc6ccccc6c5c4c3)cc2-c2c1ccc1ccccc21 ; c1ccc(-c2ccc3oc4ccc(N(c5ccc(-c6ccc7sc8ccc9ccccc9c8c7c6)cc5)c5ccc6c(c5)-c5ccccc5C6(c5ccccc5)c5ccccc5)cc4c3c2)cc1 ; c1ccc2c(N(c3ccc(-c4ccc5sc6ccccc6c5c4)cc3)c3ncc4sc5ccccc5c4n3)cccc2c1 ; c1ccc(N(c2ccccc2)c2cccc(-c3cccc(-c4ccc5sc6c7ccccc7ccc6c5c4)c3)c2)cc1]

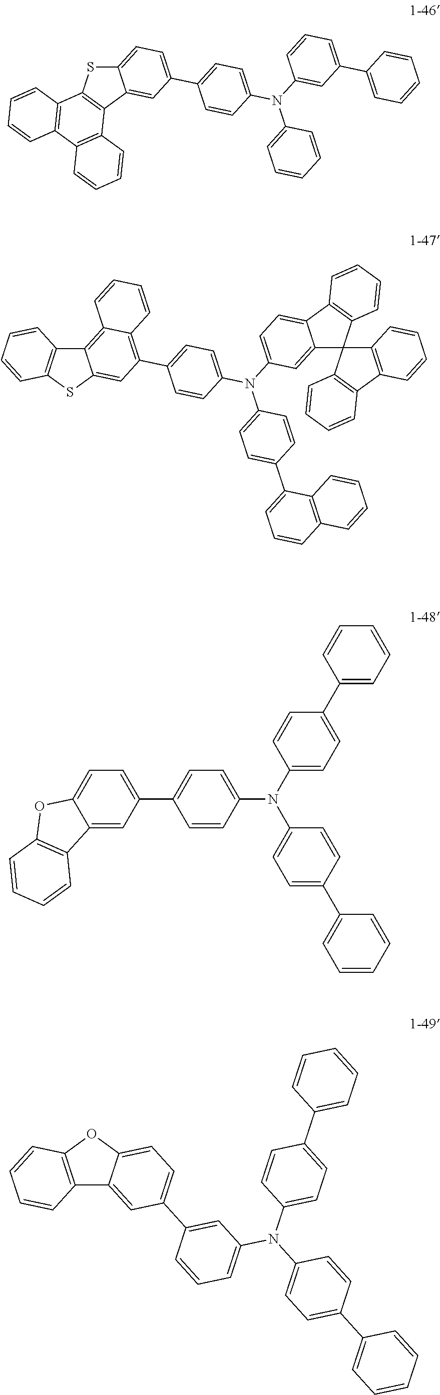 [SMILES: c1ccc(-c2cccc(N(c3ccccc3)c3ccc(-c4ccc5sc6c7ccccc7c7ccccc7c6c5c4)cc3)c2)cc1 ; c1ccc2c(c1)-c1ccccc1C21c2ccccc2-c2ccc(N(c3ccc(-c4cccc5ccccc45)cc3)c3ccc(-c4cc5sc6ccccc6c5c5ccccc45)cc3)cc21 ; c1ccc(-c2ccc(N(c3ccc(-c4ccccc4)cc3)c3ccc(-c4ccc5oc6ccccc6c5c4)cc3)cc2)cc1 ; c1ccc(-c2ccc(N(c3ccc(-c4ccccc4)cc3)c3cccc(-c4ccc5oc6ccccc6c5c4)c3)cc2)cc1]

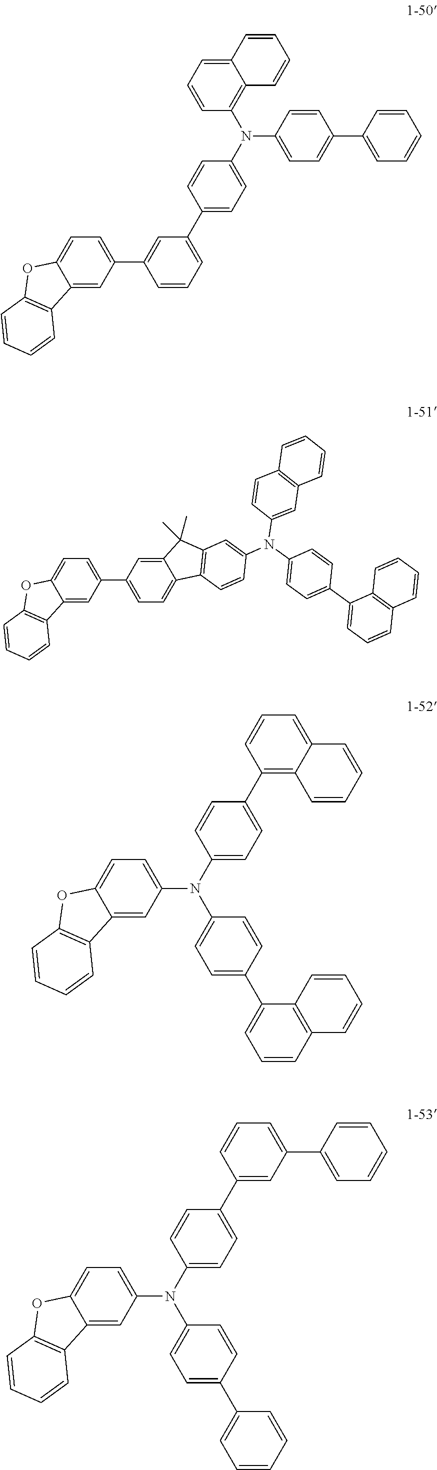 [SMILES: c1ccc(-c2ccc(N(c3ccc(-c4cccc(-c5ccc6oc7ccccc7c6c5)c4)cc3)c3cccc4ccccc34)cc2)cc1 ; CC1(C)c2cc(-c3ccc4oc5ccccc5c4c3)ccc2-c2ccc(N(c3ccc(-c4cccc5ccccc45)cc3)c3ccc4ccccc4c3)cc21 ; c1ccc2c(-c3ccc(N(c4ccc(-c5cccc6ccccc56)cc4)c4ccc5oc6ccccc6c5c4)cc3)cccc2c1 ; c1ccc(-c2ccc(N(c3ccc(-c4cccc(-c5ccccc5)c4)cc3)c3ccc4oc5ccccc5c4c3)cc2)cc1]

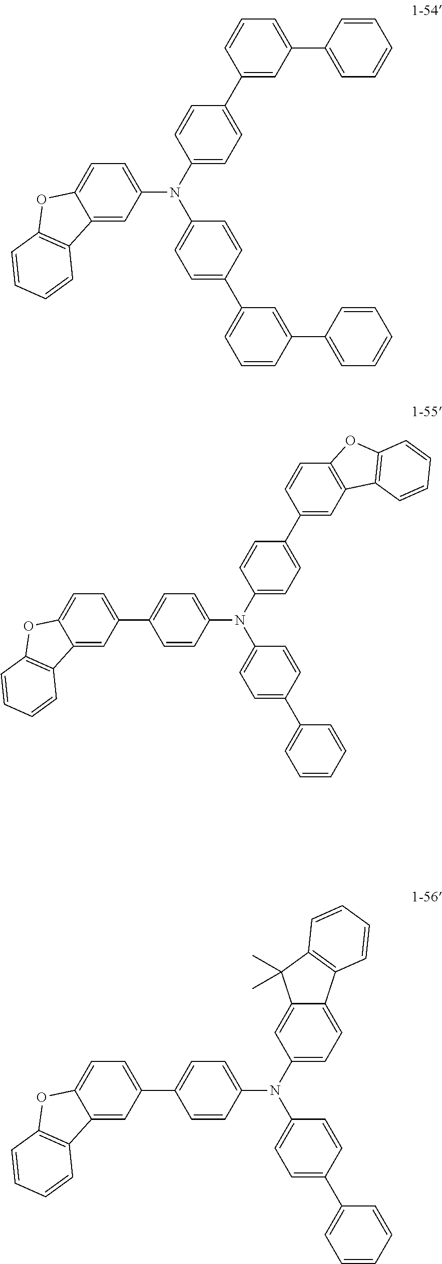 [SMILES: c1ccc(-c2cccc(-c3ccc(N(c4ccc(-c5cccc(-c6ccccc6)c5)cc4)c4ccc5oc6ccccc6c5c4)cc3)c2)cc1 ; c1ccc(-c2ccc(N(c3ccc(-c4ccc5oc6ccccc6c5c4)cc3)c3ccc(-c4ccc5oc6ccccc6c5c4)cc3)cc2)cc1 ; CC1(C)c2ccccc2-c2ccc(N(c3ccc(-c4ccccc4)cc3)c3ccc(-c4ccc5oc6ccccc6c5c4)cc3)cc21]

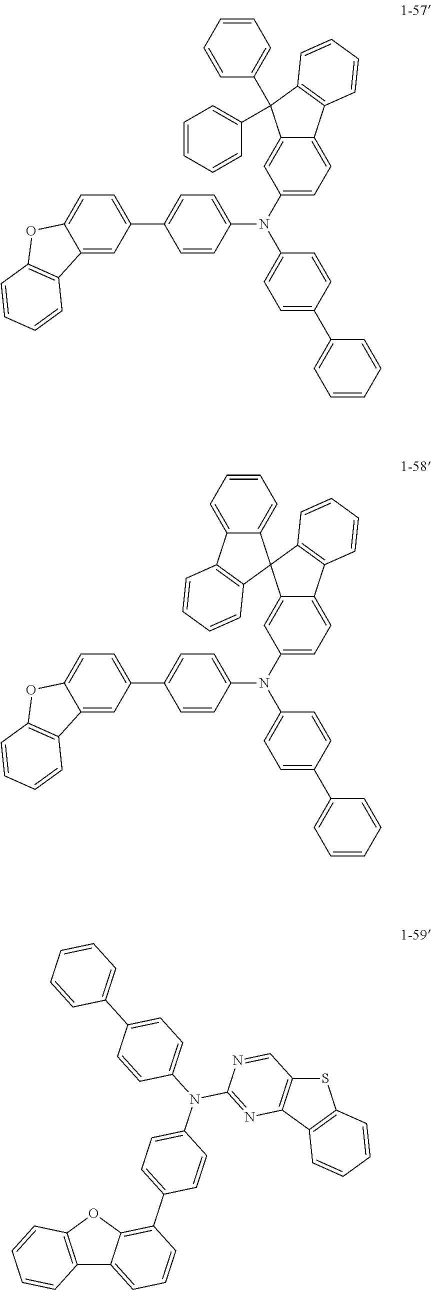 [SMILES: c1ccc(-c2ccc(N(c3ccc(-c4ccc5oc6ccccc6c5c4)cc3)c3ccc4c(c3)C(c3ccccc3)(c3ccccc3)c3ccccc3-4)cc2)cc1 ; c1ccc(-c2ccc(N(c3ccc(-c4ccc5oc6ccccc6c5c4)cc3)c3ccc4c(c3)C3(c5ccccc5-c5ccccc53)c3ccccc3-4)cc2)cc1 ; c1ccc(-c2ccc(N(c3ccc(-c4cccc5c4oc4ccccc45)cc3)c3ncc4sc5ccccc5c4n3)cc2)cc1]

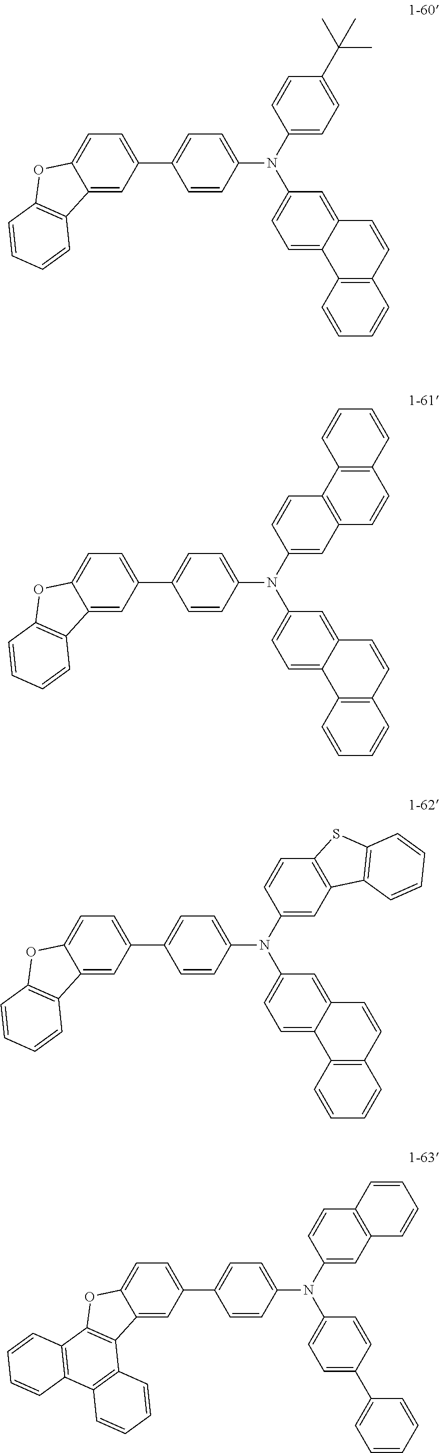 [SMILES: CC(C)(C)c1ccc(N(c2ccc(-c3ccc4oc5ccccc5c4c3)cc2)c2ccc3c(ccc4ccccc43)c2)cc1 ; c1ccc2c(c1)ccc1cc(N(c3ccc(-c4ccc5oc6ccccc6c5c4)cc3)c3ccc4c(ccc5ccccc54)c3)ccc12 ; c1ccc2c(c1)ccc1cc(N(c3ccc(-c4ccc5oc6ccccc6c5c4)cc3)c3ccc4sc5ccccc5c4c3)ccc12 ; c1ccc(-c2ccc(N(c3ccc(-c4ccc5oc6c7ccccc7c7ccccc7c6c5c4)cc3)c3ccc4ccccc4c3)cc2)cc1]

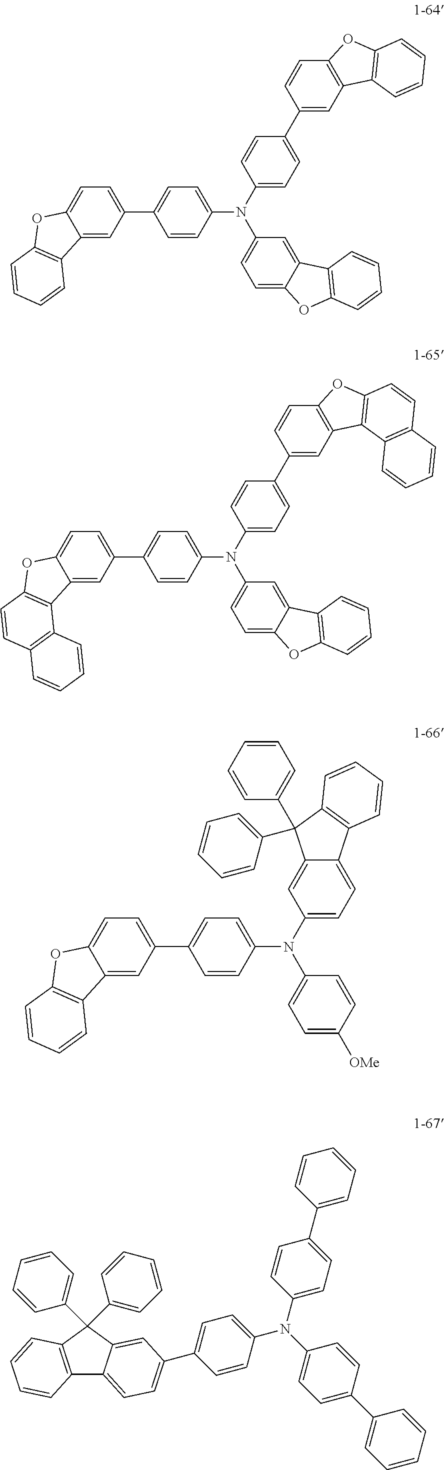 [SMILES: c1ccc2c(c1)oc1ccc(-c3ccc(N(c4ccc(-c5ccc6oc7ccccc7c6c5)cc4)c4ccc5oc6ccccc6c5c4)cc3)cc12 ; c1ccc2c(c1)ccc1oc3ccc(-c4ccc(N(c5ccc(-c6ccc7oc8ccc9ccccc9c8c7c6)cc5)c5ccc6oc7ccccc7c6c5)cc4)cc3c12 ; COc1ccc(N(c2ccc(-c3ccc4oc5ccccc5c4c3)cc2)c2ccc3c(c2)C(c2ccccc2)(c2ccccc2)c2ccccc2-3)cc1 ; c1ccc(-c2ccc(N(c3ccc(-c4ccccc4)cc3)c3ccc(-c4ccc5c(c4)C(c4ccccc4)(c4ccccc4)c4ccccc4-5)cc3)cc2)cc1]

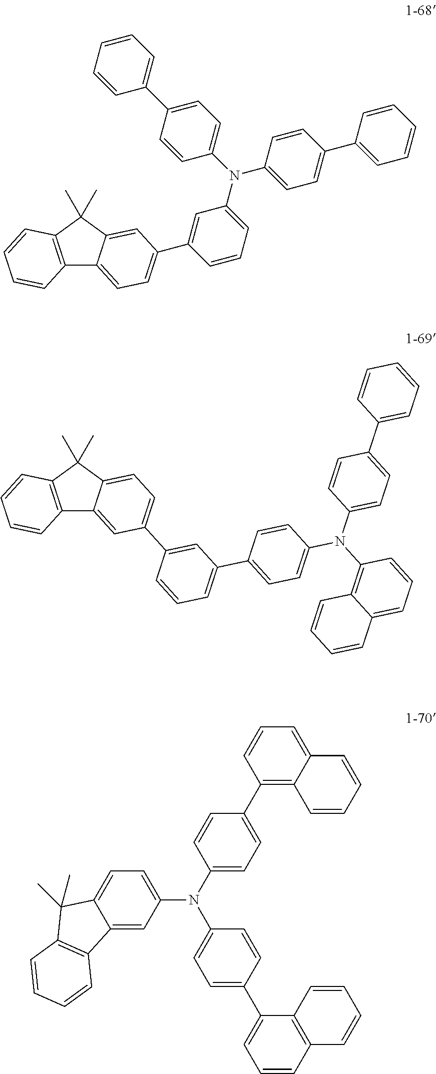 [SMILES: CC1(C)c2ccccc2-c2ccc(-c3cccc(N(c4ccc(-c5ccccc5)cc4)c4ccc(-c5ccccc5)cc4)c3)cc21 ; CC1(C)c2ccccc2-c2cc(-c3cccc(-c4ccc(N(c5ccc(-c6ccccc6)cc5)c5cccc6ccccc56)cc4)c3)ccc21 ; CC1(C)c2ccccc2-c2cc(N(c3ccc(-c4cccc5ccccc45)cc3)c3ccc(-c4cccc5ccccc45)cc3)ccc21]

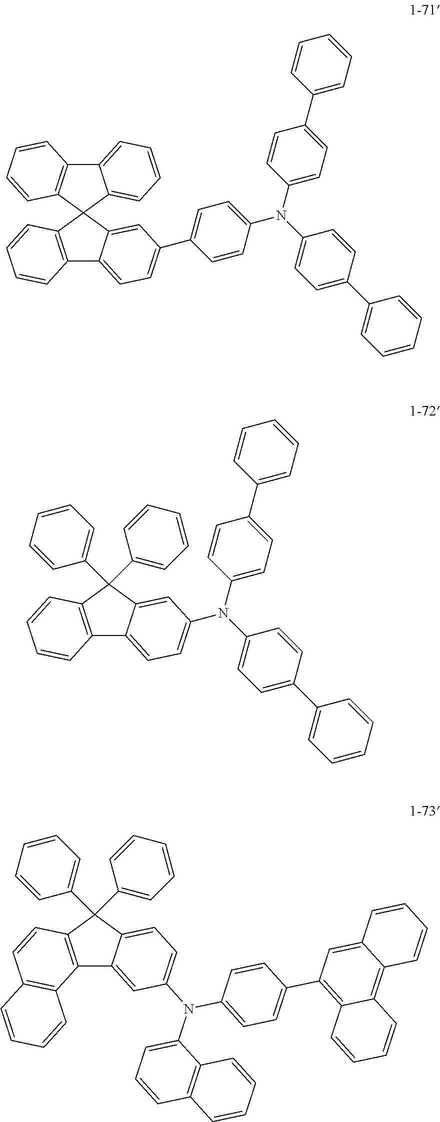 [SMILES: c1ccc(-c2ccc(N(c3ccc(-c4ccccc4)cc3)c3ccc(-c4ccc5c(c4)C4(c6ccccc6-c6ccccc64)c4ccccc4-5)cc3)cc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1 ; c1ccc(C2(c3ccccc3)c3ccc(N(c4ccc(-c5cc6ccccc6c6ccccc56)cc4)c4cccc5ccccc45)cc3-c3c2ccc2ccccc32)cc1]

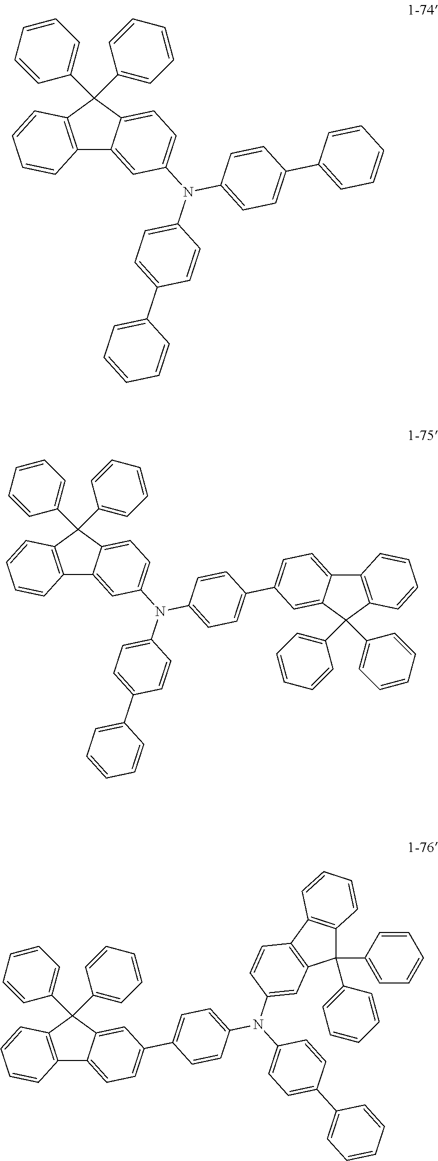 [SMILES: c1ccc(-c2ccc(N(c3ccc(-c4ccccc4)cc3)c3ccc4c(c3)-c3ccccc3C4(c3ccccc3)c3ccccc3)cc2)cc1 ; c1ccc(-c2ccc(N(c3ccc(-c4ccc5c(c4)C(c4ccccc4)(c4ccccc4)c4ccccc4-5)cc3)c3ccc4c(c3)-c3ccccc3C4(c3ccccc3)c3ccccc3)cc2)cc1 ; c1ccc(-c2ccc(N(c3ccc(-c4ccc5c(c4)C(c4ccccc4)(c4ccccc4)c4ccccc4-5)cc3)c3ccc4c(c3)C(c3ccccc3)(c3ccccc3)c3ccccc3-4)cc2)cc1]

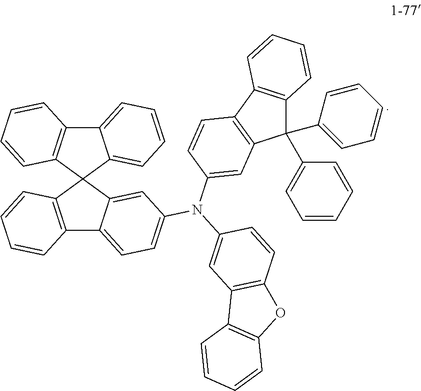 [SMILES: c1ccc(C2(c3ccccc3)c3ccccc3-c3ccc(N(c4ccc5c(c4)C4(c6ccccc6-c6ccccc64)c4ccccc4-5)c4ccc5oc6ccccc6c5c4)cc32)cc1]

3. The organic electric element according to claim 1, wherein either of the two compounds having different structures has a mixing ratio of 10% to 90%.

4. The organic electric element according to claim 1, wherein the two compounds of the mixture having different structures has a mixing ratio of 5:5, 6:4, 7:3, 8:2, and 9:1.

5. The organic electric element according to claim 1, wherein the mixture of the two compounds having different structures further comprises one or more compounds of Formula 1-2, 1-3 or 1-4.

6. The organic electric element according to claim 1 further comprising an emitting auxiliary layer between the emitting layer and the hole transport layer composed of the mixture of the two compounds having different structures, and the emitting auxiliary layer comprises a compound represented by Formula 1-2, 1-3 or 1-4.

7. The organic electric element according to claim 1, further comprising a light efficiency improving layer formed on at least one side of the sides of the first electrode and the second electrode, the one side being opposite to the organic material layer.

8. The organic electric element according to claim 1, wherein the organic material layer is formed by one of a spin coating process, a nozzle printing process, an inkjet printing process, a slot coating process, a dip coating process, and a roll-to-roll process.

9. An electronic device comprising a display device comprising the organic electric element of claim 1; and a control part driving the display device.

10. The electronic device according to claim 9, wherein the organic electric element is an OLED, an organic solar cell, an organic photo conductor (OPC), Organic transistor (organic TFT), or an element for monochromic or white illumination.
